(19) United States
(12) Patent Application Publication
(10) Pub. No.: US 2019/0225978 A1
(43) Pub. Date: Jul. 25, 2019

(54) MODIFYING N-GLYCOSYLATION OF PLANT PROTEINS USING GDP-4-DEHYDRO-6-DEOXY-D-MANNOSE REDUCTASE (RMD)

(71) Applicants: MEDICAGO INC., QUEBEC (CA); GENOPOLE, EVRY (FR)

(72) Inventors: MARC-ANDRE D'AOUST, QUEBEC (CA); PIERRE-OLIVIER LAVOIE, QUEBEC (CA); ANDEOL FALCON DE LONGEVIALLE, EVRY CEDEX (FR)

(21) Appl. No.: 16/338,163

(22) PCT Filed: Sep. 29, 2017
(86) PCT No.: PCT/CA2017/051165

§ 371 Date: Mar. 29, 2019

Related U.S. Application Data
(60) Provisional application No. 62/402,022, filed on Sep. 30, 2016.

(51) Int. Cl. C12N 15/82; C07K 16/28; C12N 9/04

(52) U.S. Cl. CPC C12N 15/8257; C07K 16/2887; C07K 2317/13; C12Y 101/01281; C12N 15/8218; C12N 9/0006

(57) ABSTRACT

A method for synthesizing a protein of interest with a modified N-glycosylation profile within a plant, a portion of a plant, or a plant cell is provided. The method comprises co-expressing within a plant a nucleotide sequence encoding a first nucleotide sequence encoding a GDP-4-dehydro-6-deoxy-D-mannose reductase (RMD) the first nucleotide sequence operatively linked with a first regulatory region that is active in the plant, and a second nucleotide sequence encoding the protein of interest, the second nucleotide sequence operatively linked with a second regulatory region that is active in the plant. The first and second nucleotide sequences are co-expressed to synthesize a protein of interest comprising glycans with the modified N-glycosylation profile within the plant, the portion of the plant, or the plant cell.

### FIELD OF INVENTION

The present invention relates to methods for modifying glycoprotein production in plants using GDP-4-dehydro-6-deoxy-D-mannose reductase (RMD). The present invention also provides plants with modified glycoprotein production.

### BACKGROUND OF THE INVENTION

Plants are an attractive alternative for the production of recombinant proteins, however, their inability to perform authentic mammalian N-glycosylation may result in limitations for their use in the production of therapeutics. A possible concern is the presence of beta1,2-xylose and core alpha1,3-fucose residues on complex N-linked glycans, as these N-glycan epitopes may be immunogenic in mammals. For example the presence of core alpha (1,3)-fucose on the N-glycan of the Fc region of monoclonal antibodies is known to significantly reduce antibody-dependent cell-mediated cytotoxicity (ADCC) activity of the antibody (Cox K. M. et. al., 2006, Nat. Biotech 24:1591-1597).

N-glycan maturation takes place within the ER and Golgi, and involves trimming of sugar residues from an oligosaccharide precursor of N-glycans using localized glycosidases to produce a Man5GlcNAc2 structure. Further processing involves transfer of sugar residues from nucleotide sugar donors onto the N-glycans via Golgi-localized glycosyltransferases. In mammalian cells, and plant cells, the glycosidases and glycosyltransferases are distributed along the Golgi from the cis- to the trans-regions in the order in which they process N-glycans. The N-linked glycosylation mechanisms in mammalian and plant systems have been conserved during evolution. However, differences are observed in the final steps of oligosaccharide trimming and glycan modification in the Golgi apparatus. The later steps of N-glycosylation in mammalian cells add β1,4galactose, α1,6fucose (beta-1,4galactose, alpha-1,6fucose) and terminal sialic acid residues to complex glycans. However, in plants β1,3galactose, α1,3fucose (beta-1,3galactose, alpha-1,3fucose), α1,4fucose and β1,2xylose (alpha-1,4fucose and beta-1,2xylose) residues are added (see Vezina—et. al., 2009, Plant Biotech. J., pp 442-455; Saint-Jore-Dupas C., et. al., 2006, Plant Cell 18:3182-3200; for overview). As a result, higher plants mainly generate complex-type glycans with an α-1,3 fucose residue attached to the innermost GlcNAc and a β-1,2 xylose residue attached to the junction mannose of the tri-mannosyl core, neither of which is found in humans. Thus biopharmaceutical glycoproteins produced in plants carry N-glycans with plant-specific residues core α(1,3)-fucose and β(1,2)-xylose, which can significantly impact the activity, stability and immunogenicity of biopharmaceuticals.

In order to modify the sugar chain structure of the produced glycoprotein, various methods have been attempted, such as 1) application of an inhibitor against an enzyme relating to the modification of a sugar chain, 2) homozygous knock out of a gene involved in sugar synthesis or transfer 3) selection of a mutant, 4) introduction of a gene encoding an enzyme relating to the modification of a sugar chain, and the like.

One approach to alter fucosylation in mammalian cell lines has been to knockout intrinsic α-1,6-fucosyltransferase (FUT8) enzyme activity, which is responsible for core fucosylation. Other recombinant DNA-based glycoengineering approaches have been achieved through overexpression of heterologous β-1,4-N-acetylglucosaminyltransferase III (GnT-III). GnT-III adds a bisecting GlcNAc to an oligosaccharide which sterically blocks core-fucosylation and overexpression of heterologous GDP-6-deoxy-D-lyxo-4-hexulose reductase also referred to as GDP-4-keto-6-deoxy-D)-mannose reductase, abbreviated (RMD; von Horsten et al 2010, Glycobiology vol. 20 no. 12 pp. 1607-1618).

Cells have fucosyltransferases that add a fucose residue to the GlcNAc residue at the reducing end of the N-glycans on a protein or to other nascent glycostructures on glycolipids. Fucosylation of protein- or lipid-bound glycomoieties requires a nucleotide sugar, GDP-L-fucose, as a donor and also the presence of particular fucosyl transferases, which transfer the fucosyl residue from the donor to the acceptor molecule. In vertebrate cells and plants, GDP-L-fucose can be synthesized via two different pathways, either by the more prominent fucose de novo pathway or by the minor salvage pathway. It is believed that insect cells do not possess the salvage pathway.

The more prominent fucose de novo pathway starts from GDP-D-mannose and consists of a GDP-mannose dehydratase (GMD) and GDP-keto-deoxy-mannose-epimerase/GDP-keto-deoxy-galactose-reductase (GMER, also known as Fx in humans), both located in the cytoplasm, which in concert converts GDP-mannose to GDP-L-fucose (FIG. 1). GMD is conserved throughout evolution in bacterial species, plants, invertebrates, and mammals.

In the first reaction step, GMD converts GDP-mannose to GDP-4-keto-6-deoxymannose by catalyzing the oxidation of the hydroxyl group at C-4 of the mannose ring coupled with reduction of the hydroxyl at C-6 (FIG. 1).

GDP-4-keto-6-deoxymannose produced by GMD is then converted to GDP-fucose by the dual functional epimerase-reductase enzyme GMER. In the first reaction carried out by GMER, the hydroxyl group at C-3 and the methyl group at C-5 of the mannose ring are epimerized to yield GDP-4-keto-6-deoxygalactose.

The 4-reductase activity GMER then catalyzes a hydride transfer from the required Nicotinamide adenine dinucleotide phosphate, reduced form, (NADPH) cofactor to the keto group at C-4, yielding GDP-fucose and NADP+ (FIG. 1).

Later, GDP-L-fucose is transported into the Golgi via a GDP-fucose transporter located in the membrane of the Golgi apparatus. Once GDP-L-fucose has entered the Golgi luminal compartment, fucosyltransferases can covalently link GDP-L-fucose to nascent glycomoieties within the Golgi.

Similar to vertebrates and bacteria, the biosynthesis of L-Fucose in plants occurs through the conversion of GDP-d-mannose to GDP-L-Fucose in three catalytic steps: 4,6-dehydration, 3,5-epimerization, and 4-reduction. These activities are carried out by two enzymes, a GDP-d-mannose 4,6-dehydratase, and a GDP-4-keto-6-deoxy-d-Mannose (GDP-KDM) 3,5-epimerase-4-reductase (synonymous with GDP-1-Fucose synthase, FX protein).

In mammals and plants, an alternative salvage pathway or “scavenger” pathway can yield GDP-fucose derived directly from fucose. The salvage pathway is a minor source of GDP-L-fucose (circa 10%) which can be blocked by omission of free fucose and fucosylated glycoproteins from the culture medium. The salvage pathway starts from extracellular fucose which can be transported into the cytosolic compartment via fucose-specific plasma membrane transporters. Alternatively, fucose cleaved from endocytosed glycoproteins can enter the cytosol. In the salvage pathway, cytosolic L-fucose is phosphorylated by fucokinase to fucose-1-phosphate. GDP-fucose pyrophosphorylase (GFPP) then catalyzes the reversible condensation of fucose-1-phosphate with GTP to form GDP-fucose (FIG. 1).

Von Horsten et al. (Glycobiology vol. 20 no. 12 pp. 1607-1618, 2010) produced non-fucosylated antibodies by co-expressing the antibody along with a heterologous bacterial GDP-6-deoxy-D-lyxo-4-hexulose reductase (RMD) in mammalian cells. Antibody-producing Chinese hamster ovary (CHO) cells that were modified in this way secreted antibodies lacking core fucose. Similarly, U.S. Pat. No. 8,642,292 discloses vertebrate cells expressing heterologous RMD. These cells produce antibodies that lack fucose or have a reduced amount of fucose on their glycomoieties.

U.S. Pat. No. 8,642,292 described co-expression of an IgG with RMD in CHO cells. The nucleotide sequence encoding RMD was expressed under the control of a constitutive promoter and in the absence of an expression enhancer. The expressed IgG was observed to have a 98% reduction in fucosylation.

US 2014/0221627 discloses a method for producing molecules having atypical fucose analogues on their glycomoieties or amino acids. The GDP-L-fucose synthesis pathway originating from GDP-D-mannose (de novo pathway) is blocked in mammalian cells by expressing RMD, along with adding a GDP-L-fucose analogue for integration into their glycomoieties or amino acids, to the cell. The fucose analogues may be used to specifically couple pharmaceutically active compounds to molecules such as proteins or lipids, to which they are attached.

Mabashi-Asazuma et al. (Glycobiology vol. 24 no. 3 pp. 325-340, 2014) developed a new baculovirus-insect cell system that can produce nonfucosylated re-combinant glycoproteins. Insect cell lines were prepared that constitutively expressed a Pseudomonas aeruginosa gene encoding GDP-4-dehydro-6-deoxy-D-mannose reductase (RMD), which consumed the immediate precursor to GDP-L-fucose, and blocked core α1,6-fucosylation (in a manner similar to that taught in von Horsten et al. 2010 discussed above, in Chinese hamster ovary (CHO) cells). Mabashi-Asazuma et al. found that while this approach appeared to be temporarily effective, they observed that it could not be used successfully in the baculovirus-insect cell system because the fucosylation-negative phenotype induced by constitutive RMD expression in insect cell lines was unstable. This result revealed that the approach to block core α1,6-fucosylation in CHO cells could not be used in insect cell systems. Thus, Mabashi-Asazuma et al. focused on glycoengineering using the baculovirus vector, rather than the host. Mabashi-Asazuma et al. constructed a novel baculovirus vector designed to express RMD immediately after infection with the gene of interest (under the control of an immediate-Early (IE) promoter, Pie1), and to facilitate downstream isolation of daughter vectors capable of expressing recombinant glycoproteins of interest later in infection. Using this method they isolated a daughter vector encoding a nonfucosylated recombinant therapeutic anti-CD20-immunoglobulin G (IgG), rituxi mab.

WO 2015/057393 describes blocking of biosynthesis of GDP-L-fucose in insect cell lines. WO 2015/057393 states that insects appeared to be the only multicellular organisms lacking two enzymes, L-fucokinase (FUK) and L-fucose-1-phosphate guanylyltransferase (FPGT), required for the GDP-L-fucose salvage pathway, thus making this approach particular attractive for insect cells. WO 2015/057393 expressed Pseudomonas aeruginosa RMD in insect cells together with Fc domain of mouse IgG2a (mIgG2a-Fc), and found that the fucosylation-negative phenotype is unstable in insect cell lines. In view of this phenotypic instability of insect cells, they abandoned their efforts to glycolengineer the host cell component and focused their attention on the baculoviral vector component of the baculovirus-insect cell system.

Palmberger et al. (Biotechnol J. 2014 September; 9(9): 1206-1214.) evaluated the impact of fucose residues on the allergenic potential of an insect cell-expressed vaccine candidate. In order to block the GDP-L-fucose de novo synthesis pathway Pseudomonas aeruginosa GDP-6-deoxy-D-lyxo-4-hexulose reductase (RMD) gene was integrated into a baculovirus backbone. This virus was then used for the expression of soluble influenza A virus hemagglutinin. The co-expression of RMD in insect cell lines leads to a shift of the dominant structures towards nonfucosylated tri-mannose structures.

### SUMMARY OF THE INVENTION

The present invention relates to methods for modifying glycoprotein production in plants using GDP-4-dehydro-6-deoxy-D-mannose reductase (RMD). The present invention also provides plants with modified glycoprotein production.

It is an object of the invention to provide an improved method for modifying glycoprotein production in plants.

There is provided herein a method (A) of producing a protein of interest comprising N-glycans with a modified N-glycosylation profile in a plant comprising, co-expressing within a plant, a portion of a plant, or a plant cell, a first nucleotide sequence encoding a GDP-4-dehydro-6-deoxy-D-mannose reductase (RMD) the first nucleotide sequence operatively linked with a first regulatory region that is active in the plant, the portion of the plant, or the plant cell, and a second nucleotide sequence for encoding the protein of interest, the second nucleotide sequence operatively linked with a second regulatory region that is active in the plant, the portion of the plant, or the plant cell, and co-expressing the first and second nucleotide sequences to synthesize a protein of interest comprising glycans with the modified N-glycosylation profile when compared to the N-glycosylation profile of the protein of interest expressed in a second (control) plant, a portion of a second plant, or a second plant cell that does not express RMD.

The regulatory region of the first nucleotide sequence, the second nucleotide sequence, or both the first and second nucleotide sequence, described in method (A) above, may comprise an expression enhancer. The expression enhancer may be selected of CPMVX, CPMVX+, CPMV-HT+CPMV HT+[WT 115] or CPMV HT+[511]. The RMD may be derived from Pseudomonas, Xanthomonas, Agrobacterium, a bacterial source, or other source. For example the RMD may be selected from paRMD, atRMD, pbRMD, psRMD, or xvRMD.

The plant, portion of the plant, or plant cell described in method (A) above may further exhibit reduced, or lack, β(1,2)-xylosyltransferase (XylT) activity, α(1,3)-fucosyltransferase (FucT) activity, or both β(1,2)-xylosyltransferase (XylT) and α(1,3)-fucosyltransferase (FucT) activities. For example, the FucT, XylT, or both the FucT and XylT genes in the plant, portion of the plant, or plant cell may be knocked out, or the FucT activity may be reduced using RNAi, chemical inhibition, or both.

A method (B) of producing a protein of interest comprising N-glycans with a reduced fucose content in a plant, a portion of a plant, or a plant cell having reduced fucosylation activity is also provided. The method (B) comprises, co-expressing within the plant, the portion of the plant, or the plant cell having reduced fucosylation activity, a nucleotide sequence encoding a first nucleotide sequence encoding a GDP-4-dehydro-6-deoxy-D-mannose reductase (RMD) the first nucleotide sequence operatively linked with a first regulatory region that is active in the plant, the portion of a plant, or the plant cell, and a second nucleotide sequence encoding the protein of interest, the second nucleotide sequence operatively linked with a second regulatory region that is active in the plant, the portion of a plant, or the plant cell, and co-expressing the first and second nucleotide sequences to synthesize a protein of interest comprising N-glycans with a reduced fucose content, when compared to the fucose content of the protein of interest expressed in the plant, the portion of the plant, or the plant cell that does not express RMD.

The regulatory region of the first nucleotide sequence, the second nucleotide sequence, or both the first and second nucleotide sequence, described in method (B) above, may comprise an expression enhancer. The expression enhancer may be selected of CPMVX, CPMVX+, CPMV-HT+CPMV HT+[WT 115] or CPMV HT+[511]. The RMD may be derived from Pseudomonas, Xanthomonas, Agrobacterium, a bacterial source, or other source. For example the RMD may be selected from paRMD, atRMD, pbRMD, psRMD, or xvRMD.

The plant, portion of the plant, or plant cell having reduced fucosylation activity as described in method (B) above may exhibit reduced, or lack, α(1,3)-fucosyltransferase (FucT) activity, or both α(1,3)-fucosyltransferase (FucT) and β(1,2)-xylosyltransferase (XylT) activities. For example, at least one the FucT, XylT, or at least one of both of the FucT and XylT genes in the plant, portion of the plant, or plant cell may be knocked out. Alternatively, the FucT activity may be reduced using RNAi, chemical inhibition, or both.

Also provided is a method (C) of producing a protein of interest comprising N-glycans having a reduced fucose content in a plant, a portion of a plant, or a plant cell having reduced fucosylation activity and exhibiting reduced, or lacking, α(1,3)-fucosyltransferase (FucT) activity, the method comprising, co-expressing within the plant, the portion of the plant, or the plant cell, a first nucleotide sequence encoding a GDP-4-dehydro-6-deoxy-D-mannose reductase (RMD) the first nucleotide sequence operatively linked with a first regulatory region that is active in the plant, the portion of a plant, or the plant cell, and a second nucleotide sequence encoding the protein of interest, the second nucleotide sequence operatively linked with a second regulatory region that is active in the plant, the portion of a plant, or the plant cell, and co-expressing the first and second nucleotide sequences to synthesize a protein of interest comprising N-glycans having a reduced fucose content, when compared to the fucose content of the protein of interest expressed in a second plant, a portion of the second plant, or a second plant cell that does not express RMD.

The protein of interest produced by the methods (A), (B) or (C) as described above, may lack oligosaccharides residues Gn2M3XGn2, Gn2M3FGn2, Gn2M3XFGn2 or a combination thereof.

A protein of interest produced by the methods (A), (B) or (C) as described above is also provided. The protein of interest may be a therapeutic protein, an antibody, a vaccine component or a viral protein. The protein of interest may lack oligosaccharides residues Gn2M3XGn2, Gn2M3FGn2, Gn2M3XFGn2 or a combination thereof.

Also described herein is a plant, portion of a plant, or a plant cell comprising a nucleotide sequence encoding a GDP-4-dehydro-6-deoxy-D-mannose reductase (RMD). The nucleotide sequence is operatively linked with a regulatory region that is active in the plant. The plant, portion of the plant, or plant cell may further comprise a second nucleotide sequence for encoding a protein of interest, the second nucleotide sequence operatively linked with a second regulatory region that is active in the plant. The plant, portion of the plant, or plant cell as described above may comprise reduced level of GDP-L-fucose when compared to a plant, portion of a plant, or a plant cell that does not comprise RMD. The plant, portion of the plant, or plant cell may exhibit reduced, or lack, α(1,3)-fucosyltransferase (FucT) activity, or both α(1,3)-fucosyltransferase (FucT) and β(1,2)-xylosyltransferase (XylT) activities. For example, at least one the FucT, XylT, or at least one of both of the FucT and XylT genes in the plant, portion of the plant, or plant cell may be knocked out. Alternatively, the FucT activity may be reduced using RNAi, chemical inhibition, or both.

Also provided herein is a method for producing a protein of interest in a plant of the Nicotiana spp having at least one of its FucT allele knocked-out comprising co-expressing a GDP-4-dehydro-6-deoxy-D-mannose reductase (RMD) and the protein of interest within the plant, to produce the protein of interest having a reduced fucosylation profile when compared to the same protein of interest produced in a wild-type plant.

Without wishing to be bound by theory, by expressing RMD within a plant, a portion of a plant, or a plant cell, the pool of available fucose accessed by the N-glycosylation machinery is reduced, which results in reducing the fucose content of co-expressed protein of interest. Furthermore, by expressing RMD within a plant, a portion of a plant, or a plant cell having reduced fucosylation activity, the pool of available fucose accessed by the N-glycosylation machinery is reduced, thereby producing a co-expressed protein of interest having an N-glycosylation profile with reduced fucose content.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows an overview of the de novo and fucose salvage pathways in eukaryotic cells. In the absence of fucose, cells are unable to synthesize GDP-fucose via the salvage pathway (see right hand panel). The de novo pathway can be blocked by enzymatic conversion of the intermediate GDP-4-keto-6-deoxymannose by GDP-6-deoxy-D-lyxo-4-hexylose reductase (RMD) into the dead end product GDP-D-rhamnose that typically does not occur in eukaryotic cells (left hand panel). GDP-D-rhamnose may exert a feedback inhibition on the GMD-enzyme thereby further blocking the fucose de novo pathway as well as the alternate GDP-rhamnose synthesis. The salvage pathway may be blocked by avoidance of an external fucose source, or by converting L-Fucose into L-Fucono-1-5 lactone (via L Fucose dehydrogenase), which is then further converted into L-Fucono-1-4-lactone.

FIG. 2 shows protein staining of SDS-PAGE analysis of crude extract from plants expressing Plasto/Flag-RMD (Flag-RMD under the control of plastocyanin promoter; construct number 1191), 160+/Flag-RMD (Flag-RMD under the control of CPMV 160+; construct number 5091) or 160/Flag-RMD (Flag RMD under the control of CPMV 160; construct number 5092). The OD (optical density) of each bacterial vector used at infiltration (see methods) is indicated in parenthesis. Plants were incubated for 6 DPI; 2 μg of total soluble protein of crude plant extract per lane. The estimated molecular weight of the Flag-RMD is 35 Kda (arrow).

FIG. 3 shows protein staining of SDS-PAGE analysis of crude extract from plants expressing Ritux (rituximab, under the control of CPMV 160+; construct number 5072), or co-expressing rituximab and RMD. Ritux+160+/Flag-RMD: rituximab, under the control of CPMV 160+; construct number 5072, co-expressed with Flag-RMD, under the control of CPMV 160+; construct number 5091; or Ritux+160/Flag-RMD: rituximab, under the control of CPMV 160+; construct number 5072, co-expressed with Flag-RMD, under the control of CPMV 160; construct number 5092). OD (optical density) of each bacterial vector used at infiltration (see methods) is indicated between parentheses. Plants were incubated for 7 DPI; 2 μg of total soluble protein of crude plant extract per lane.

FIG. 4 shows SDS-PAGE and western blot analysis, probed with anti-al-3Fucose (upper panel) or anti-IgG1 (lower panel), of crude extract from plants expressing rituximab alone, or co-expressing rituximab and RMD. Ritux: rituximab under the control of CPMV 160+; construct number 5072; Ritux+160+/Flag-RMD: rituximab under the control of CPMV 160+; construct number 5072, co-expressed with Flag-RMD under the control of CPMV 160+; construct number 5091; Ritux+160/Flag-RMD: rituximab under the control of CPMV 160+; construct number 5072, co-expressed with Flag-RMD under the control of CPMV 160; construct number 5092. OD of each bacterial vector used at infiltration is indicated between parentheses. Plants were incubated for 7 DPI; 0.5 μg of total soluble protein of crude plant extract per lane. Anti-Fucose serum (1:10 000) was used to probe for fucose residues. Anti-IgG1 human Jackson Immunoresearch serum (1:7500) was used to probe for rituximab expression.

FIG. 5 shows protein staining of SDS-PAGE analysis of crude extract from plants expressing RMD, rituximab, or co-expressing RMD and rituximab. 160+/RMD: RMD under the control of CPMV 160+; construct number 5093; 160/RMD: RMD under the control of CPMV 160; construct number 5094; Ritux: rituximab under the control of CPMV 160+; construct number 5072; Ritux+160+/RMD: rituximab under the control of CPMV 160+; construct number 5072, co-expressed with RMD under the control of CPMV 160+; construct number 5093; Ritux+160/RMD: rituximab under the control of CPMV 160+; construct number 5072, co-expressed with RMD under the control of CPMV 160; construct number 5094. OD of each bacterial vector used at infiltration is indicated between parentheses. Plants were incubated for 7 DPI; 2 μg of total soluble protein of crude plant extract per lane.

FIG. 6 shows SDS-PAGE and western blot analysis, probed with anti-al-3Fucose (upper panel) or anti-IgG1 (lower panel), of crude extract from plants expressing rituximab alone, or co-expressing rituximab and RMD. Ritux: rituximab under the control of CPMV 160+; construct number 5072; Ritux+160+/RMD: rituximab under the control of CPMV 160+; construct number 5072, co-expressed with RMD under the control of CPMV 160+; construct number 5093; Ritux+160/RMD: rituximab under the control of CPMV 160+; construct number 5072, co-expressed with RMD under the control of CPMV 160; construct number 5094. OD of each bacterial vector used at infiltration is indicated between parentheses. Plants were incubated for 7 DPI; 0.5 μg of total soluble protein of crude plant extract per lane. Anti-Fucose serum (1:10 000) was used to probe for fucose residues. Anti-IgG1 human Jackson Immunoresearch serum (1:7500) was used to probe for rituximab expression.

FIG. 7 shows SDS-PAGE and western blot analysis, probed with anti-al-3Fucose (upper panel) or anti-IgG1 (lower panel), of crude extracts from plants expressing rituximab, or co-expressing rituximab and RMD. Ritux: rituximab under the control of CPMV 160+; construct number 5072); Ritux+160+/RMD: rituximab under the control of CPMV 160+; construct number 5072, co-expressed with RMD under the control of CPMV 160+; construct number 5093; Ritux+160/RMD: rituximab under the control of CPMV 160+; construct number 5072, co-expressed with RMD under the control of CPMV 160; construct number 5094. OD of each construct at infiltration is indicated between parentheses. Plants were incubated for 7 DPI; 0.25 μg or 0.5 μg of total soluble protein of crude plant extract per lane, as indicated. Anti-Fucose serum (1:10 000) was used to probe for fucose residues. Anti-IgG1 human Jackson Immunoresearch serum (1:7500) was used to probe for rituximab expression.

FIG. 8A shows the nucleotide sequence for primer Flag_Rmd_Fw (SEQ ID NO:19). FIG. 8B shows the nucleotide sequence of primer 5091_5092 IF_Rev (SEQ ID NO:20). FIG. 8C shows the nucleotide sequence of Optimized coding sequence of Pseudomonas aeruginosa RMD from strain PAO1 (SEQ ID NO:21). FIG. 8D shows the nucleotide sequence of primer 5091_IF_Fw (SEQ ID NO:22). FIG. 8E shows a schematic representation of construct 2171. The SacII, AatII and StuI restriction enzyme sites used for plasmid linearization are indicated. FIG. 8F shows the nucleotide sequence of construct 2171 (SEQ ID NO:23; t-DNA borders underlined; 2X35 S/CPMV 160+/NOS with Plastocyanine-P19-Plastocyanine silencing inhibitor expression cassette). Figure G shows the nucleotide sequence of expression cassette number 5091 (SEQ ID NO:24), from the 2X35S promoter to NOS terminator. The RMD (codon optimized) is from Pseudomonas aeruginosa PAO1 strain. Flag-RMD is underlined; FLAG-TAG is annotated in bold. FIG. 8H shows the amino acid sequence (SEQ ID NO:25) of FLAG-Nter-RMD from Pseudomonas aeruginosa PAO1 strain.

FIG. 8I shows a schematic representation of construct number 5091.

FIG. 9A shows the nucleotide sequence for primer 5092 IF_Fw (SEQ NO:26). FIG. 9B shows a schematic representation of construct 1190. The SacII and StuI restriction enzyme sites used for plasmid linearization are indicated. FIG. 9C shows the nucleotide sequence of construct 1190 (SEQ ID NO:27; t-DNA borders underlined; 2X35S/CPMV-160/NOS with Plastocyanine-P19-Plastocyanine silencing inhibitor expression cassette). FIG. 9D shows the nucleotide sequence of expression cassette number 5092 (SEQ ID NO:28) from 2X35S promoter to NOS terminator. RMD (codon optimized) is from Pseudomonas aeruginosa PAO1 strain. Flag-RMD is underlined; FLAG-TAG is annotated in bold. FIG. 9E shows a schematic representation of construct number 5092

FIG. 10A shows the nucleotide sequence of primer 5093 IF_Fw (SEQ ID NO:29). FIG. 10B shows the nucleotide sequence of expression cassette number 5093 (SEQ ID NO:30), from 2X35S promoter to NOS terminator. RMD (codon optimized) from Pseudomonas aeruginosa PAO1 strain is underlined. FIG. 10C shows the amino acid sequence of RMD from Pseudomonas aeruginosa PAO1 strain (SEQ ID NO:31). FIG. 10D shows a schematic representation of construct number 5093

FIG. 11A shows the nucleotide sequence of primer 5094 IF_Fw (SEQ ID NO:32. FIG. 11B shows the nucleotide sequence of expression cassette number 5094 (SEQ ID NO:33), from 2X35S promoter to NOS terminator. RMD (codon optimized) from Pseudomonas aeruginosa PAO1 strain is underlined. FIG. 11C shows a schematic representation of construct number 5094.

FIG. 12A shows the nucleotide sequence of primer IF**(SacII)-PDI.s1+4c (SEQ ID NO:34). FIG. 12B shows the nucleotide sequence of primer IF**-HC(Ritux).s1-6r (SEQ ID NO:35). FIG. 12C shows the nucleotide sequence encoding PDISP/HC rituximab (SEQ ID NO:36). FIG. 12D shows the nucleotide sequence of expression cassette number 2109 (SEQ ID NO:37), from 2X35S promoter to NOS terminator. PDISP/HC rituximab monoclonal antibody is underlined. FIG. 12E shows the amino acid sequence of PDISP/HC rituximab monoclonal antibody (SEQ ID NO:38). FIG. 12F shows the schematic representation of construct number 2109.

FIG. 13 shows the nucleotide sequence of primer IF**-LC(Ritux).s1-6r (SEQ ID NO:39). FIG. 13B shows the nucleotide sequence encoding PDISP/HC rituximab (SEQ ID NO:40). FIG. 13C shows the nucleotide sequence of expression cassette number 2129 (SEQ ID NO:41), from 2X35S promoter to NOS terminator. PDISP/HC rituximab monoclonal antibody is underlined. FIG. 13D shows the amino acid sequence of PDISP/LC rituximab monoclonal antibody (SEQ ID NO:42). FIG. 143E shows a schematic representation of construct number 2129.

FIG. 14A show the nucleotide sequence of expression cassette number 5072 (SEQ ID NO:43), from 2X35S promoter to NOS terminator. PDISP/HC rituximab and PDISP/LC rituximab monoclonal antibody is underlined. FIG. 14B shows a schematic representation of construct number 5072.

FIG. 15A shows the nucleotide sequence of primer IF-atRMD(opt).c (SEQ ID NO:44). FIG. 15B shows the nucleotide sequence of primer IF-atRMD(opt).r (SEQ ID NO:45). FIG. 15C shows the nucleotide sequence encoding optimized Agrobacterium tumefaciens RMD from strain TS43 (SEQ ID NO:46).

FIG. 15D shows the nucleotide sequence of expression cassette number 3431 (SEQ ID NO:47), from 2X35S promoter to NOS terminator. RMD(opt) from Agrobacterium tumefaciens strain TS43 is underlined. FIG. 15E shows the amino acid sequence of RMD from Agrobacterium tumefaciens strain TS43 (SEQ ID NO:48). FIG. 15F shows a schematic representation of construct number 3431.

FIG. 16A shows the nucleotide sequence of primer IF-pbRMD(opt).c (SEQ ID NO:49). FIG. 16B shows the nucleotide sequence of primer IF-pbRMD(opt).r (SEQ ID NO:50). FIG. 16C shows the nucleotide sequence encoding optimized Pseudomonas brassicacearum RMD from strain NFM421 (SEQ ID NO:51). FIG. 16D shows the nucleotide sequence of expression cassette number 3432 (SEQ ID NO:52), from 2X35S promoter to NOS terminator. RMD(opt) from Pseudomonas brassicacearum strain NFM421 is underlined. FIG. 16E shows the amino acid sequence of RMD from Pseudomonas brassicacearum strain NFM421 (SEQ ID NO:53). FIG. 16F shows a schematic representation of construct number 3432.

FIG. 17A shows the nucleotide sequence of primer IF-psRMD(opt).c (SEQ ID NO:54). FIG. 17B shows the nucleotide sequence of primer IF-psRMD(opt).r (SEQ ID NO:55). FIG. 17C shows the nucleotide sequence encoding optimized Pseudomonas syringae RMD (SEQ ID NO:56). FIG. 17D shows the nucleotide sequence of expression cassette number 3433 (SEQ ID NO:57), from 2X35S promoter to NOS terminator. RMD(opt) from Pseudomonas syringae is underlined. FIG. 17E shows the amino acid sequence of RMD from Pseudomonas syringae (SEQ ID NO:58). FIG. 17F shows a schematic representation of construct number 3433.

FIG. 18A shows the nucleotide sequence of primer IF-xvRMD(opt).c (SEQ ID NO:59). FIG. 18B shows the nucleotide sequence of primer IF-xvRMD(opt).r (SEQ ID NO:60). FIG. 18C shows the nucleotide sequence encoding optimized Xanthomonas vasicola RMD from strain NCPPB 1326 (SEQ ID NO:61). FIG. 18D shows the nucleotide sequence of expression cassette number 3434 (SEQ ID NO:62), from 2X35S promoter to NOS terminator. RMD(opt) from Xanthomonas vasicola strain NCPPB 1326 is underlined. FIG. 18E shows the amino acid sequence of RMD from Xanthomonas vasicola strain NCPPB 1326 (SEQ ID NO:63). FIG. 18F shows a schematic representation of construct number 3434.

### DETAILED DESCRIPTION

The present invention relates to methods for modifying glycoprotein production in plants using GDP-4-dehydro-6-deoxy-D-mannose reductase (RMD). The present invention also provides plants with modified glycoprotein production.

A method is provided for the production of a protein of interest in a plant, a portion of the plant, or a plant cell, wherein upon expression, the protein of interest comprises N-glycans having a modified N-glycosylation profile, for example with reduced fucosylated N-glycans. Furthermore, the protein of interest may have reduced, or lack, fucosylated N-glycans, xylosylated N-glycans, or both fucosylated and xylosylated N-glycans.

Furthermore, a method is provided for producing a protein of interest comprising N-glycans characterized as having a reduced fucose content in a plant, a portion of a plant, or a plant cell, where the plant, the portion of the plant, or the plant cell have reduced fucosylation activity. The method involves co-expressing within the plant, the portion of the plant, or the plant cell having reduced fucosylation activity, a nucleotide sequence encoding a first nucleotide sequence encoding a GDP-4-dehydro-6-deoxy-D-mannose reductase (RMD) the first nucleotide sequence operatively linked with a first regulatory region that is active in the plant, the portion of a plant, or the plant cell, and a second nucleotide sequence encoding the protein of interest, the second nucleotide sequence operatively linked with a second regulatory region that is active in the plant, the portion of a plant, or the plant cell, and co-expressing the first and second nucleotide sequences to synthesize a protein of interest comprising N-glycans with a reduced fucose content, when compared to the fucose content of the protein of interest expressed in the plant, the portion of the plant, or the plant cell that does not express RMD.

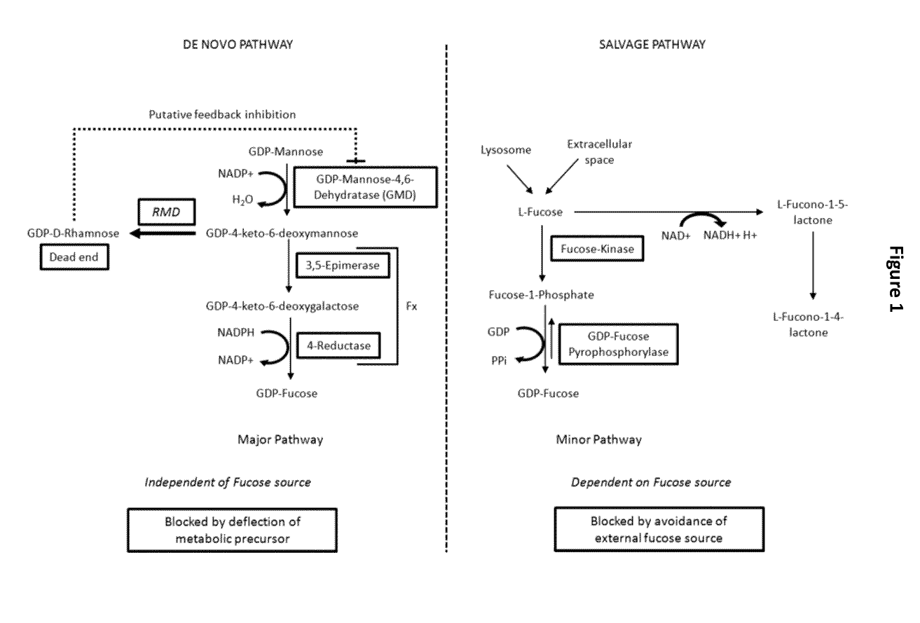

The methods described herein involve co-expressing the protein of interest along with GDP-6-deoxy-D-lyxo-4-hexulose reductase (synonymous with GDP-4-keto-6-deoxy-D-mannose reductase, or RMD) in a plant, portion of a plant, or plant cell. RMD transforms GDP-4-keto-6-deoxy-D-mannose, which is a precursor or GDP-L-fucose, into GDP-D-Rhamnose which cannot be utilized for N-glycan modification in plants (see FIG. 1). Without wishing to be bound by theory, it is believed that the expression of RMD in plant indirectly blocks glycoprotein fucosylation by blocking the production of GDP-L-fucose, which is required as the donor substrate for this process. Furthermore, GDP-D-Rhamnose is believed to be a dead-end product in plants.

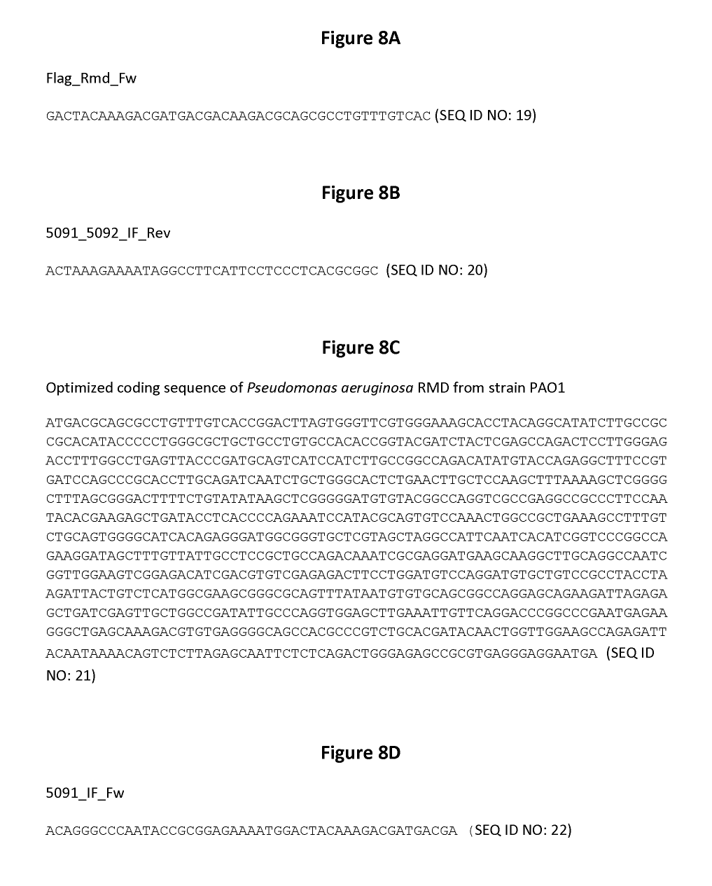

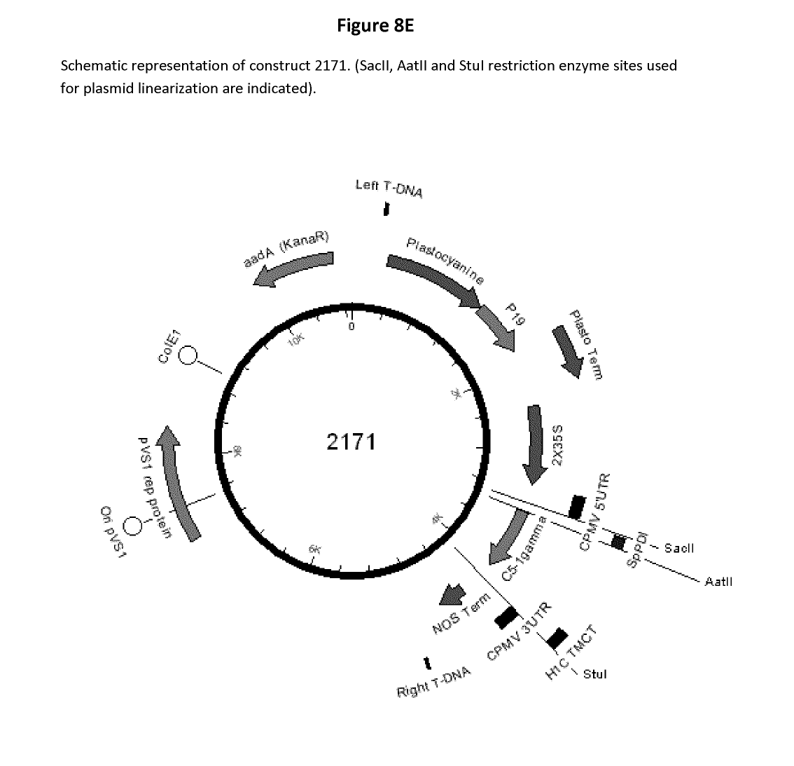

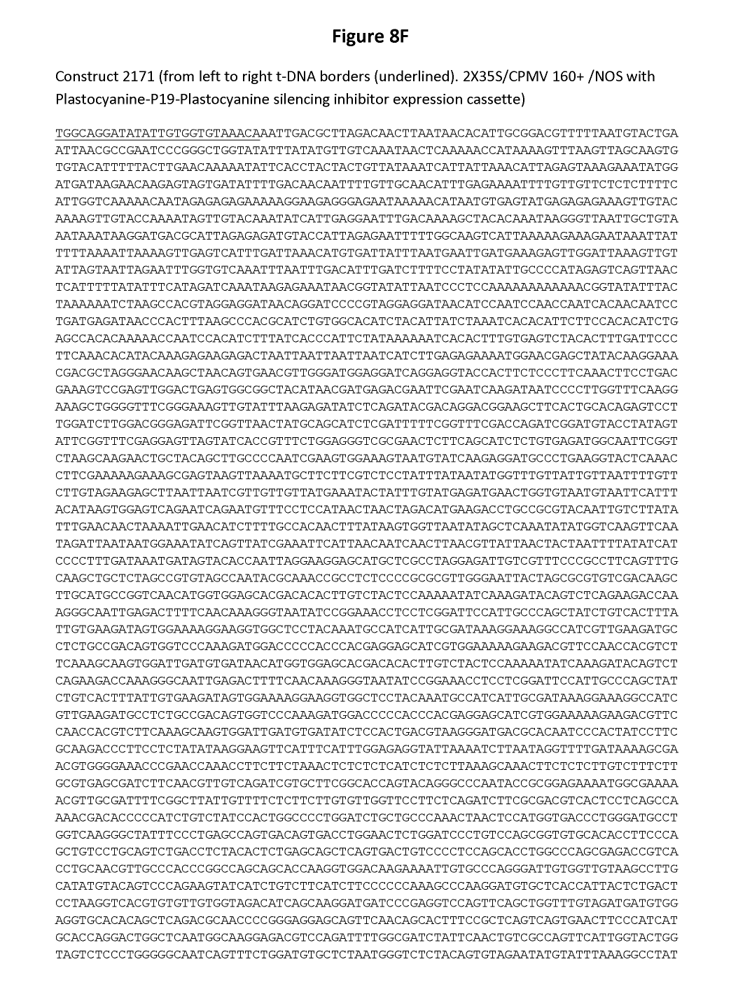

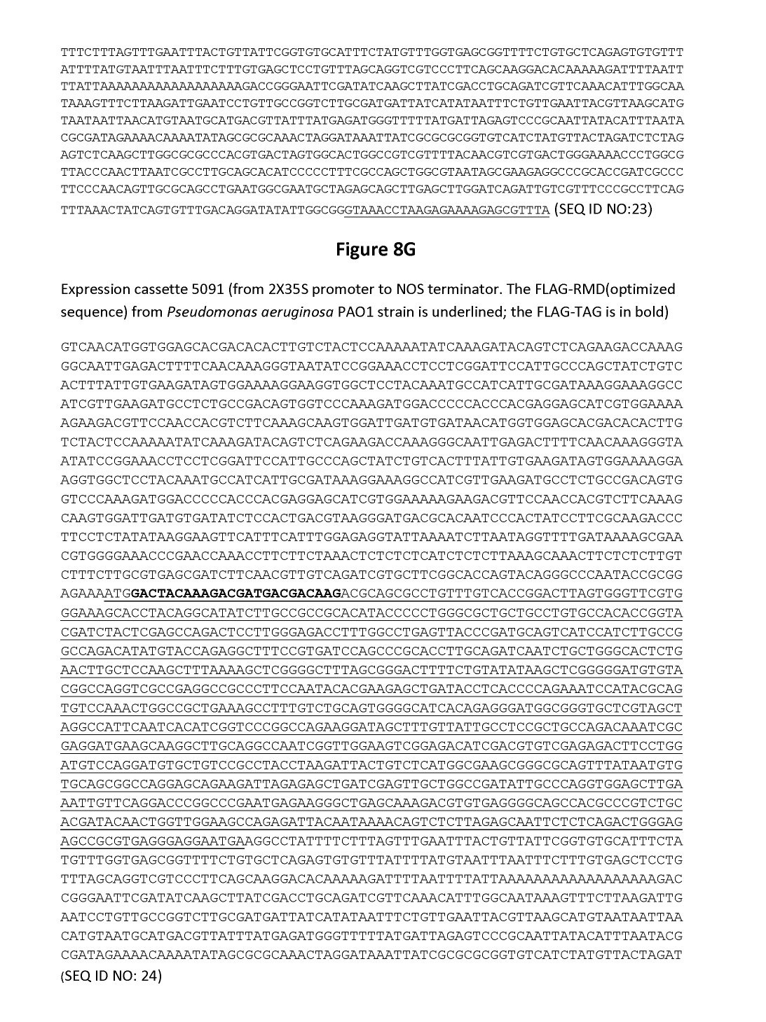

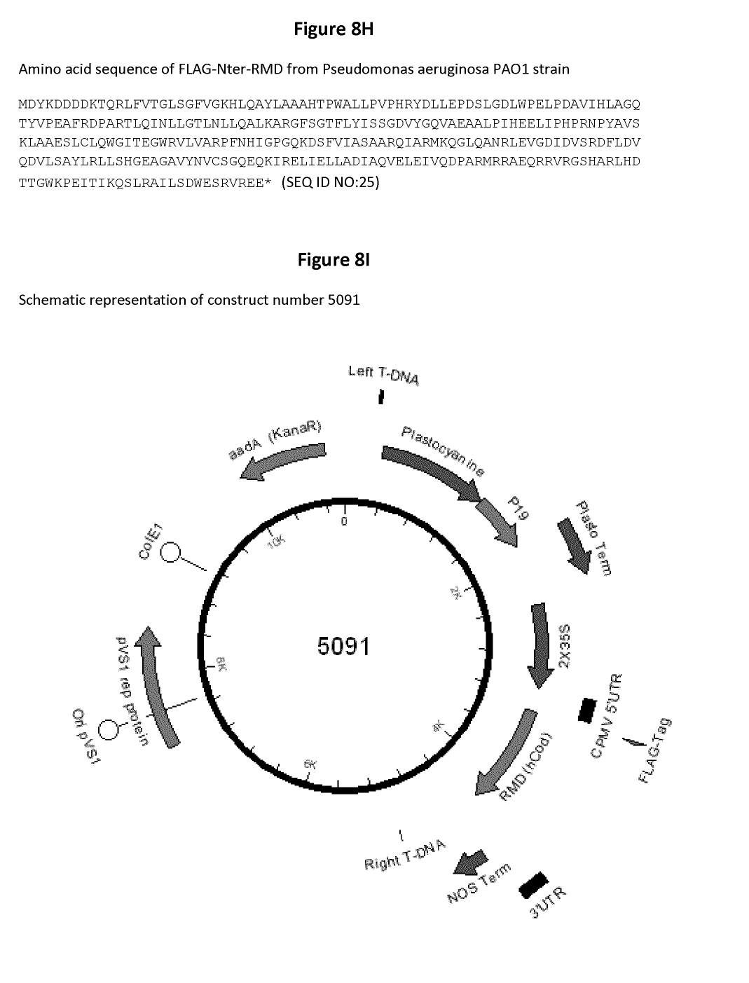

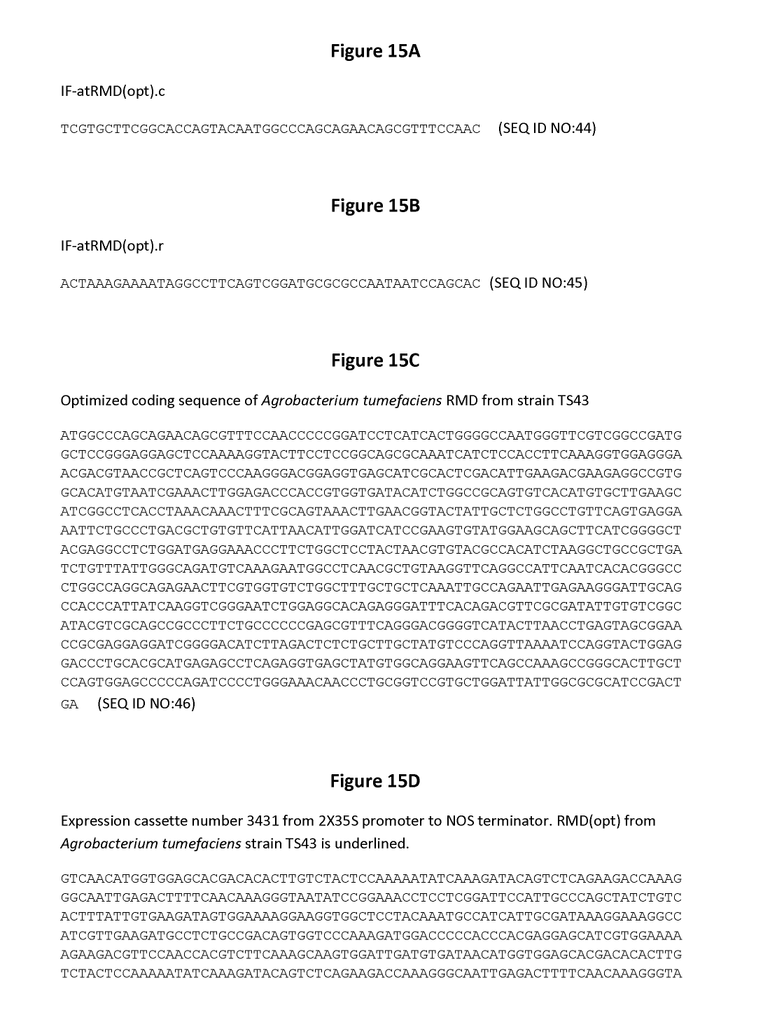

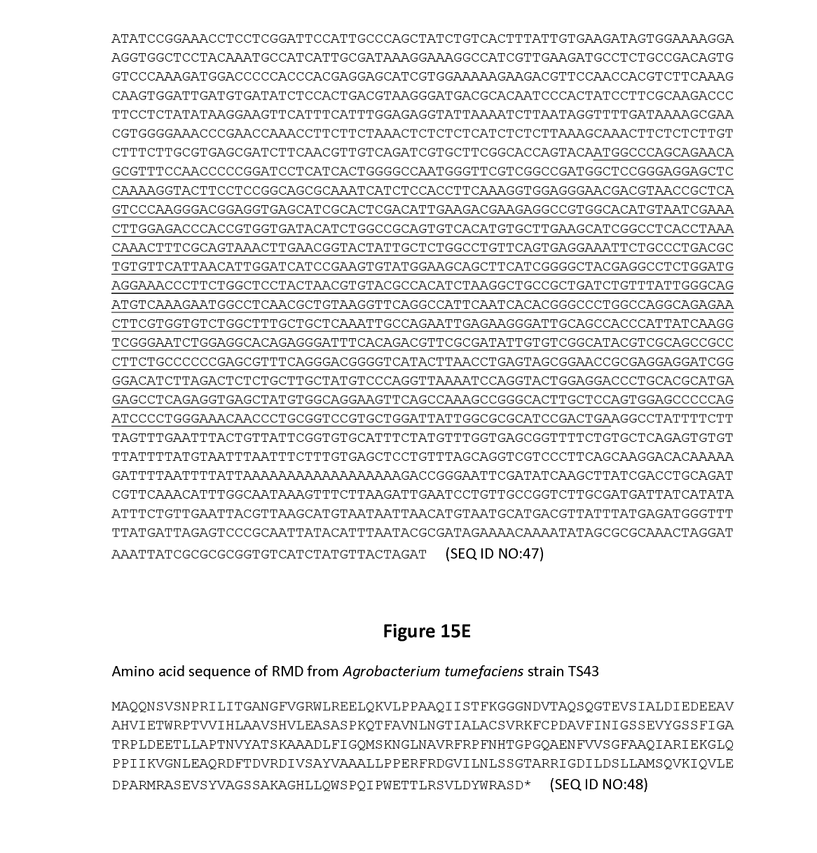

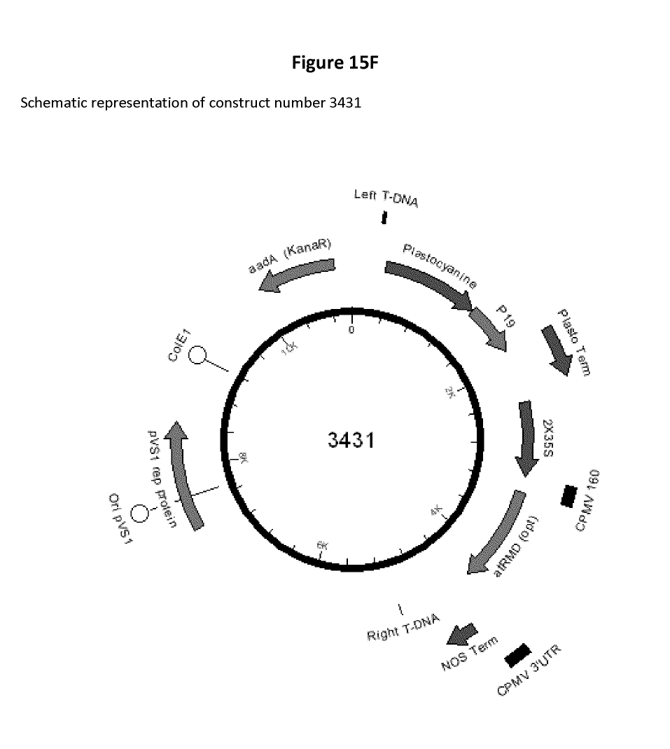

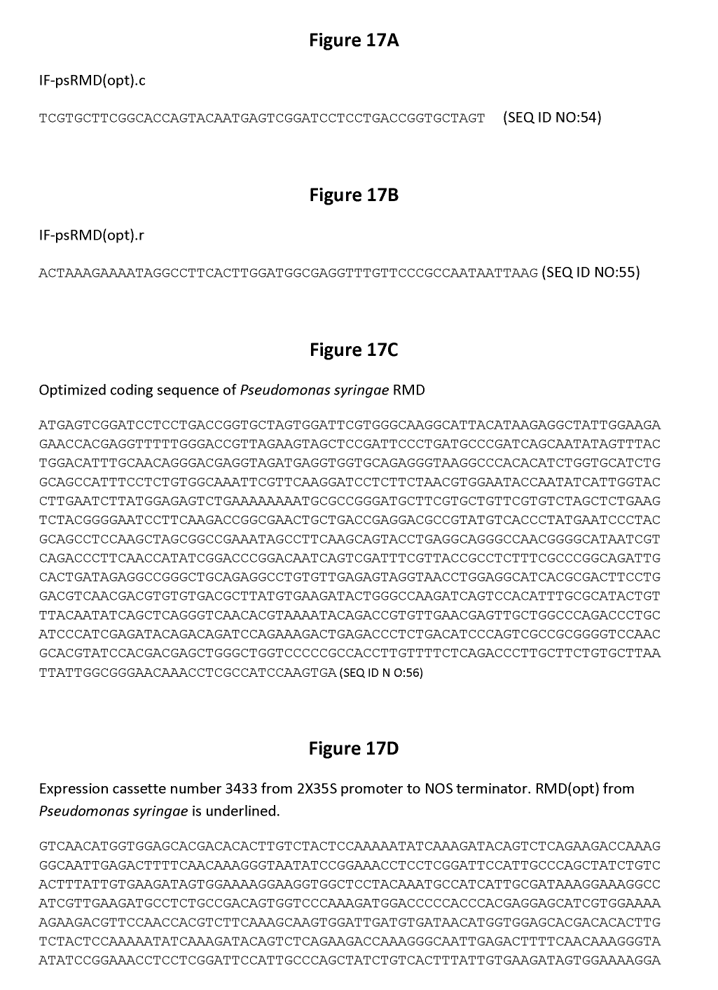

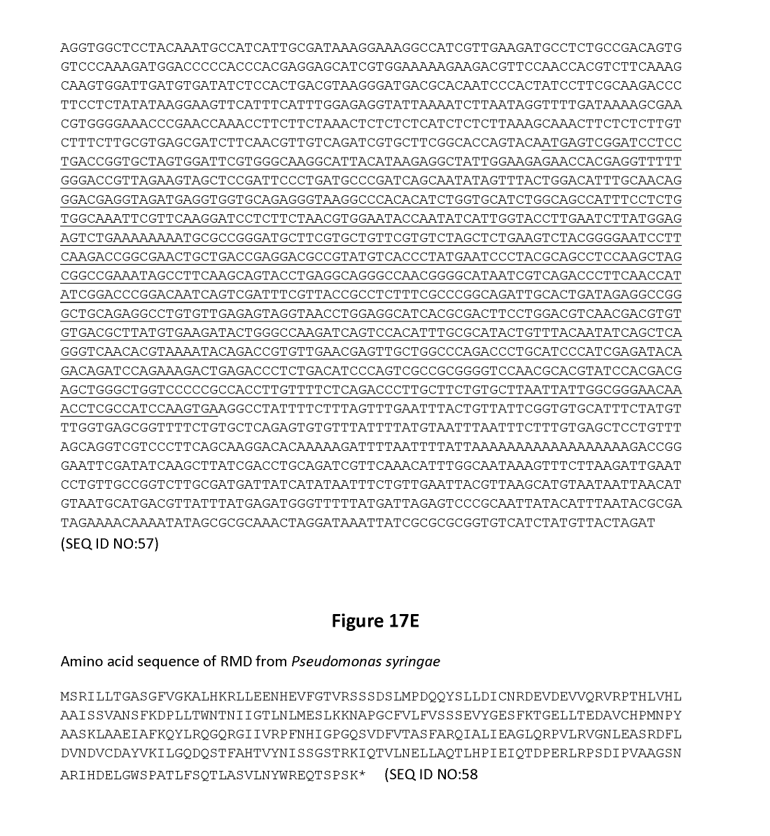

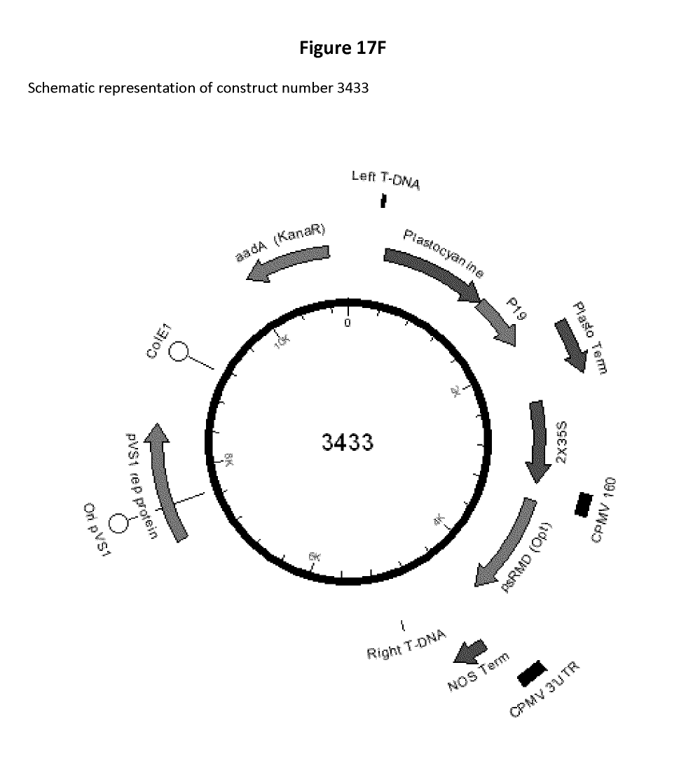

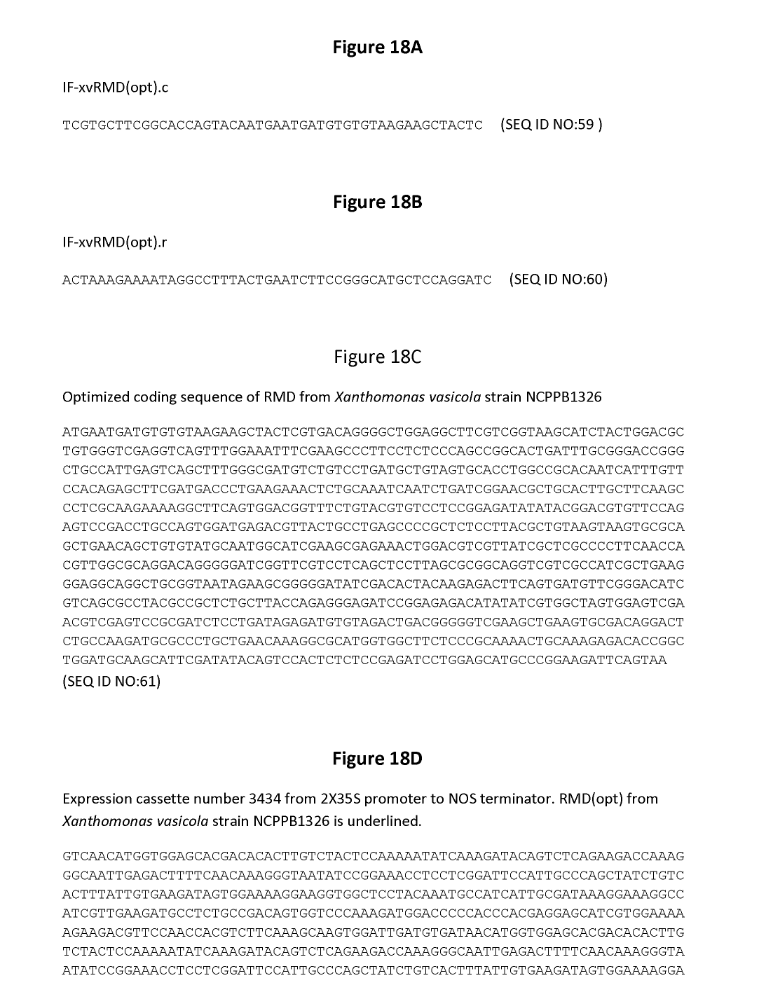

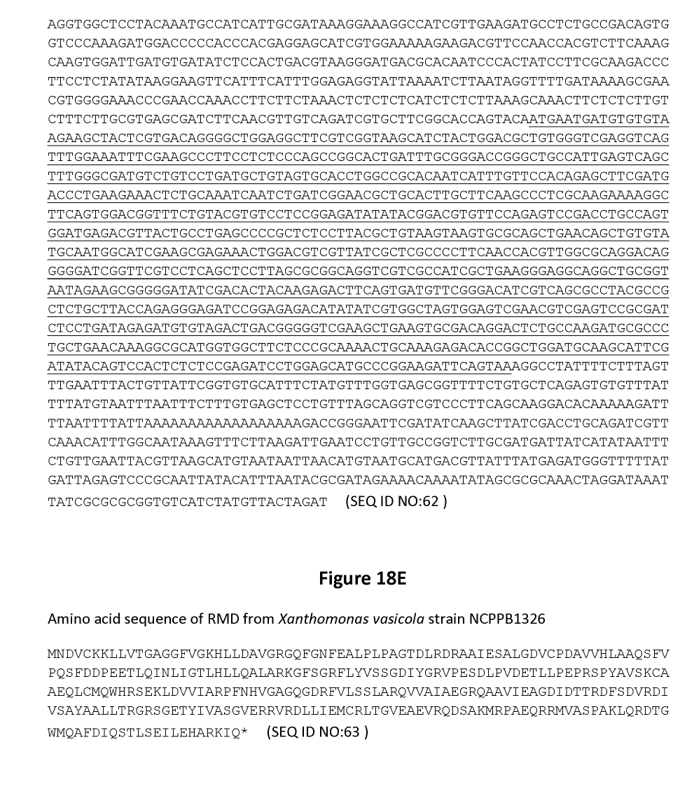

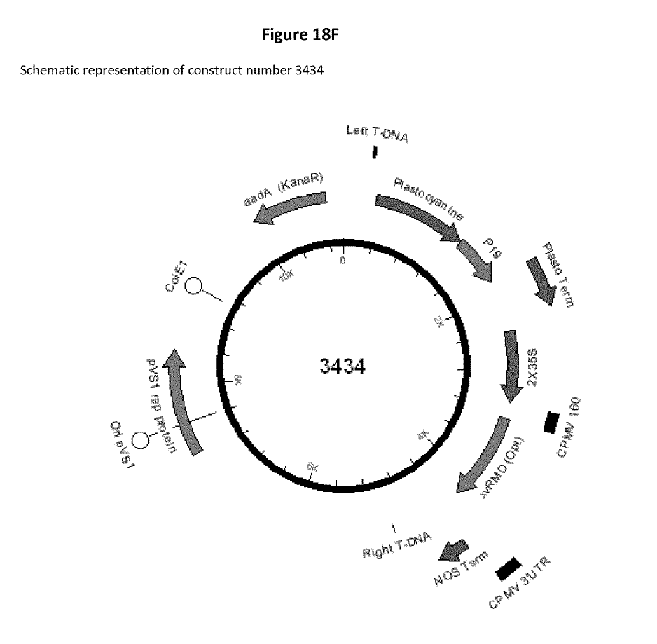

The RMD used in the methods described herein, may be a bacterial RMD (EC 1.1.1.281) and may be derived from any source, for example, but not limited to a bacterial source, for example, Pseudomonas aeruginosa (Maki, M. et. al., 2002, Eur. J. Biochem. 269 (2): 593-601, which is incorporated herein by reference), Agrobacterium tumefaciens (Watt et. al., Plant Physiol. 2004 April; 134(4): 1337-1346, which is incorporated herein by reference), E. coli (Rizzi M., et. al., Structure. 1998 Nov. 15; 6(11):1453-65, which is incorporated herein by reference), Aneurinibacillus thermoaerophilus L420-91T (Messner, P. et. al., J. Biol. Chem. 276 (8): 5577-83, which is incorporated herein by reference), or other bacteria from Pseudomonas spp. and Xanthomonas spp. For example, the bacterial RMD may be obtained from Pseudomonas aeruginosa (SEQ ID NO:21; FIG. 8C), Pseudomonas syringae (SEQ ID NO:56; FIG. 17C), Pseudomonas brassicearum (SEQ ID NO:51; FIG. 15C), Agrobacterium tumefaciens (SEQ ID NO:46; FIG. 15C), or Xanthomonas vasicola (SEQ ID NO:61; FIG. 18C).

In one embodiment the plant, portion of the plant, or plant cell, expressing RMD maybe a plant, portion of a plant, or plant cell that exhibits reduced α(1,3)-fucosyltransferase (FucT) activity, reduced β(1,2)-xylosyltransferase (XylT) activity, or reduced FucT and XylT (FucT/XylT) activity. Interruption of FucT, or FucT and XylT function may be achieved by well-known methods in the art. For example the FucT gene, or the FucT and XylT genes may be knocked out as described in Li et al. (Plant Biotechnology Journal Volume 14, Issue 2 Feb. 2016, which is incorporated herein by reference) or as described in WO 2014/071039 and US 2015/0272076 (which are herein incorporated by reference). Interruption of FucT gene, XylT gene, or both FucT and XylT gene functions also be achieved using RNA interference (RNAi) technology, random mutagenesis or other well-known methods in the art. Chemical inhibition of FucT activity may be achieved using one or more chemical inhibitors, for example, which is not to be considered limiting, by treating the plant or portion of the plant may with 2F-Peracetyl-Fucose (a cell-permeable fluorinated fucose derivative that acts as an inhibitor of FucT following uptake and metabolic transformation into a GDPfucose mimetic), stachybotrdial (a spirocyclic drimane isolated from Stachybotrys cylindrospora; Tzu-Wen et. al., 2005, BBRC 331:953-957), or other known inhibitors of FucT activity (see Merino P. et. al., 2012, Mini Rev Med Chem. December; 12(14):1455-64; Tu Z. et. al., 2013, Chem Soc Rev. May 21; 42(10):4459-75).

In another embodiment, the plant, portion of the plant, or plant cell may further comprise a hybrid protein or hybrid enzyme as for example described below. For example in these plants the protein of interest may be co-expressed with the hybrid enzyme and RMD. Furthermore, the protein of interest may be co-expressed with the hybrid enzyme and RMD in plants, portion of plants, or plant cells that exhibit reduced, or that lack, FucT activity, or FucT and XylT activity as described herein.

If plant protoplasts, or a plant cell system, is used for the methods as described herein then the fucose salvage pathway may be blocked within the plant cell. For example, growth media free of fucose and of fucosylated glycoproteins, may be used when culturing plant protoplasts or plant cells expressing the proteins of the present invention. Any plant may be used according to the methods described herein. For example but not limited to, tobacco, Nicotiana spp., N. benthamiana, alfalfa, soybean, sunflower, potato, canola, Brassica spp., cotton, wheat, corn, maize, oat, rice, barley.

The salvage pathway may be blocked by additionally co-expressing L-fucose dehydrogenase in the plant, portion of the plant, or plant cell. L-fucose dehydrogenase converts L-Fucose into L-Fucono-1,5-lactone (see FIG. 1). The L-fucose dehydrogenase may be obtained from any suitable source, for example but not limited to Agrobacterium tumefaciens (protein accession number WP_010973342).

By “co-expressed” it is meant that two or more than two nucleotide sequences are expressed at about the same time within the plant, and within the same tissue of the plant. For example, two, three, four or more nucleotide sequences may be expressed at about the same time within the plant, plant portion or plant cell. However, the nucleotide sequences need not be expressed at exactly the same time. Rather, the two or more nucleotide sequences may be expressed in a manner such that the encoded products have a chance to interact when expressed within the plant, plant portion or plant cell. For example, RMD may be expressed either before or during the period when the protein of interest is expressed so that modification of the glycosylation of the protein of interest takes place. The two or more nucleotide sequences can be co-expressed using a transient expression system, where the two or more sequences are introduced within the plant at about the same time under conditions that both sequences are expressed. Alternatively, a platform plant comprising one of the nucleotide sequences, for example the sequence encoding RMD, may be transformed either transiently or in a stable manner with an additional sequence encoding the protein of interest. In this case, the sequence encoding RMD may be expressed within a desired tissue, during a desired stage of development, or its expression may be induced using an inducible promoter, and the additional sequence encoding the protein of interest may be expressed under similar conditions and in the same tissue, to ensure that the nucleotide sequences are co-expressed.

The terms “glycan” or “glycomoiety” are used interchangeably in the context of the present invention and they refer to a polysaccharide or oligosaccharide. The term “oligosaccharide” means a saccharide polymer containing a small number (typically three to ten) of component sugars, also known as simple sugars or monosaccharides. The term “polysaccharide” means a polymeric carbohydrate structure, formed of repeating units (either mono- or disaccharides, typically greater than 10 repeating units) joined together by glycosidic bonds. Glycans can be found attached to proteins as in glycoproteins or attached to lipids as in glycolipids. The terms “glycan” or “glycomoiety” encompass N-glycans, such as high mannose type N-glycans, complex type N-glycans, or hybrid type N-glycans or O-glycans.

By “N-glycosylation” it is meant the addition of sugar chains which to the amide nitrogen on the side chain of asparagine. “O-glycosylation” means the addition of sugar chains on the hydroxyl oxygen on the side chain of hydroxylysine, hydroxyproline, serine, or threonine. An “N-glycan” means an N-linked polysaccharide or oligosaccharide. An N-linked oligosaccharide is for example one that is or was attached by an N-acetylglucosamine residue linked to the amide nitrogen of an asparagine residue in a protein

In the context of the present invention, the following glycans are abbreviated as follows:

Glucose: Glc;

Galactose: Gal or G;

Mannose: Man or M;

Fucose: Fuc or F;

N-acetylgalactosamine: GalNAc:

N-acetylglucosamine: GlcNAc or Gn;

Xylose: Xyl or X.

By “modified glycosylation” of a protein of interest it is meant that the N-glycan profile of the protein of interest is altered from that of the N-glycan profile of the protein of interest produced in a wild-type plant. Modification of glycosylation may include an increase or a decrease in one or more than one glycan of the protein of interest, or the bisecting of GlnAc. For example, the protein of interest may exhibit reduced fucosylation, reduced xylosylation, or both reduced fucosylation and xylosylation, for example the protein of interest may lack or may have reduced amounts of Gn2M3XGn2, Gn2M3FGn2, Gn2M3XFGn2 type N-glycans or a combination thereof. For example, the protein of interest may comprise a modified glycosylation profile comprising from about 0-48%, or any amount therebetween, of N-glycans comprising α(1,3)-fucose in the form: Gn2M3FGn2 and Gn2M3XFGn2 (compared to the wild type glycosylation profile of a protein of interest that comprises from 70%-80% of α(1,3)-fucose in the form: Gn2M3XGn2 and Gn2M3XFGn2; see Tables 5-9 in the Examples below). For example, the protein of interest may comprise a modified glycosylation profile comprising from about 0, 2, 4, 6, 8, 10, 12, 14, 16, 18, 20, 22, 24, 26, 28, 30, 32, 34, 36, 38, 40, 42, 44, 46, 48%, or any amount therebetween, of N-glycans comprising α(1,3)-fucose in the form: Gn2M3FGn2 and Gn2M3XFGn2 (compared to the wild type glycosylation profile of a protein of interest that comprises from 70%-80% of α(1,3)-fucose in the form: Gn2M3XGn2 and Gn2M3XFGn2). The protein of interest may comprise a modified glycosylation profile comprising from about 9-70%, or any amount therebetween, of N-glycans comprising α(1,3)-fucose in the form: Gn2M3FGn2 and Gn2M3XFGn2 (compared to the wild type glycosylation profile of a protein of interest that comprises 80%-85% of α(1,3)-fucose in the form: Gn2M3XGn2 and Gn2M3XFGn2; see Tables 5-9 in the Examples below). For example, the protein of interest may comprise a modified glycosylation profile comprising from about 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70%, or any amount therebetween, of N-glycans comprising α(1,3)-fucose in the form: Gn2M3FGn2 and Gn2M3XFGn2 (compared to the wild type glycosylation profile of a protein of interest that comprises 80%-85% of α(1,3)-fucose in the form: Gn2M3XGn2 and Gn2M3XFGn2.

Furthermore, the N-glycan profile of the protein of interest may be modified in a manner so that the amount of Gn2M3Gn2 type N-glycans is increased, and optionally, the amount fucosylation in the glycosylated protein of interest is reduced. For example, the protein if interest may comprise a modified glycosylation profile comprising from about 15-91%, or any amount therebetween, of N-glycans comprising Gn2M3Gn2 (compared to the wild type glycosylation profile of a protein of interest that comprises 4-6% of Gn2M3Gn2; see Tables 5-7 in the Examples below).

By “reduced fucosylation” of a protein of interest, it is meant that the amount of fucosylation of N-glycans detectable on the protein of interest is less than 10% of that of the amount fucosylation that is detectable on the protein of interest when produced within a wild-type plant, and where the protein of interest is isolated, and where fucosylation is determined, using the same method (i.e. a 10% reduction in the amount of fucosylation when compared to the wild-type protein). For example, the protein of interest may comprise a reduction of from about 10% to about 100%, or any amount therebetween, of the N-glycan residues that are fucosylated, when compared to the same protein of interest produced in a wild-type plant (or conversely, the protein of interest may comprise from about 0% to about 90%, or any amount therebetween, fucosylated N-glycan residues, when compared to the same protein of interest produced in a wild-type plant). For example, the protein of interest may comprise a reduction of from about 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100%, or any amount therebetween, of the N-glycan residues that are fucosylated, when compared to the same protein of interest produced in a wild-type plant A protein of interest may therefore be produced in high yield and lack glycans that may provoke hypersensitivity reactions, or be otherwise involved in allergenic reactions.

Reduced fucolsylation activity, or reduced α(1,3)-fucosyltransferase (FucT) activity, may be achieved by interrupting expression of the FucT gene for example by knocking out the gene (WO 2014/071039; US 2015/0272076, or Li et. al., 2015, Plt. Biotech. J., pp. 1-10), using RNA interference (RNAi) technology, transient expression of an RNAi construct, random mutagenesis, or by chemically inhibiting FucT activity. Chemical inhibitors of FucT activity may include 2F-Peracetyl-Fucose, stachybotrdial (Tzu-Wen et. al., 2005, BBRC 331:953-957), or other known FucT inhibitors as identified in Merino P. et. al. (2012, Mini Rev Med Chem. December; 12(14):1455-64; Tu Z. et. al., 2013, Chem Soc Rev. May 21; 42(10):4459-75). For example, fucosylation may be reduced from about 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 95%, or any amount therebetween, when compared to wild type fucosylation activity in the same plant.

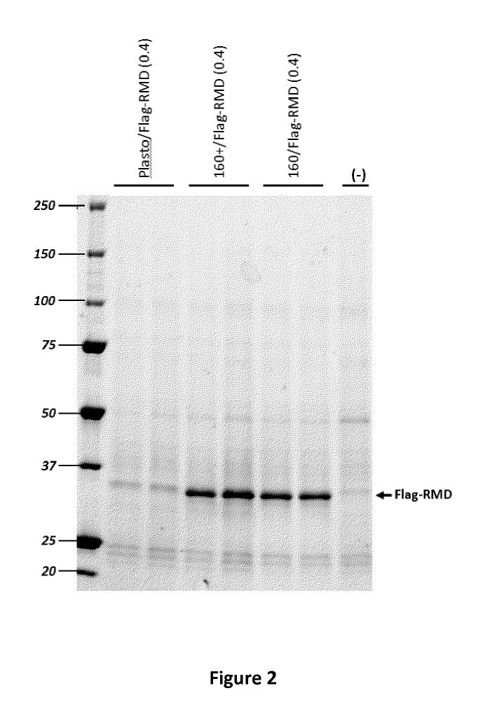

When a protein of interest was co-expressed with RMD in wild-type plants, and RMD expression was under the control of an expression enhancer, for example, a CPMV-derived expression enhancer as described below, RMD expression was observed (see FIG. 2, 160+/Flag-RMD; construct 5091, and 160/Flag-RMD; construct 5092). However, when RMD was expressed under the control of a plastocyanin promoter (a constitutive promoter/regulatory element), no accumulation of RMD protein was observed (see FIG. 2; plasto/Flag-RMD; construct 1191). As a result, an expression enhancer was used to drive RMD expression in plants.

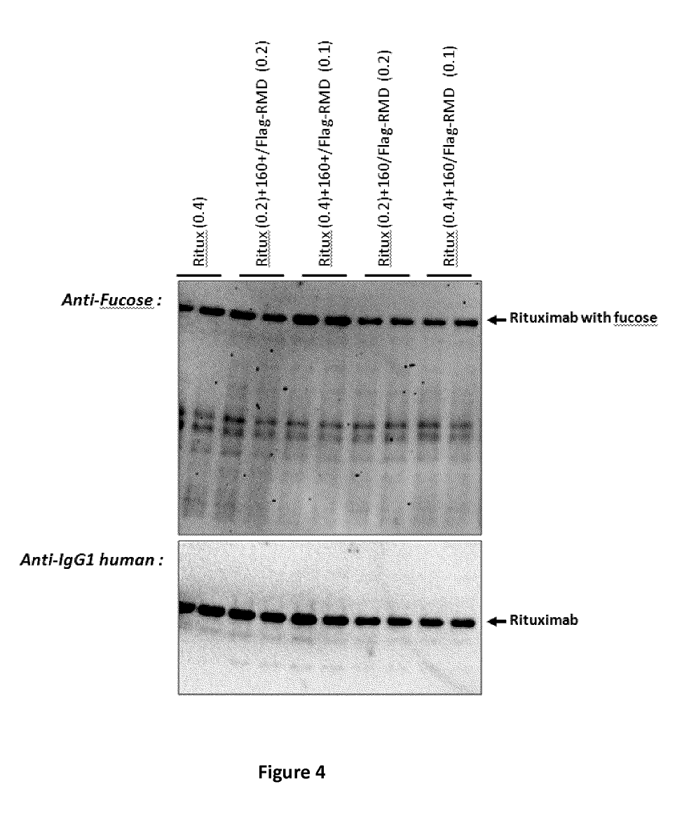

As described below, reduced fucosylation of the protein of interest was observed when RMD, for example paRMD, was expressed under the control of an expression enhancer, for example but not limited to CPMV HT, CPMV HT+, CPMV160+ or CPMV160. As shown in tables 3 and 4, fucosylation of a protein of interest (determined by Western analysis), for example, but not limited rituximab (also termed C2B8, a chimeric (mouse/human) monoclonal antibody directed against the B-cell-specific antigen CD20 expressed on non-Hodgkin's lymphomas; NHL)., co-expressed with 160+/RMD (also referred to as 160+/paRMD) in wild-type plants, was reduced by about 65% to about 76% (or about 37 to about 53% with 160/RMD, also referred to 160/paRMD) when compared to rituximab expressed alone in the same plant. Similar results are observed with other glycoproteins, for example IgG, HA and other proteins that are co-expressed with RMD under the control of an expression enhancer. Preferably, the RMD sequence does not comprise a “Flag” sequence (see Example 2; FIG. 4).

Fucosylation of N-glycans within a protein of interest (determined by MS analysis), for example but not limited to rituximab, was also reduced when co-expressed with RMD, for example, but not limited to paRMD, atRMD, pbRMD, psRMD, or xvRMD. As shown in Tables 5 to 9, by using the methods described herein, a protein of interest may be produced that exhibits a modified glycosylation profile. For example, a protein of interest which comprises glycans with reduced levels of fucose residues, and increased levels of desirable Gn2M3Gn2, has been produced when the protein of interest is co-expressed with RMD. For example the protein of interest may have zero levels of Gn2M3FGn2 type glycans and reduced levels of Gn2M3XFGn2 type glycans. For example, from 75-92% of N-glycans of rituximab expressed alone in wild-type plants had an α(1,3)-fucose (Tables 5-7, Example 3). However, in plants co-expressing 160+/paRMD, about 61 to about 81% of the N-glycans did not have α(1,3)-fucose (i.e. about 60 to about 80% reduction in the amount of fucosylation when compared with the amount of fucosylation observed in wild-type plants). For plants co-expressing rituximab and 160/paRMD, about 50 to about 66% of the N-glycans did not have α(1,3)-fucose (about 44 to about 50% reduction in the amount of fucosylation when compared with the amount of fucosylation observed in wild-type plants). Similar results in the reduction of fucosylation of a protein of interest (rituximab), when co-expressed with atRMD, pbRMD, psRMD or xvRMD was also observed (see Table 8 and 9, Example 4).

Similar results were also observed with other glycoproteins, for example IgG, HA and other proteins, that were co-expressed with paRMD under the control of an expression enhancer.

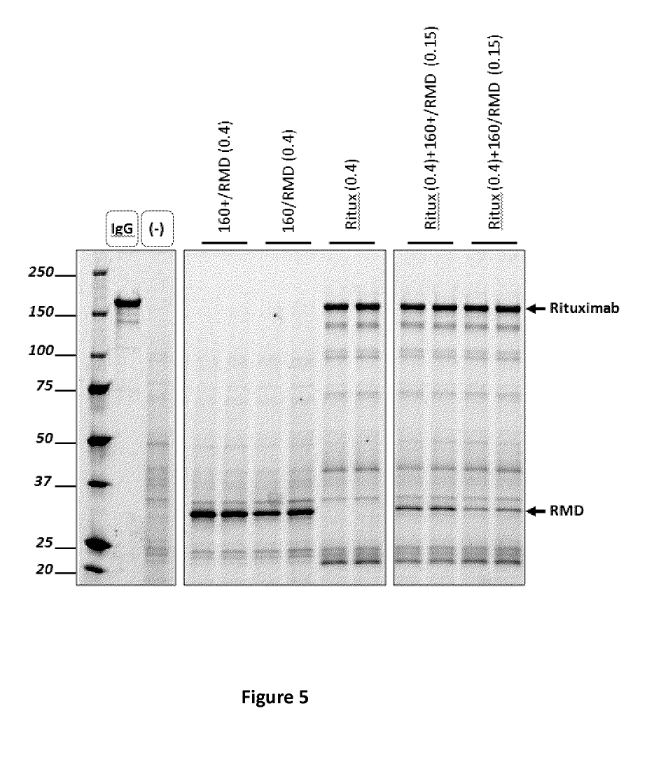

These results are to be contrasted with those observed in CHO cells co-expressing RMD as described in U.S. Pat. No. 8,642,292. Co-expression of an IgG with RMD under the control of a constitutive promoter, in the absence of an expression enhancer, in CHO cells resulted in a 98% reduction in fucosylation of the protein and only trace amounts (up to 2% of the glycan pool) of fucose was observed in IgG samples (determined using MALDI-TOF/TOF; see FIGS. 5A-D of U.S. Pat. No. 8,642,292).

In order to determine if a further reduction of fucosylation of a protein of interest may be obtained in a plant, FucT knock out plants were used as hosts for the co-expression of a protein of interest along with RMD. Co-expression of the protein of interest with RMD in a plant that has reduced FucT activity (FucT knockout), for example NB13-105a or NB13-213a (Li et. al. 2015, Plt. Biotech. J. p 1-10; which is incorporated herein by reference), or XylT and FucT activity (FucT/XylT knock-out plants), for example NB14-29aT2 as described in WO 2014/071039; US 2015/0272076; Li et. al. (2015, Plant Biotech. J., pp. 1-10; each of which are herein incorporated by reference), resulted in an increase in the levels of desirable Gn2M3Gn2 type glycans, and in reduced levels of fucose, in the form or the N-glycans Gn2M3FGn2 and Gn2M3XFGn2 in the protein of interest. Furthermore, using FucT/XylT knockout plants that co-express RMD and a protein of interest, the protein of interest exhibits reduced or no xylose content, for example a protein of interest having no Gn2M3XGn2 and Gn2M3XFGn2 N glycans. As shown in Tables 6 and 7 (Example 3), glycosylation analysis of FucT/XylT knockout plants (NB14-29aT2) resulted in a reduction of fucosylation, of about 88% when rituximab was co-expressed with 160+/RMD and from about 63 to about 89%, when co-expressed with varying amounts of 160/RMD. Similar results in the reduction of fucosylation of a protein of interest (rituximab), when co-expressed with atRMD, pbRMD, psRMD or xvRMD was also observed (see Table 8 and 9, Example 4).

Similar results of reduced fucose content were also observed when other glycoproteins, for example IgG, and HA were co-expressed with paRMD.

The modulation in the amount of fucosylation may be determined using any suitable method, for example using anti-alpha-1,3fucose antibodies (western analysis), to detect the presence or absence of fucose-specific immunosignals (fucosylation). Alternatively, LC ESI MS/MS (mass spectrometry) analysis of glycopeptides as described in Li et. al. (2015, Plant Biotech. J., pp. 1-10) may be used to determine the N glycosylation profile of a protein or a portion of the protein. Other method to determine the N-glycan profile of a protein or portion of the protein known to one of skill in the art may also be used.

Therefore, the present invention provides a method of producing a protein of interest comprising N-glycans with modified N-glycosylation profile in a plant comprising co-expressing within a plant, a portion of a plant, or a plant cell, a nucleotide sequence encoding a first nucleotide sequence encoding a GDP-4-dehydro-6-deoxy-D-mannose reductase (RMD) the first nucleotide sequence operatively linked with a first regulatory region that is active in the plant, and a second nucleotide sequence for encoding the protein of interest, the second nucleotide sequence operatively linked with a second regulatory region that is active in the plant, and co-expressing the first and second nucleotide sequences to synthesize a protein of interest comprising glycans with the modified N-glycosylation profile.

The plant, portion of the plant, or plant cell may further exhibit reduced α(1,3)-fucosyltransferase (FucT) activity, for example, but not limited to NB13-105a and NB13-213a (FucT knockout plants), or NB14-29at2 (FucT/XulT knock out plants; Li. Et. al., 2015 Plant Biotech. J., pp. 1-10). Alternatively, FucT activity may be reduced using RNA interference (RNAi), random mutagenesis, or by chemically inhibiting FucT activity. Chemical inhibitors of FucT activity may include 2F-Peracetyl-Fucose, stachybotrdial (Tzu-Wen et. al., 2005, BBRC 331:953-957), or other known FucT inhibitors as identified in Merino P. et. al. (2012, Mini Rev Med Chem. December; 12(14):1455-64; Tu Z. et. al., 2013, Chem Soc Rev. May 21; 42(10):4459-75).

The protein of interest so produced may be recovered from the plant. Furthermore, the protein of interest may be partially purified of purified using standard techniques as would be known to one of skill in the art.

By “gene of interest”, “nucleotide sequence of interest”, or “coding region of interest”, it is meant any gene, nucleotide sequence, or coding region that is to be expressed within a host organism, for example a plant. These terms are used interchangeably. Such a nucleotide sequence of interest may include, but is not limited to, a gene or coding region whose product is a protein of interest. Examples of a protein of interest include, for example but not limited to, an industrial enzyme, a protein supplement, a nutraceutical, a value-added product, or a fragment thereof for feed, food, or both feed and food use, a pharmaceutically active protein, for example but not limited to growth factors, growth regulators, antibodies, antigens, autoantigens, glycoproteins, artificial glycoproteins, and fragments thereof, or their derivatives useful for immunization or vaccination and the like. Additional proteins of interest may include, but are not limited to, interleukins, for example one or more than one of IL-1 to IL-24, IL-26 and IL-27, cytokines, Erythropoietin (EPO), insulin, G-CSF, GM-CSF, hPG-CSF, M-CSF or combinations thereof, interferons, for example, interferon-alpha, interferon-beta, interferon-gamma, blood clotting factors, for example, Factor VIII, Factor IX, or tPA hGH, receptors, receptor agonists, antibodies, for example IgG1, IgG2, IgA, IgM, IgE, neuropolypeptides, insulin, vaccines, growth factors for example but not limited to epidermal growth factor, keratinocyte growth factor, transformation growth factor, growth regulators, fragments thereof, or combinations thereof. Non-limiting example of a protein of interest to be expressed include therapeutic protein, viral proteins, antibody or vaccine component. In the examples provided below rituximab is used as a non-limiting example of protein of interest. Similar results described herein are observed with other glycoproteins, for example IgG, and HA, and it is to be understood that other proteins of interest may be used according to the methods described herein.

Furthermore, the present invention pertains to a plant, a plant cell, or a seed, comprising a nucleotide sequence encoding RMD operatively linked with a regulatory region that is active in the plant. The plant, plant cell, or seed may further comprise a second nucleotide sequence encoding one or more than one of a protein of interest, the second nucleotide sequence operatively linked to one or more than one second regulatory region active within the plant. The first nucleotide sequence, the second nucleotide sequence, or both the first nucleotide sequence and the second nucleotide sequence, may be codon optimized for expression within the plant, plant cell or plant seed.

By the term “portion of a plant”, it is meant any part derived from a plant, including the entire plant, tissue obtained from the plant for example but not limited to the leaves, the leaves and stem, the roots, the aerial portion including the leaves, stem and optionally the floral portion of the plant, cells or protoplasts obtained from the plant.

By the term “plant matter”, it is meant any material derived from a plant. Plant matter may comprise an entire plant, tissue, cells, or any fraction thereof. Further, plant matter may comprise intracellular plant components, extracellular plant components, liquid or solid extracts of plants, or a combination thereof. Further, plant matter may comprise plants, plant cells, tissue, a liquid extract, or a combination thereof, from plant leaves, stems, fruit, roots or a combination thereof. Plant matter may comprise a plant or portion thereof which has not been subjected to any processing steps. However, it is also contemplated that the plant material may be subjected to minimal processing steps as defined below, or more rigorous processing, including partial or substantial protein purification using techniques commonly known within the art including, but not limited to chromatography, electrophoresis and the like.

By the term “minimal processing” it is meant plant matter, for example, a plant or portion thereof comprising a protein of interest which is partially purified to yield a plant extract, homogenate, fraction of plant homogenate or the like. Partial purification may comprise, but is not limited to disrupting plant cellular structures thereby creating a composition comprising soluble plant components, and insoluble plant components which may be separated for example, but not limited to, by centrifugation, filtration or a combination thereof. In this regard, proteins secreted within the extracellular space of leaf or other tissues could be readily obtained using vacuum or centrifugal extraction, or tissues could be extracted under pressure by passage through rollers or grinding or the like to squeeze or liberate the protein free from within the extracellular space. Minimal processing could also involve preparation of crude extracts of soluble proteins, since these preparations would have negligible contamination from secondary plant products. Further, minimal processing may involve aqueous extraction of soluble protein from leaves, followed by precipitation with any suitable salt. Other methods may include large scale maceration and juice extraction in order to permit the direct use of the extract.

The plant matter, in the form of plant material or tissue may be orally delivered to a subject. The plant matter may be administered as part of a dietary supplement, along with other foods, or encapsulated. The plant matter or tissue may also be concentrated to improve or increase palatability, or provided along with other materials, ingredients, or pharmaceutical excipients, as required.

It is contemplated that a plant comprising the protein of interest may be administered to a subject, for example an animal or human, in a variety of ways depending upon the need and the situation. For example, the protein of interest obtained from the plant may be extracted prior to its use in either a crude, partially purified, or purified form. If the protein is to be purified, then it may be produced in either edible or non-edible plants. Furthermore, if the protein is orally administered, the plant tissue may be harvested and directly feed to the subject, or the harvested tissue may be dried prior to feeding, or an animal may be permitted to graze on the plant with no prior harvest taking place. It is also considered within the scope of this invention for the harvested plant tissues to be provided as a food supplement within animal feed. If the plant tissue is being feed to an animal with little or no further processing it is preferred that the plant tissue being administered is edible.

By “operatively linked” it is meant that the particular sequences interact either directly or indirectly to carry out an intended function, such as mediation or modulation of gene expression. The interaction of operatively linked sequences may, for example, be mediated by proteins that interact with the operatively linked sequences. A transcriptional regulatory region and a sequence of interest are operably linked when the sequences are functionally connected so as to permit transcription of the sequence of interest to be mediated or modulated by the transcriptional regulatory region.

The RMD protein, protein of interest, the hybrid protein or a combination thereof maybe expressed in an expression system that comprises amplification elements and/or regulatory elements or regions (also referred to herein as enhancer elements).

For example an amplification element from a geminivirus such as for example, an amplification element from the bean yellow dwarf virus (BeYDV) may be used to express the RMD protein, protein of interest or the hybrid protein. BeYDV belongs to the Mastreviruses genus adapted to dicotyledonous plants. BeYDV is monopartite having a single-strand circular DNA genome and can replicate to very high copy numbers by a rolling circle mechanism. BeYDV-derived DNA replicon vector systems have been used for rapid high-yield protein production in plants.

Furthermore enhancer elements may be used to achieve high level of transient expression of RMD, the protein of interest or the hybrid protein. Enhancer elements may be based on RNA plant viruses, including comoviruses, such as Cowpea mosaic virus (CPMV; see, for example, WO2007/135480; WO2009/087391; US 2010/0287670, Sainsbury F. et al., 2008, Plant Physiology; 148: 121-1218; Sainsbury F. et al., 2008, Plant Biotechnology Journal; 6: 82-92; Sainsbury F. et al., 2009, Plant Biotechnology Journal, 7: 682-693; Sainsbury F. et al. 2009, Methods in Molecular Biology, Recombinant Proteins From Plants, vol. 483: 25-3.9).

Other known expression enhancers may also be used, for example, expression enhancers obtained from plant sequences including, but not limited to, AtPsaK (Arabidopsis thaliana psaK), AtPsaK 5′, AtPsaK 3′, NbPsaK1 (Nicotiana benthamiana psaK), NbPsaK1 3′, NbPsaK2, NbPsaK2 3′, as described in Diamos et al. (2016, Frontiers in Plant Science 7:1-15, which is incorporated herein by reference)

In one embodiment the Enhancer Elements may be “CPMVX” (also referred as “CPMV 160”) and/or “CPMVX+” (also referred to as “CPMV 160+”) as described in U.S. 61/925,852, PCT/CA2015/050009 and PCT/CA2015/050240 which are incorporated herein by reference.

Expression enhancer “CPMVX” comprises a comovirus cowpea mosaic virus (CPMV) 5′ untranslated region (UTR). The 5′UTR from nucleotides 1-160 of the CPMV RNA-2 sequence (SEQ ID NO: 1), starts at the transcription start site to the first in frame initiation start codon (at position 161), which serve as the initiation site for the production of the longer of two carboxy coterminal proteins encoded by a wild-type comovirus genome segment. Furthermore a ‘third’ initiation site at (or corresponding to) position 115 in the CPMV RNA-2 genomic sequence may also be mutated, deleted or otherwise altered. It has been shown that removal of AUG 115 in addition to the removal of AUG 161 enhances expression when combined with an incomplete M protein (Sainsbury and Lomonossoff, 2008, Plant Physiology; 148: 1212-1218; WO 2009/087391; which are incorporated herein by reference).

CPMVX comprises X nucleotides of SEQ ID NO: 1, where X=160, 155, 150, or 114 of SEQ ID NO: 1, or a sequence that comprises between 80% to 100% sequence similarity with CPMVX, where X=160, 155, 150, or 114 of SEQ ID NO: 1. This expression enhancer is generally referred to as CPMVX.

The expression enhancer CPMVX, where X=160, consists of nucleotides 1-160 of SEQ ID NO: 1:

(SEQ ID NO: 1)

| | |
|---|---|
| 1 | tattaaaatc ttaataggtt ttgataaaag cgaacgtggg |
| | gaaacccgaa ccaaaccttc |
| 61 | ttctaaactc tctctcatct ctcttaaagc aaacttctct |
| | cttgtctttc ttgcgtgagc |
| 121 | gatcttcaac gttgtcagat cgtgcttcgg caccagtaca |

The CPMVX enhancer sequence may further be fused to a stuffer sequence, wherein the CMPVX comprises X nucleotides of SEQ ID NO: 1, where X=160, 155, 150, or 114 of SEQ ID NO: 1, or a sequence that comprises between 80 to 100% sequence similarity with CPMVX, where X=160, 155, 150, or 114 of SEQ ID NO: 1, and the stuffer sequence comprises from 1-100 nucleotides fused to the 3′ end of the CMPVX sequence. For example, the stuffer sequence may comprise from about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 nucleotides, or any number of nucleotides therebetween.

If the CMPVX sequence comprises a stuffer fragment, then this expression enhancer may be referred to as CPMVX+, where X=160, 155, 150, 114 of SEQ ID NO: 1, it may also be referred to as CMPVX comprising a stuffer sequence, or it may be referred to as CPMV160+; CPMV155+; CPMV150+; CPMV114+, when X-160, 155, 150, or 114, respectively. Constructs comprising CPMVX that do not comprise a stuffer sequence may be termed CPMVX+, where X=160, 155, 150, 114 of SEQ ID NO: 1, and where the stuffer sequence is of 0 nucleotides in length.

The stuffer sequence may be modified by truncation, deletion, or replacement of the native CMPV5′UTR sequence that is located 3′ to nucleotide 160. The modified stuffer sequence may be removed, replaced, truncated or shortened when compared to the initial or unmodified (i.e. native) stuffer sequence associated with the 5′UTR (as described in Sainsbury F., and Lomonossoff G. P., 2008, Plant Physiol. 148: pp. 1212-1218). The stuffer sequence may comprise a one or more restriction sites (polylinker, multiple cloning site, one or more cloning sites), one or more plant kozak sequences, one or more linker sequences, one or more recombination sites, or a combination thereof. For example, which is not to be considered limiting, a stuffer sequence may comprise in series, a multiple cloning site of a desired length fused to a plant kozak sequence. The stuffer sequence does not comprise a nucleotide sequence from the native 5′UTR sequence that is positioned 3′ to nucleotide 160 of the native CPMV 5′UTR, for example nucleotides 161 to 512 as shown in FIG. 1 of Sainsbury F., and Lomonossoff G. P. (2008, Plant Physiol. 148: pp. 1212-1218; which is incorporated herein by reference). That is, the incomplete M protein present in the prior art CPMV HT sequence (FIG. 1; of Sainsbury F., and Lomonossoff G. P., 2008) is removed from the 5′UTR in the present invention.

Plant Kozak consensus sequences are known in the art (see for example Rangan et al. Mol. Biotechnol., 2008, July 39(3), pp. 207-213). Both naturally occurring and synthetic Kozak sequences may be used in the expression enhancer or may be fused to the nucleotide sequence of interest as described herein.

The plant kozak sequence may be any known plant kozak sequences (see for example L. Rangan et. al. Mol. Biotechnol. 2008), including, but not limited to the following plant consensus sequences:

caA(A/C)a (SEQ ID NO: 2; plant kingdom)
aaA(A/C)a (SEQ ID NO: 3; dicots)
aa(A/G)(A/C)a (SEQ ID NO: 4; arabidopsis)

The plant kozak sequence may also be selected from the group of:

AGAAA (SEQ ID NO: 5)
AGACA (SEQ ID NO: 6)
AGGAA (SEQ ID NO: 7)
AAAAA (SEQ ID NO: 8)
AAACA (SEQ ID NO: 9)
AAGCA (SEQ ID NO: 10)
AAGAA (SEQ ID NO: 11)
AAAGAA (SEQ ID NO: 12)
AAAGAA (SEQ ID NO: 13)
(A/-)A(A/G) (A/G) (A/C)A. (SEQ ID NO: 14;
Consensus sequence)

The expression enhancer CPMVX, or CPMVX+, may be operatively linked at the 5′end of the enhancer sequence with a regulatory region that is active in a plant, and operatively linked to a nucleotide sequence of interest at the 3′end of the expression enhancer, in order to drive expression of the nucleotide sequence of interest within a plant host.

### CPMV HT+, CPMV HT+[WT115], CPMV HT+[511]

In another embodiment the Enhancer Elements is “CPMV HT+” as described in U.S. 61/971,274, PCT/CA2015/050009 and PCT/CA2015/050240 which are incorporated herein by reference. Expression enhancer “CPMV HT+” comprises a comovirus 5′ untranslated region (UTR) and a modified, lengthened, or truncated stuffer sequence.

A plant expression system comprising a first nucleic acid sequence comprising a regulatory region, operatively linked with one or more than one expression enhancer as described herein (e.g. CPMV HT+, CPMV HT+[WT115], CPMV HT+[511]), and a nucleotide sequence encoding a RMD, a protein of interest or hybrid protein is also provided. Furthermore, a nucleic acid comprising a promoter (regulatory region) sequence, an expression enhancer (e.g. CPMV HT+ or CPMV HT+[WT 115]) comprising a comovirus 5′UTR and a stuffer sequence with a plant kozak sequence fused to one or more nucleic acid sequences encoding a RMD, the protein of interest or hybrid protein are described. The nucleic acid may further comprise a sequence comprising a comovirus 3′ untranslated region (UTR), for example, a plastocyanin 3′ UTR, or other 3′UTR active in a plant, and a terminator sequence, for example a NOS terminator, operatively linked to the 3′end of the nucleotide sequence encoding RMD, the protein of interest or hybrid protein, so that the nucleotide sequence encoding RMD, the protein of interest or hybrid protein is inserted upstream from the comovirus 3′ untranslated region (UTR), plastocyanin 3′ UTR, or other 3′UTR sequence.

SEQ ID NO:15 comprises a “CPMV HT” expression enhancer as known in the prior art (e.g. FIG. 1 of Sainsbury and Lomonossoff 2008, Plant Physiol. 148: pp. 1212-1218; which is incorporated herein by reference). CPMV HT includes the 5′UTR sequence from nucleotides 1-160 of SEQ ID NO:15 with modified nucleotides at position 115 (cgt), and an incomplete M protein with a modified nucleotide at position 162 (acg), and lacks a plant kozak sequence (5′UTR: nucleotides 1-160; incomplete M protein underlined, nucleotides 161-509). SEQ ID NO:15 also includes a multiple cloning site (italics, nucleotides 510-528) which is not present in the prior art CPMV HT sequence:

SEQ ID NO: 15

| | |
|---|---|
| 1 | tattaaaatc ttaataggtt ttgataaaag cgaacgtggg |
| | gaaacccgaa ccaaaccttc |
| 61 | ttctaaactc tctctcatct ctcttaaagc aaacttctct |
| | cttgtctttc ttgcgtgagc |
| 121 | gatcttcaac gttgtcagat cgtgcttcgg caccagtaca |
| | [symbol] ttttctt tcactgaagc |
| 181 | gaaatcaaag atctctttgt ggacacgtag tgcggcgcca |
| | ttaaataacg tgtacttgtc |
| 241 | ctattcttgt cggtgtggtc ttgggaaaag aaagcttgct |
| | ggaggctgct gttcagcccc |
| 301 | atacattact tgttacgatt ctgctgactt tcggcgggtg |
| | caatatctct acttctgctt |
| 361 | gacgaggtat tgttgcctgt acttctttct tcttcttctt |
| | gctgattggt tctataagaa |
| 421 | atctagtatt ttctttgaaa cagagttttc ccgtggtttt |
| | cgaacttgga gaaagattgt |
| 481 | taagcttctg tatattctgc ccaaatttgt cgggccc |

CPMV HT+ with a plant kozak consensus sequence is provided in SEQ ID NO: 16 (nucleotide 1-160, 5′UTR, including modified ATG at positions 115 (GTG) lower case bold and italics; stuffer fragment comprising: an incomplete M protein underlined, nucleotides 161-509, with modified nucleotide at 162 (ACG); a multiple cloning site, italics, nucleotides 510-528; and a consensus plant kozak sequence, caps and bold, nucleotides 529-534).

(SEQ ID NO: 16)

| | |
|---|---|
| 1 | tattaaaatc ttaataggtt ttgataaaag cgaacgtggg |
| | gaaacccgaa ccaaaccttc |
| 61 | ttctaaactc tctctcatct ctcttaaagc aaacttctct |
| | cttgtctttc ttgc [symbol] agc |
| 121 | gatcttcaac gttgtcagat cgtgcttcgg caccagtaca |
| | [symbol] ttttctt tcactgaagc |
| 181 | gaaatcaaag atctctttgt ggacacgtag tgcggcgcca |
| | ttaaataacg tgtacttgtc |
| 241 | ctattcttgt cggtgtggtc ttgggaaaag aaagcttgct |
| | ggaggctgct gttcagcccc |
| 301 | atacattact tgttacgatt ctgctgactt tcggcgggtg |
| | caatatctct acttctgctt |
| 361 | gacgaggtat tgttgcctgt acttctttct tcttcttctt |
| | gctgattggt tctataagaa |
| 421 | atctagtatt ttctttgaaa cagagttttc ccgtggtttt |
| | cgaacttgga gaaagattgt |
| 481 | taagcttctg tatattctgc ccaaatttgt tcgggcccaa |
| | taccgcgg [symbol] [symbol] |

SEQ ID NO: 17 (“CPMV HT+511”) comprises a segment of the native sequence of the CPMV RNA 2 genome from nucleotides 1-154. The 5′UTR sequence from nucleotides 1-511 of SEQ ID NO:17 comprises modified “atg” sequences at positions 115 (“g” in place of “a”; italics bold) and 162 (“c” in place of “t”; italics bold), and an incomplete M protein (underlined) from nucleotides 161-511. CPMV HT+511 comprises a native M protein kozak consensus sequence (nucleotides 508-511; bold):

SEQ ID NO: 17

| | |
|---|---|
| 1 | tattaaaatc ttaataggtt ttgataaaag cgaacgtggg |
| | gaaacccgaa ccaaaccttc |
| 61 | ttctaaactc tctctcatct ctcttaaagc aaacttctct |
| | cttgtctttc ttgc [symbol] agc |
| 121 | gatcttcaac gttgtcagat cgtgcttcgg caccagtaca |
| | [symbol] ttttctt tcactgaagc |
| 181 | gaaatcaaag atctctttgt ggacacgtag tgcggcgcca |
| | ttaaataacg tgtacttgtc |
| 241 | ctattcttgt cggtgtggtc ttgggaaaag aaagcttgct |
| | ggaggctgct gttcagcccc |
| 301 | atacattact tgttacgatt ctgctgactt tcggcgggtg |
| | caatatctct acttctgctt |
| 361 | gacgaggtat tgttgcctgt acttctttct tcttcttctt |
| | gctgattggt tctataagaa |
| 421 | atctagtatt ttctttgaaa cagagttttc ccgtggtttt |
| | cgaacttgga gaaagattgt |
| 481 | taagcttctg tatattctgc ccaaatttga a . . . |

Another non-limiting example of a CPMV HT+enhancer sequence is provided by the sequence of SEQ ID NO: 18 (CPMV HT+[WT115]). Expression cassettes or vectors comprising CPMV HT+ and including a plant regulatory region in operative association with the expression enhancer sequence of SEQ ID NO: 18, and the transcriptional start site (ATG) at the 3′ end fused to a nucleotide sequence encoding RMD, the protein of interest or hybrid protein are also part of the present invention.

SEQ ID NO: 18 (CPMV HT+[WT115]) nucleotide 1-160, 5′UTR, with an ATG at position 115-117, lower case bold; stuffer fragment comprising: an incomplete M protein underlined, nucleotides 161-509; with a modified ATG at position 161-153 lower case bold, and underlined, a multiple cloning site, italics, nucleotides 510-528; and a plant kozak sequence, caps and bold, nucleotides 529-534).

(SEQ ID NO: 18)

| | |
|---|---|
| 1 | tattaaaatc ttaataggtt ttgataaaag cgaacgtggg |
| | gaaacccgaa ccaaaccttc |
| 61 | ttctaaactc tctctcatct ctcttaaagc aaacttctct |
| | cttgtctttc ttgc [symbol] agc |
| 121 | gatcttcaac gttgtcagat cgtgcttcgg caccagtaca |
| | [symbol] ttttctt tcactgaagc |
| 181 | gaaatcaaag atctctttgt ggacacgtag tgcggcgcca |
| | ttaaataacg tgtacttgtc |
| 241 | ctattcttgt cggtgtggtc ttgggaaaag aaagcttgct |
| | ggaggctgct gttcagcccc |
| 301 | atacattact tgttacgatt ctgctgactt tcggcgggtg |
| | caatatctct acttctgctt |
| 361 | gacgaggtat tgttgcctgt acttctttct tcttcttctt |
| | gctgattggt tctataagaa |
| 421 | atctagtatt ttctttgaaa cagagttttc ccgtggtttt |
| | cgaacttgga gaaagattgt |
| 481 | taagcttctg tatattctgc ccaaatttgt tcgggcccaa |
| | taccgcgg [symbol] |

The plant kozak sequence of SEQ ID NO: 18 may be any plant kozak sequence, including but not limited, to one of the sequences of SEQ ID NO's: 2-14.

The one or more than one nucleotide sequence of the present invention may be expressed in any suitable plant host that is transformed by the nucleotide sequence, or constructs, or vectors of the present invention. Examples of suitable hosts include, but are not limited to, agricultural crops including alfalfa, canola, Brassica spp., maize, Nicotiana spp., alfalfa, potato, ginseng, pea, oat, rice, soybean, wheat, barley, sunflower, and cotton.

TABLE 1
Examples of constructs that have been prepared
as described herein:

| | Expression | | |
|---|---|---|---|
| Constr. # | Enhancer | Description | FIG. |
| 5072 | CPMV 160+ | PDISP-LC and PDISP-HC of rituximab | 14B |
| | | mAb | |
| 5091 | CPMV 160+ | Flag-RMD | 8I |
| 5092 | CPMV 160 | Flag-RMD | 9E |
| 5093 | CPMV 160+ | paRMD | 10D |
| 5094 | CPMV 160 | paRMD | 11C |
| 3431 | CMPV 160 | atRMD | 15F |
| 3432 | CMPV 160 | pbRMD | 16F |
| 3433 | CMPV 160 | psRMD | 17F |
| 3434 | CMPV 160 | xvRMD | 18F |

TABLE 2
Description of sequences

| SEQ ID | | |
|---|---|---|
| NO: | Description | FIG. |
| 1 | CPMVX | — |
| 2 | Kozak consensus | — |
| | sequence plant | |
| | kingdom | |
| 3 | Kozak consensus | — |
| | sequence dicots | |
| 4 | Kozak consensus | — |
| | sequence arabidopsis | |
| 5-13 | Plant kozak sequences | — |
| 14 | Kozak consensus | — |
| | sequence | |
| 15 | CPMV HT | — |
| 16 | CPMV HT+ | — |
| 17 | CPMV HT+ 511 | — |
| 18 | CPMV HT+ [WT115] | — |
| 19 | Primer Flag_RMD Fw | 8A |
| 20 | Primer | 8B |
| | 5091_5092_IF_REV | |
| 21 | Pseudomonas | 8C |
| | aeruginosa RMD | |
| | (optimized) | |
| 22 | Primer 5091_IF_FW | 8D |
| 23 | Construct 2171 | 8F |
| 24 | Construct 5091 | 8G |
| 25 | AA sequence of | 8H |
| | FLAG-Nter-RMD from | |
| | Pseudomonas | |
| | aeruginosa | |
| 26 | Primer 5092_IF_Fw | 9A |
| 27 | Construct 1190 | 9C |
| 28 | Construct 5092 | 9D |
| 29 | Primer 5093_IF_Fw | 10A |
| 30 | Construct 5093 | 10B |
| 31 | Amino acid sequence | 10C |
| | of RMD from | |
| | Pseudomonas | |
| | aeruginosa | |
| 32 | Primer 5094_IF_Fw | 11A |
| 33 | Construct 5094 | 11B |
| 34 | Primer IF**(SacII)- | 12A |
| | PDI.s1+4c | |
| 35 | Primer IF**- | 12B |
| | HC(Ritux).s1-6r | |
| 36 | PDISP/HC rituximab | 12C |
| 37 | Construct 2109 | 12D |
| 38 | AA sequence of | 12E |
| | PDISP/HC rituximab | |
| | mAb | |
| 39 | Primer IF**- | 13A |
| | LC(Ritux).s1-6r | |
| 40 | Coding sequence of | 13B |
| | PDISP/HC rituximab | |
| 41 | Construct 2129 | 13C |
| 42 | AA sequence of | 13D |
| | PDISP/LC rituximab | |
| | mAb | |
| 43 | Construct 5072 | 14A |
| 44 | Primer IF-atRMD(opt).c | 15A |
| 45 | Primer IF-atRMD(opt).r | 15B |
| 46 | Agrobacterium | 15C |
| | tumefaciens RMD | |
| | (strain TS43) | |
| | optimized | |
| 47 | Construct 3431 (NA) | 15D |
| 48 | AA sequence of | 15E |
| | Agrobacterium | |
| | tumefaciens RMD | |
| | (strain TS43) | |
| 49 | Primer IF- | 16A |
| | pbRMD(opt).c | |
| 50 | Primer IF- | 16B |
| | pbRMD(opt).r | |
| 51 | Pseudomonas | 16C |
| | brassicacearum RMD | |
| | (strain NFM421) | |
| | optimized | |
| 52 | Construct 3432 | 16D |
| 53 | AA sequence of | 16E |
| | Pseudomonas | |
| | brassicacearum RMD | |
| | (strain NFM421) | |
| 54 | Primer IF- | 17A |
| | psRMD(opt).c | |
| 55 | Primer IF- | 17B |
| | psRMD(opt).r | |
| 56 | Pseudomonas | 17C |
| | syringae RMD | |
| | optimized | |
| 57 | Construct 3433 | 17D |
| 58 | AA sequence of | 17E |
| | Pseudomonas | |
| | syringae RMD | |
| 59 | Primer IF- | 18A |
| | xvRMD(opt).c | |
| 60 | Primer IF- | 18B |
| | xvRMD(opt).r | |
| 61 | Xanthomonas | 18C |
| | vasicola RMD (strain | |
| | NCPPB1326) | |
| | optimized | |
| 62 | Construct 3433 | 18D |
| 63 | AA of sequence of | 18E |
| | Xanthomonas | |
| | vasicola RMD (strain | |
| | NCPPB1326) | |

The present invention will be further illustrated in the following examples.

### EXAMPLES

### Material and Methods: Assembly of Expression Cassettes:

G-2X35S/CPMV 160+/PDISP/HC Rituximab/NOS+2X35S/CPMV 160+/PDISP/LC rituximab/NOS (Construct number 5072; also termed “Ritux”)

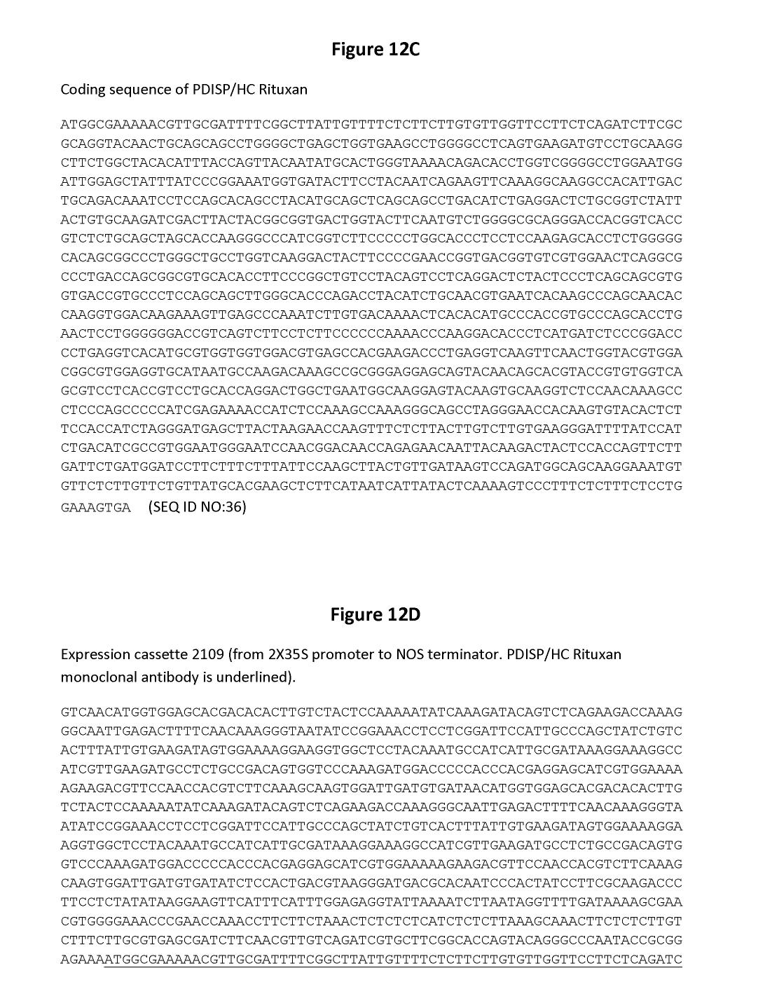

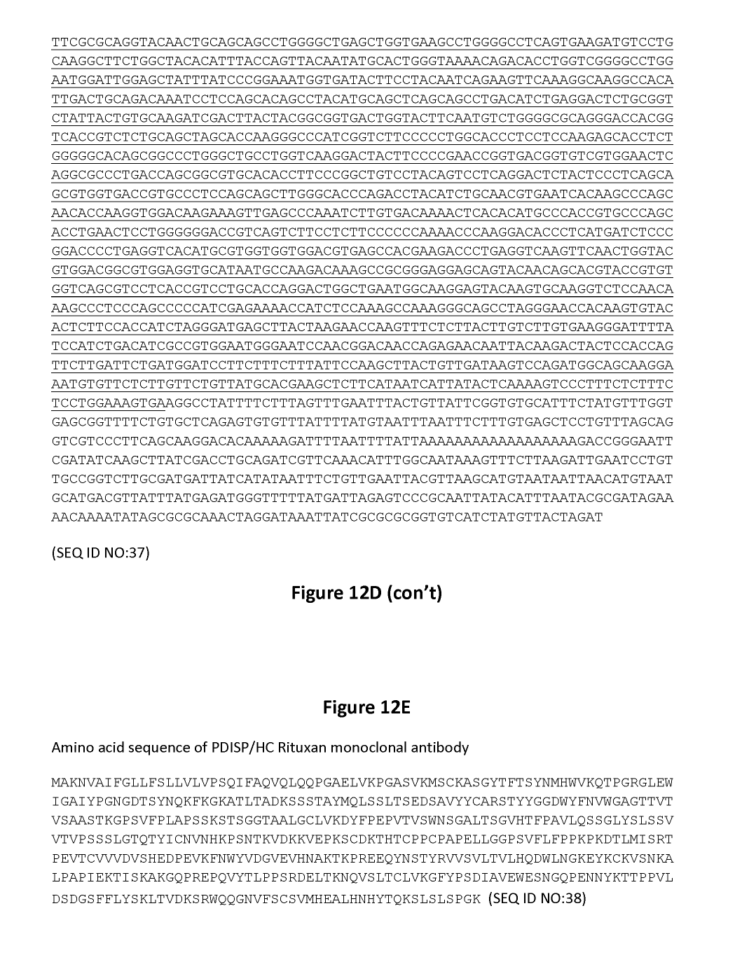

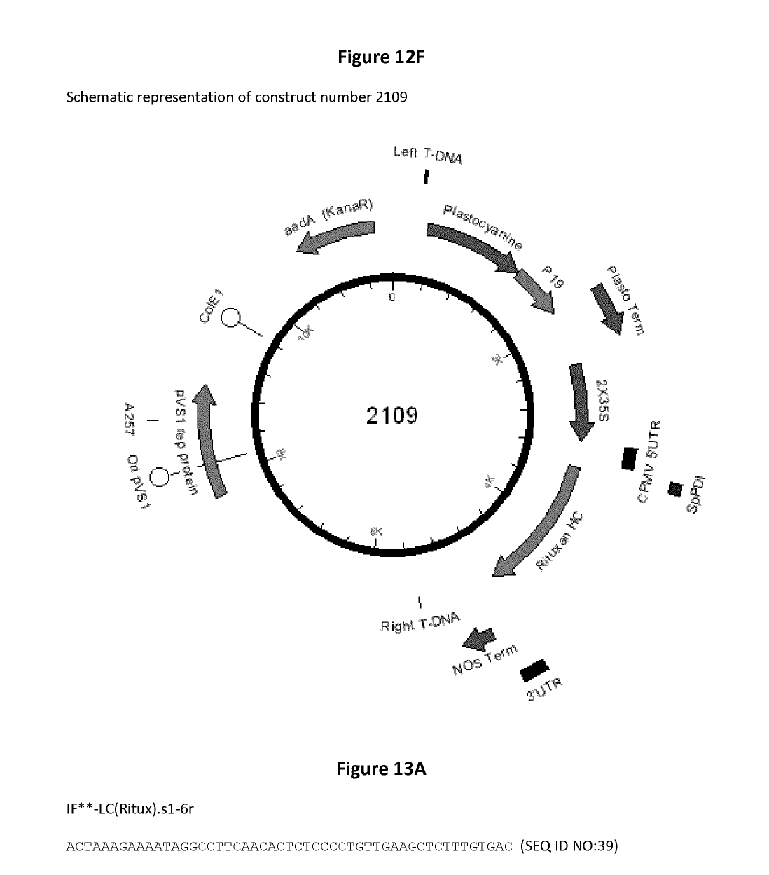

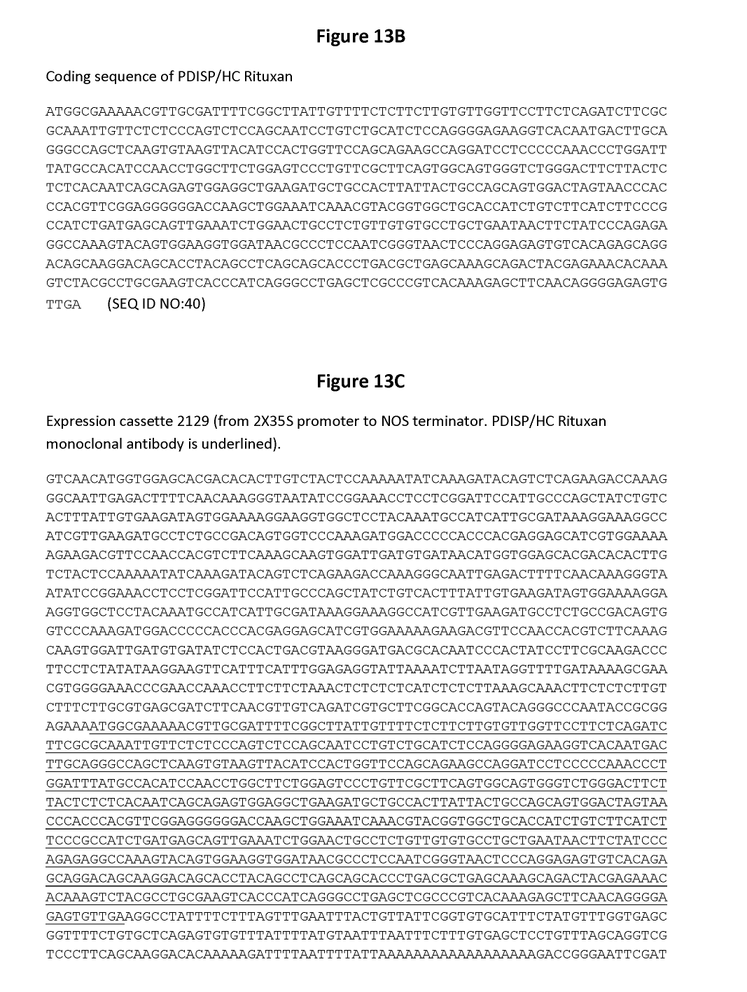

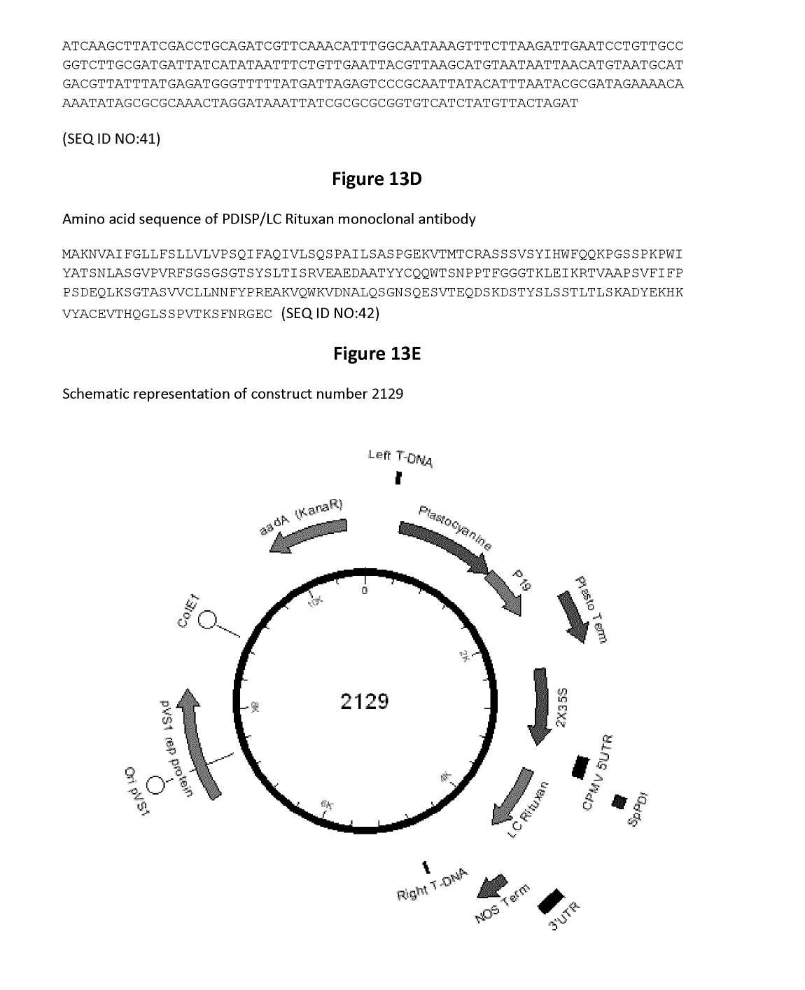

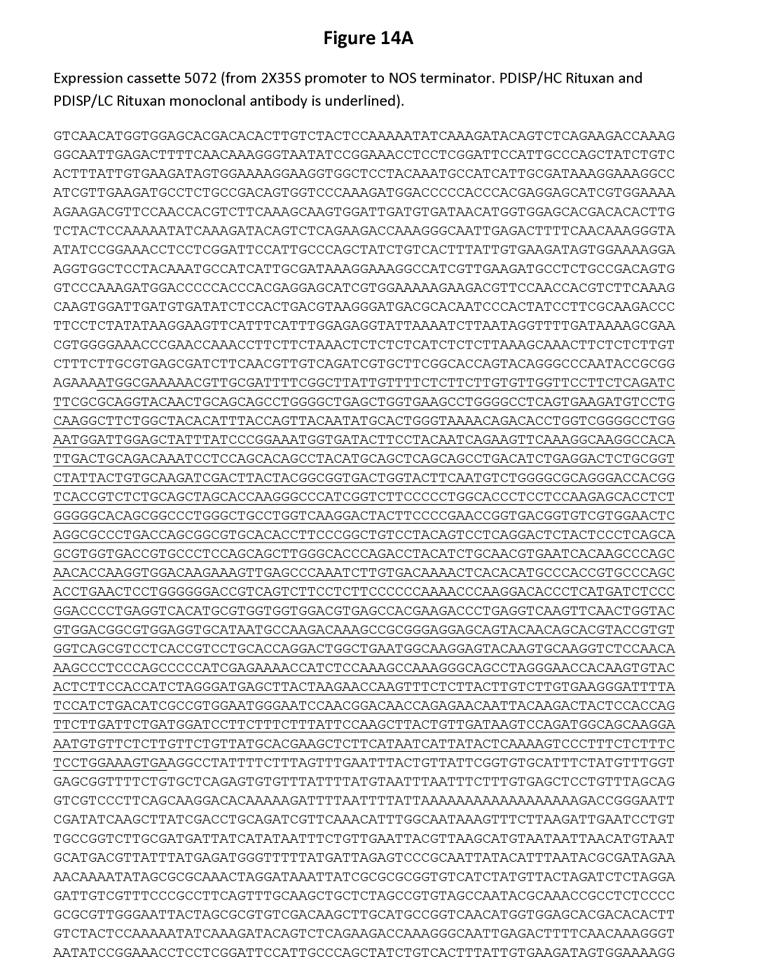

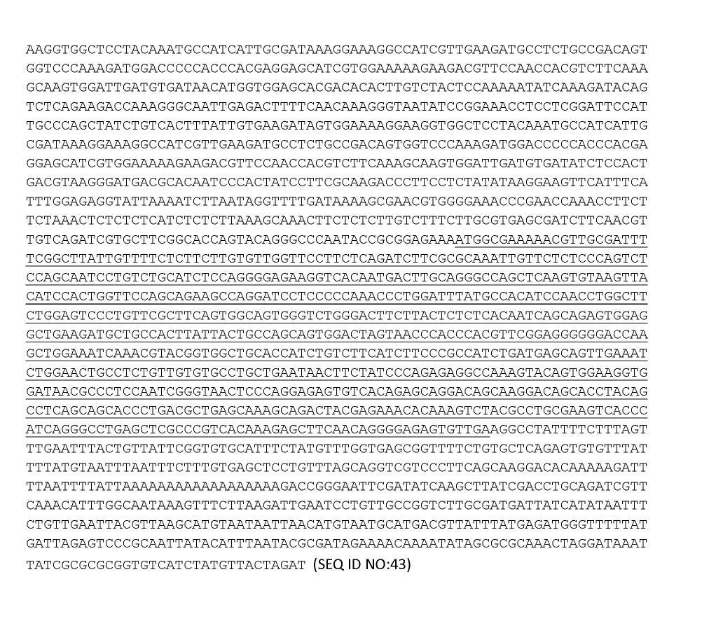

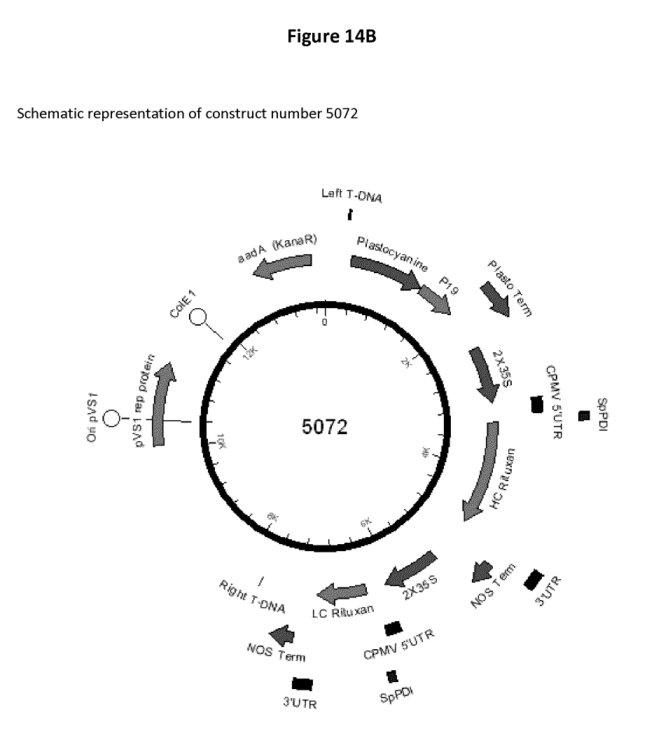

A plasmid allowing the dual expression of light chain and heavy chain from rituximab monoclonal antibody was assembled as follow. Construct number 2129 (see below; FIG. 13C, SEQ ID:41) was digested with AvrII and AscI restriction enzyme. The resulting fragments, comprising the complete cassette for the expression of PDISP/LC rituximab, was inserted into construct number 2109 (see below; FIG. 12D, SEQ ID: 37), comprising the complete expression cassette for the expression of PDISP HC/rituximab, previously digested with XbaI and AscI restriction enzyme, by ligation. The resulting construct was given number 5072 (FIG. 14A, SEQ ID NO: 43). The amino acid sequence of PDISP/LC rituximab monoclonal antibody is presented in FIG. 13D (SEQ ID NO:42) and the amino acid sequence of PDISP/HC rituximab monoclonal antibody is presented in FIG. 12E (SEQ ID NO:38). A representation of plasmid 5072 is presented in FIG. 14B.

2X35S/CPMV 160+/PDISP/LC Rituximab/NOS (Construct number 2129)

C2B8 (rituximab) is a chimeric (mouse/human) monoclonal antibody directed against the B-cell-specific antigen CD20 expressed on non-Hodgkin's lymphomas (NHL). Rituximab mediates complement and antibody-dependent cell-mediated cytotoxicity and has direct antiproliferative effects against malignant B-cell lines in vitro (N Selenko et. al., Leukemia, October 2001, 15 (10); 1619-1626). A sequence encoding light chain from rituximab monoclonal antibody in which the native signal peptide has been replaced by that of alfalfa protein disulfide isomerase (PDISP/LC rituximab) was cloned into 2X35S/CPMV 160+/NOS expression system using the following PCR-based method. A fragment containing the PDISP/LC rituximab coding sequence was amplified using primers IF**(SacII)-PDI.s1+4c (FIG. 12A, SEQ ID NO:34) and IF**-LC(Ritux).s1-6r (FIG. 13A, SEQ ID NO:39), using PDISP/LC rituximab gene sequence (FIG. 13B, SEQ ID NO:40) as template. The PCR product was cloned in 2X35S/CPMV 160+/NOS expression system using In-Fusion cloning system (Clontech, Mountain View, Calif.). Construct number 2171 (FIG. 8E) was digested with AatII and StuI restriction enzyme and the linearized plasmid was used for the In-Fusion assembly reaction. Construct number 2171 is an acceptor plasmid intended for “In Fusion” cloning of genes of interest in a 2X35S/CPMV 160+/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 8F (SEQ ID NO:23). The resulting construct was given number 2129 (FIG. 13C, SEQ ID NO:41). The amino acid sequence of PDISP/LC rituximab monoclonal antibody is presented in FIG. 13D (SEQ ID NO:42). A representation of plasmid 2109 is presented in FIG. 13E.

2X35 S/CPMV 160+/PDISP/HC Rituximab/NOS (Construct number 2109)

C2B8 (rituximab) is a chimeric (mouse/human) monoclonal antibody directed against the B-cell-specific antigen CD20 expressed on non-Hodgkin's lymphomas (NHL). Rituximab mediates complement and antibody-dependent cell-mediated cytotoxicity and has direct antiproliferative effects against malignant B-cell lines in vitro (N Selenko et. al., Leukemia, October 2001, 15 (10); 1619-1626). A sequence encoding heavy chain from rituximab monoclonal antibody in which the native signal peptide has been replaced by that of alfalfa protein disulfide isomerase (PDISP/HC rituximab) was cloned into 2X35S/CPMV 160+/NOS expression system using the following PCR-based method. A fragment containing the PDISP/HC rituximab coding sequence was amplified using primers IF**(SacII)-PDI.s1+4c (FIG. 12A, SEQ ID NO:34) and IF**-HC(Ritux).s1-6r (FIG. 12B, SEQ ID NO:35), using PDISP/HC rituximab gene sequence (FIG. 12C, SEQ ID NO:36) as template. The PCR product was cloned in 2X35S/CPMV 160+/NOS expression system using In-Fusion cloning system (Clontech, Mountain View, Calif.). Construct number 2171 (FIG. 8E) was digested with AatII and StuI restriction enzyme and the linearized plasmid was used for the In-Fusion assembly reaction. Construct number 2171 is an acceptor plasmid intended for “In Fusion” cloning of genes of interest in a 2X35S/CPMV 160+/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 8F (SEQ ID NO:23). The resulting construct was given number 2109 (Figure E412D, SEQ ID NO:37). The amino acid sequence of PDISP/HC rituximab monoclonal antibody is presented in FIG. 12E (SEQ ID NO:38). A representation of plasmid 2109 is presented in FIG. 12F.

2X35S/CPMV 160+/Flag-Nter-RMD(opt)/NOS (Construct number 5091; also termed: “160+/Flag-RMD”)

An optimized sequence encoding RMD from Pseudomonas aeruginosa strain PAO1 tagged in N-terminal with a FLAG in frame was cloned into 2X35 S/CPMV 160+/NOS expression system using the following PCR-based method. A fragment containing the RMD coding sequence was first amplified to add the FLAG tag in N-Ter using primers Flag_Rmd_Fw (FIG. 8A, SEQ ID NO: 19) and 5091_5092_IF_Rev (FIG. 8B, SEQ ID NO:20), using optimized RMD gene sequence (FIG. 8C, SEQ ID NO:21) as template. For sequence optimization, RMD protein sequence (Genbank accession number AAG08839.1) was backtranslated and optimized for human codon usage, GC content and mRNA structure. The PCR product was used as template for a second amplification using 5091 IF_Fw (FIG. 8D, SEQ ID NO:22) and 5091_5092_IF_Rev (FIG. 8B, SEQ ID NO:20). The final PCR product was cloned in 2X35S/CPMV 160+/NOS expression system using In-Fusion cloning system (Clontech, Mountain View, Calif.). Construct number 2171 (FIG. 8E) was digested with SacII and StuI restriction enzyme and the linearized plasmid was used for the In-Fusion assembly reaction. Construct number 2171 is an acceptor plasmid intended for “In Fusion” cloning of genes of interest in a 2X35 S/CPMV 160+/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 8F (SEQ ID NO:23). The resulting construct was given number 5091 (FIG. 8G, SEQ ID NO:24). The amino acid sequence of FLAG-Nter-RMD from Pseudomonas aeruginosa strain PAO1 is presented in FIG. 8H (SEQ ID NO:25). A representation of plasmid 5091 is presented in FIG. 8I.

2X35S/CPMV 160/Flag-Nter-RMD(opt)/NOS (Construct number 5092; also termed: “160/Flag-RMD”)

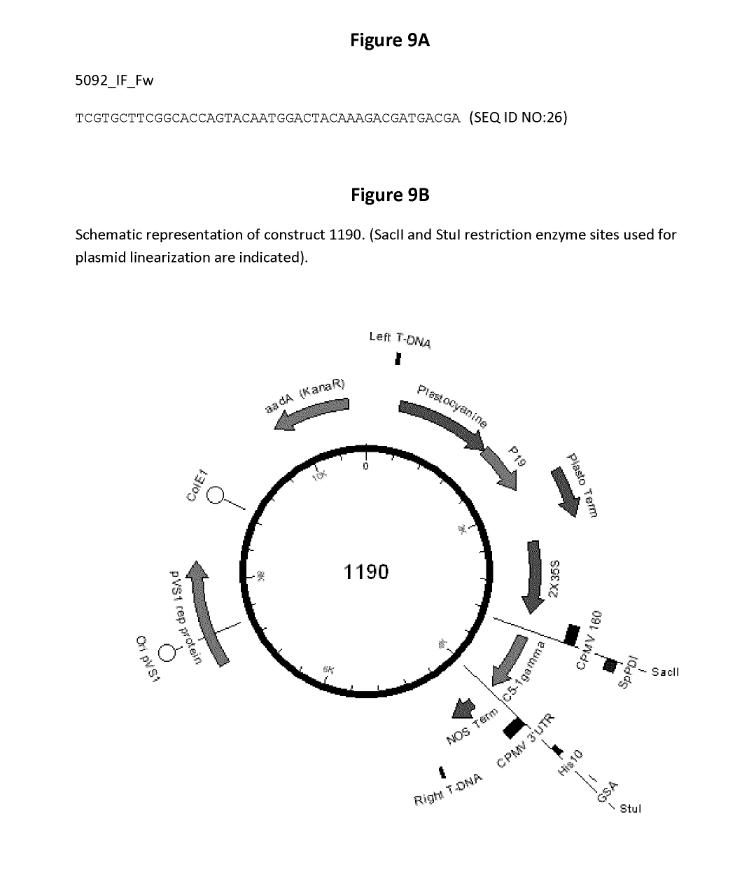

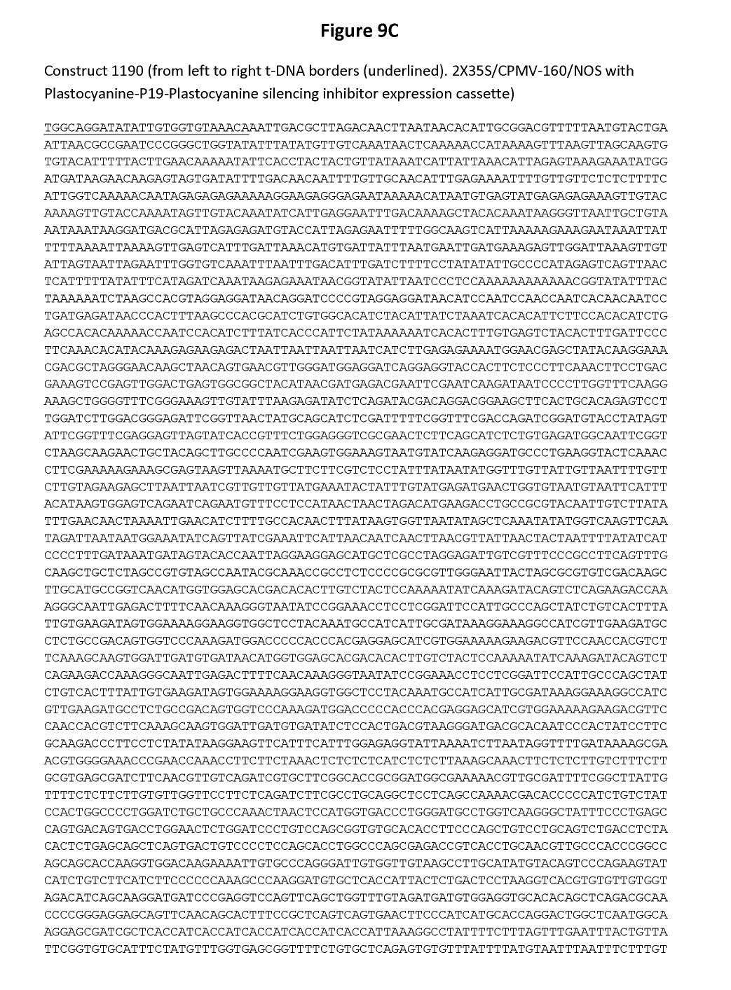

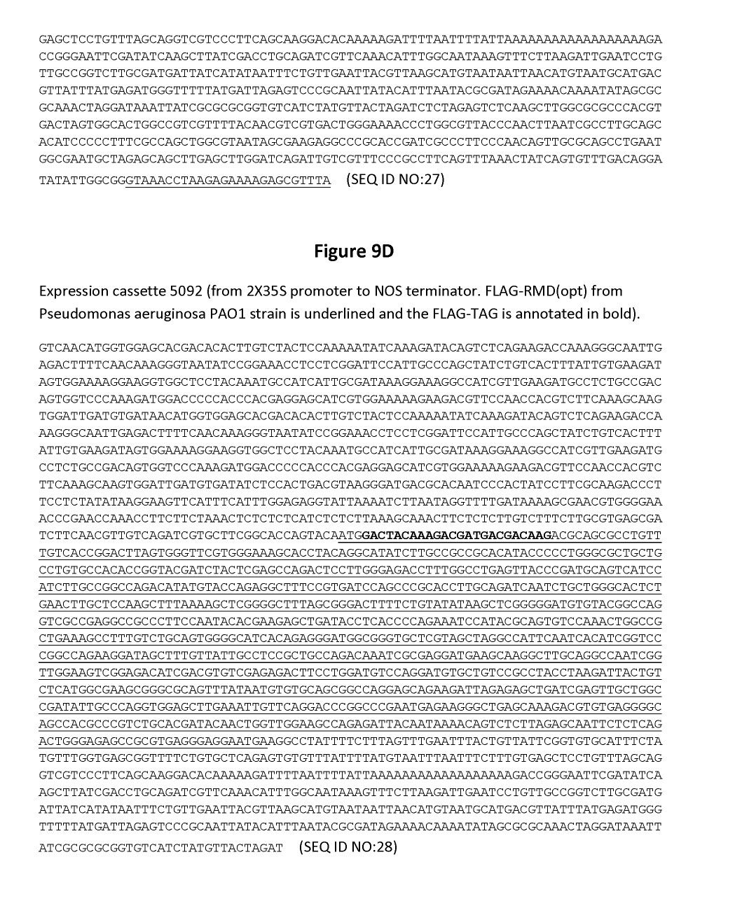

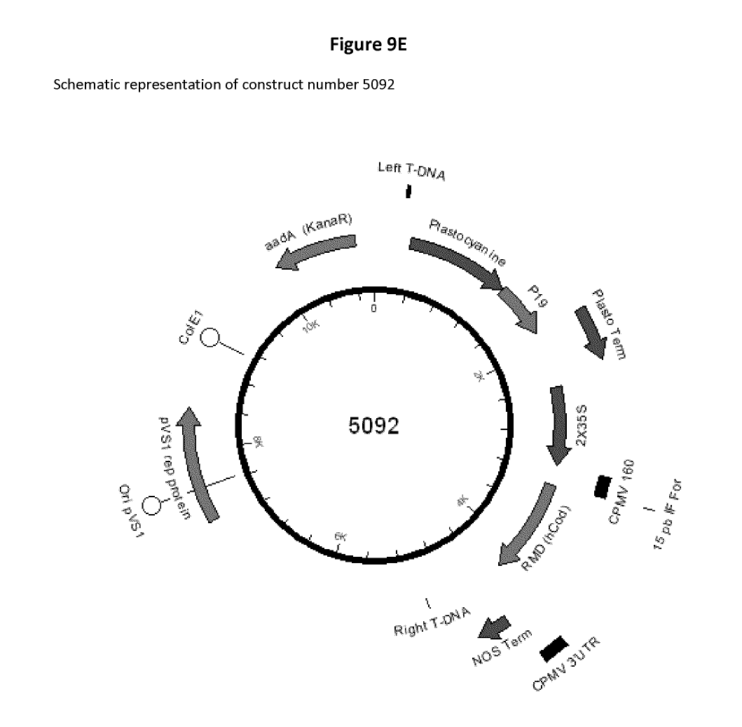

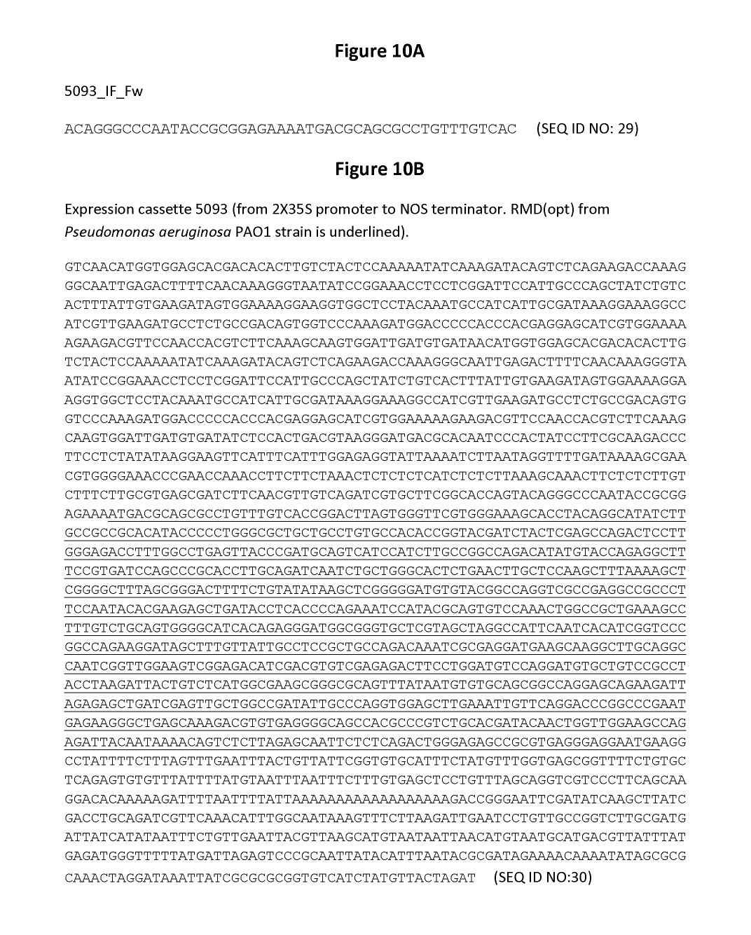

An optimized sequence encoding RMD from Pseudomonas aeruginosa strain PAO1 tagged in N-terminal with a FLAG in frame was cloned into 2X35S/CPMV 160/NOS expression system using the following PCR-based method. A fragment containing the RMD coding sequence was first amplified to add the FLAG tag in N-Ter using primers Flag_Rmd_Fw (FIG. 8A, SEQ ID NO: 19) and 5091_5092_IF_Rev (FIG. 8B, SEQ ID NO:20), using optimized RMD gene sequence (FIG. 8C, SEQ ID NO:21) as template. For sequence optimization, RMD protein sequence (Genbank accession number AAG08839.1) was backtranslated and optimized for human codon usage, GC content and mRNA structure. The PCR product was used as template for a second amplification using 5092_IF_Fw (FIG. 9A, SEQ ID NO:26) and 5091_5092_IF_Rev (FIG. 8B, SEQ ID NO:20). The final PCR product was cloned in 2X35S/CPMV 160/NOS expression system using In-Fusion cloning system (Clontech, Mountain View, Calif.). Construct number 1190 (FIG. 9B) was digested with SacII and StuI restriction enzyme and the linearized plasmid was used for the In-Fusion assembly reaction. Construct number 1190 is an acceptor plasmid intended for “In Fusion” cloning of genes of interest in a 2X35 S/CPMV 160/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 9C (SEQ ID NO:27). The resulting construct was given number 5092 (FIG. 9D, SEQ ID NO:28). The amino acid sequence of FLAG-Nter-RMD from Pseudomonas aeruginosa strain PAO1 is presented in FIG. 8H (SEQ ID NO:25). A representation of plasmid 5092 is presented in FIG. 9E.

2X35S/CPMV 160+/RMD(opt)/NOS (Construct number 5093; also termed: “160+/RMD” or “160+/RMD”)

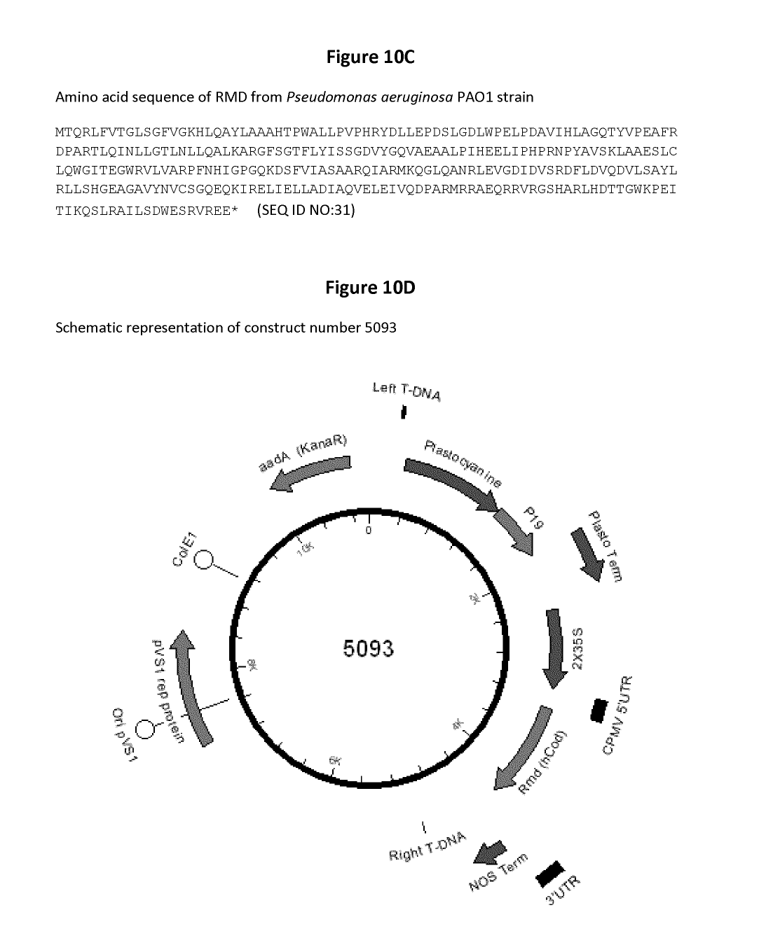

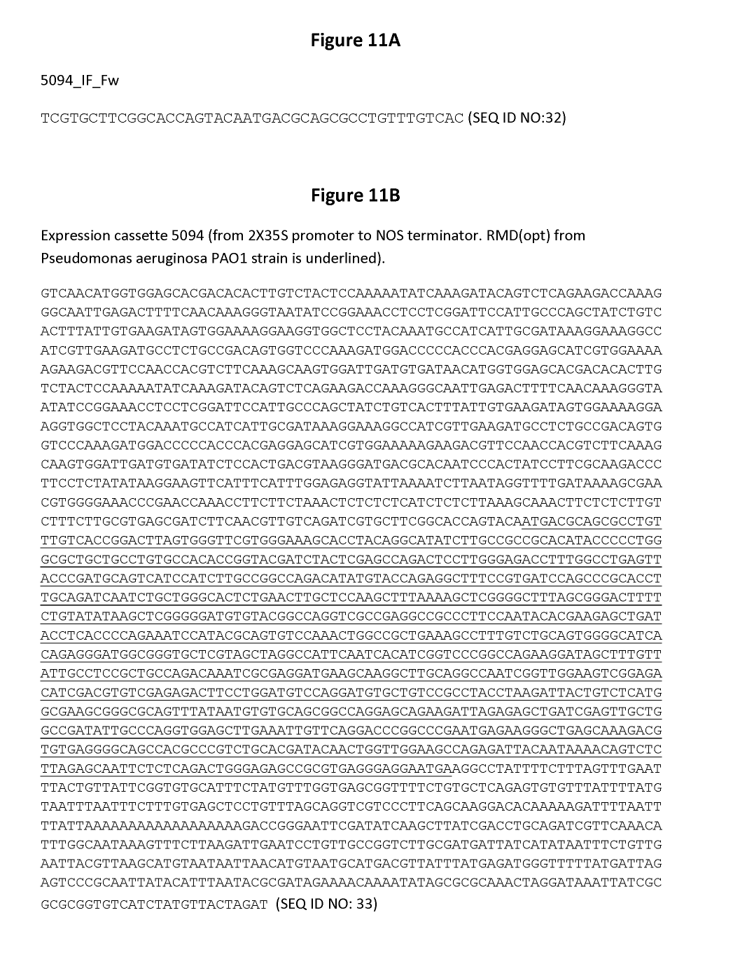

An optimized sequence encoding RMD from Pseudomonas aeruginosa strain PAO1 was cloned into 2X35S/CPMV 160+/NOS expression system using the following PCR-based method. A fragment containing the RMD coding sequence was amplified using primers 5093_IF_Fw (FIG. 10A, SEQ ID NO:29) and 5091_5092_IF_Rev (FIG. 8B, SEQ ID NO:20), using optimized RMD gene sequence (FIG. 8C, SEQ ID NO:21) as template. For sequence optimization, RMD protein sequence (Genbank accession number AAG08839.1) was backtranslated and optimized for human codon usage, GC content and mRNA structure. The PCR product was cloned in 2X35 S/CPMV 160+/NOS expression system using In-Fusion cloning system (Clontech, Mountain View, Calif.). Construct number 2171 (FIG. 8E) was digested with SacII and StuI restriction enzyme and the linearized plasmid was used for the In-Fusion assembly reaction. Construct number 2171 is an acceptor plasmid intended for “In Fusion” cloning of genes of interest in a 2X35 S/CPMV 160+/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 8F (SEQ ID NO:23). The resulting construct was given number 5093 (FIG. 10B, SEQ ID NO:30). The amino acid sequence of RMD from Pseudomonas aeruginosa strain PAO1 is presented in FIG. 10C (SEQ ID NO:31). A representation of plasmid 5093 is presented in FIG. 10D.

2X35 S/CPMV 160/RMD(opt)/NOS (Construct number 5094; also termed 160/RMD″) 160/RMD″)

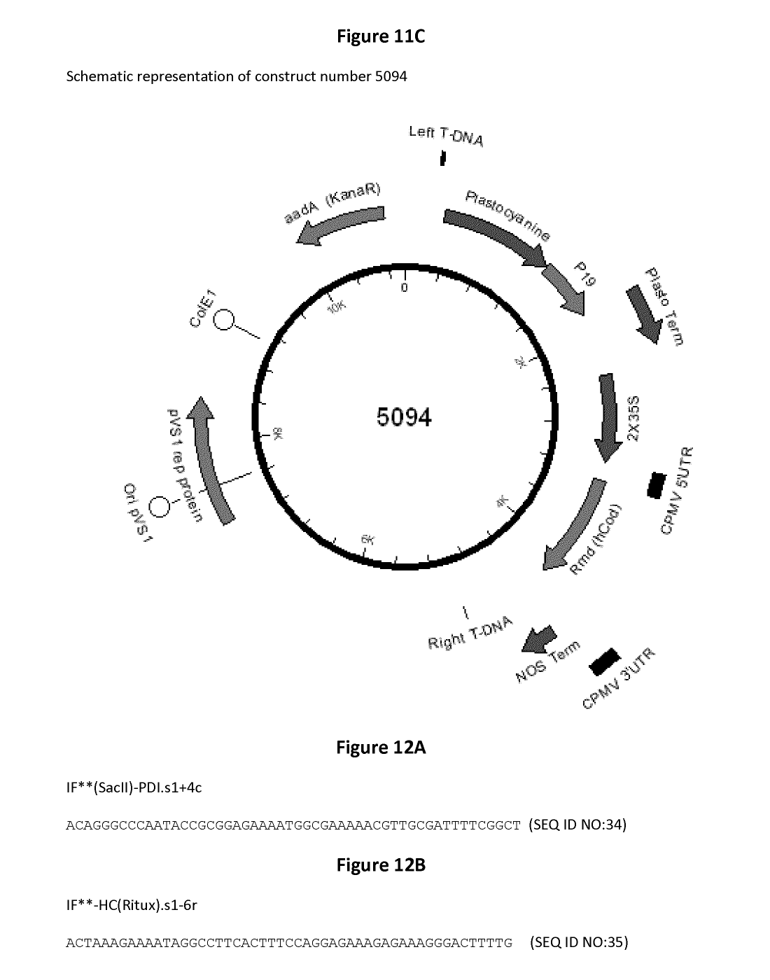

An optimized sequence encoding RMD from Pseudomonas aeruginosa strain PAO1 was cloned into 2X35S/CPMV 160/NOS expression system using the following PCR-based method. A fragment containing the RMD coding sequence was amplified using primers 5094_IF_Fw (FIG. 11A, SEQ ID NO:33) and 5091_5092 IF_Rev (FIG. 8B, SEQ ID NO:20), using optimized RMD gene sequence (FIG. 8C, SEQ ID NO:21) as template. For sequence optimization, RMD protein sequence (Genbank accession number AAG08839.1) was backtranslated and optimized for human codon usage, GC content and mRNA structure. The PCR product was cloned in 2X35S/CPMV-160/NOS expression system using In-Fusion cloning system (Clontech, Mountain View, Calif.). Construct number 1190 (FIG. 9B) was digested with SacII and StuI restriction enzyme and the linearized plasmid was used for the In-Fusion assembly reaction. Construct number 1190 is an acceptor plasmid intended for “In Fusion” cloning of genes of interest in a 2X35 S/CPMV 160/NOS expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 9C (SEQ ID NO:27). The resulting construct was given number 5094 (FIG. 11B, SEQ ID NO: 33). The amino acid sequence of RMD from Pseudomonas aeruginosa strain PAO1 is presented in FIG. 10C (SEQ ID NO:31). A representation of plasmid 5094 is presented in FIG. 11C.

A H-2X35 S/CPMV 160/atRMD(opt)/NOS (Construct Number 3431)

An optimized sequence encoding RMD from Agrobacterium tumefaciens strain TS43 was cloned into 2X35S/CPMV 160/NOS expression system using the following PCR-based method. A fragment containing the RMD coding sequence was amplified using primers IF-atRMD(opt).c (FIG. 15A, SEQ ID NO:44) and IF-atRMD(opt).r (FIG. 15B, SEQ ID NO: 45), using optimized RMD gene sequence (FIG. 15C, SEQ ID NO: 46) as template. For sequence optimization, RMD protein sequence (Genbank accession number WP_031234119.1) was backtranslated and optimized for human codon usage, GC content and mRNA structure. The PCR product was cloned in 2X35 S/CPMV-160/NOS expression system using In-Fusion cloning system (Clontech, Mountain View, Calif.). Construct number 1190 (FIG. 9B) was digested with SacII and StuI restriction enzyme and the linearized plasmid was used for the In-Fusion assembly reaction. Construct number 1190 is an acceptor plasmid intended for “In Fusion” cloning of genes of interest in a 2X35S/CPMV 160/NOS expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 9C (SEQ ID NO:27). The resulting construct was given number 3431 (FIG. 15D, SEQ ID NO:47). The amino acid sequence of RMD from Agrobacterium tumefaciens strain TS43 is presented in FIG. 15E (SEQ ID NO: 48). A representation of plasmid 3431 is presented in FIG. 15F.

I-2X35S/CPMV 160/pbRMD(opt)/NOS (Construct Number 3432)

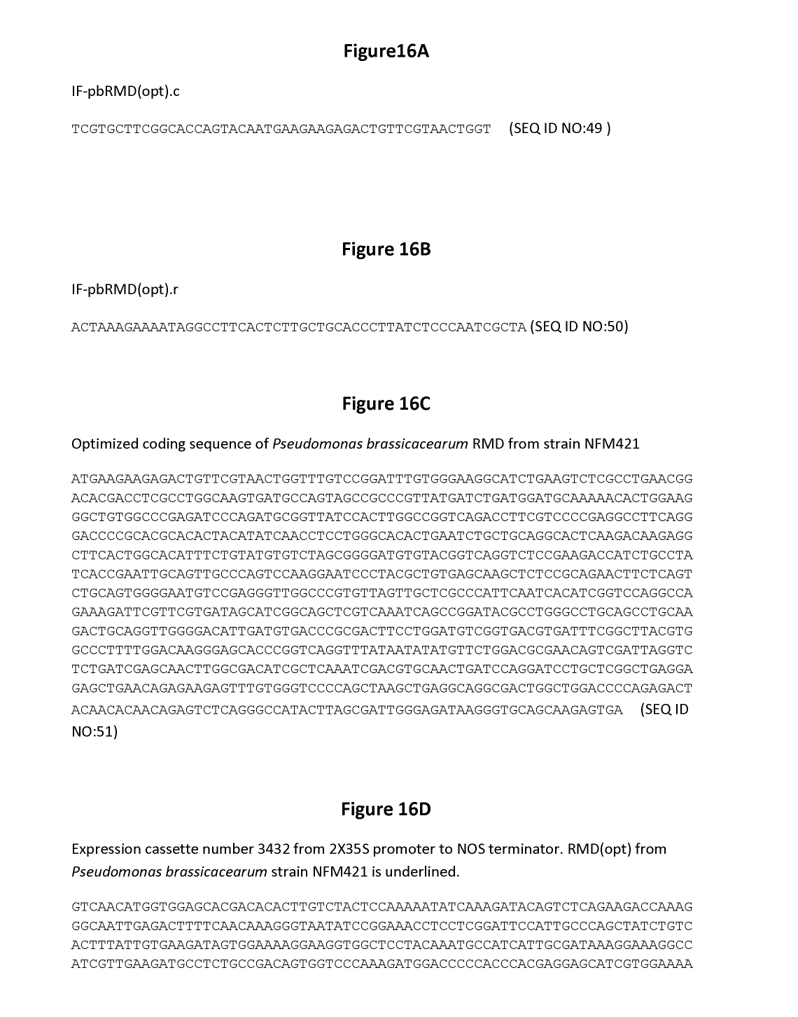

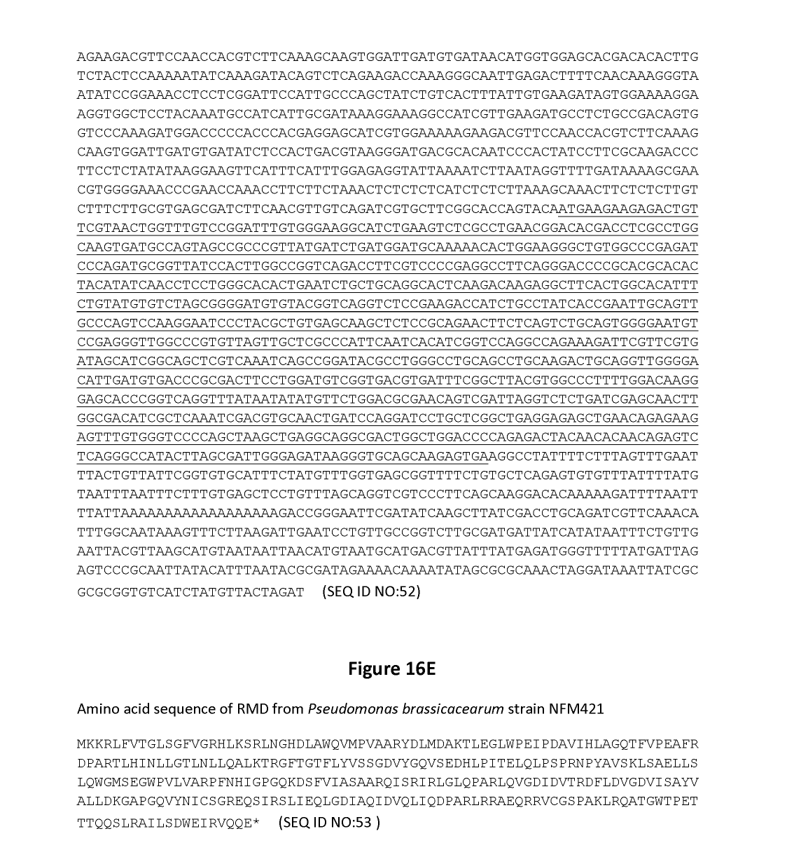

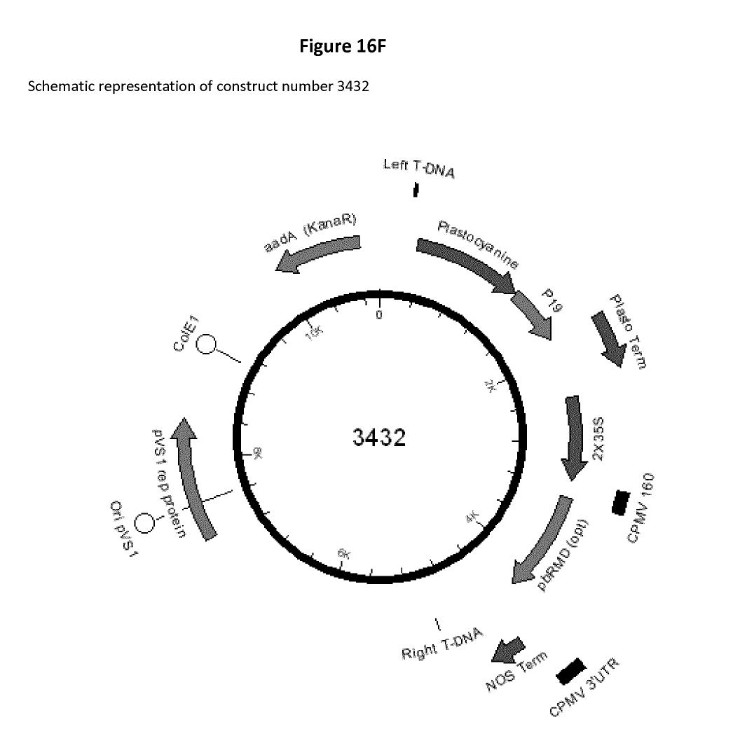

An optimized sequence encoding RMD from Pseudomonas brassicacearum strain NFM421 was cloned into 2X35S/CPMV 160/NOS expression system using the following PCR-based method. A fragment containing the RMD coding sequence was amplified using primers IF-pbRMD(opt).c (FIG. 16A, SEQ ID NO:49) and IF-pbRMD(opt).r (FIG. 16B, SEQ ID NO:50), using optimized RMD gene sequence (FIG. 16C, SEQ ID NO:51) as template. For sequence optimization, RMD protein sequence (Genbank accession number WP_013694623.1) was backtranslated and optimized for human codon usage, GC content and mRNA structure. The PCR product was cloned in 2X35S/CPMV-160/NOS expression system using In-Fusion cloning system (Clontech, Mountain View, Calif.). Construct number 1190 (FIG. 9B) was digested with SacII and StuI restriction enzyme and the linearized plasmid was used for the In-Fusion assembly reaction. Construct number 1190 is an acceptor plasmid intended for “In Fusion” cloning of genes of interest in a 2X35S/CPMV 160/NOS expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 9C (SEQ ID NO:27). The resulting construct was given number 3432 (FIG. 16D, SEQ ID NO:52). The amino acid sequence of RMD from Pseudomonas brassicacearum strain NFM421 is presented in FIG. 16E (SEQ ID NO:53). A representation of plasmid 3432 is presented in FIG. 16D.

J-2X35S/CPMV 160/psRMD(opt)/NOS (Construct Number 3433)

An optimized sequence encoding RMD from Pseudomonas syringae was cloned into 2X35S/CPMV 160/NOS expression system using the following PCR-based method. A fragment containing the RMD coding sequence was amplified using primers IF-psRMD(opt).c (FIG. 17A, SEQ ID NO:54) and IF-psRMD(opt).r (FIG. 17B, SEQ ID NO:55), using optimized RMD gene sequence (FIG. 17C, SEQ ID NO:56) as template. For sequence optimization, RMD protein sequence (Genbank accession number WP_010430271.1) was backtranslated and optimized for human codon usage, GC content and mRNA structure. The PCR product was cloned in 2X35S/CPMV-160/NOS expression system using In-Fusion cloning system (Clontech, Mountain View, Calif.). Construct number 1190 (FIG. 9B) was digested with SacII and StuI restriction enzyme and the linearized plasmid was used for the In-Fusion assembly reaction. Construct number 1190 is an acceptor plasmid intended for “In Fusion” cloning of genes of interest in a 2X35S/CPMV 160/NOS expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 9C (SEQ ID NO:27). The resulting construct was given number 3433 (FIG. 17D, SEQ ID NO:57). The amino acid sequence of RMD from Pseudomonas syringae is presented in FIG. 17E (SEQ ID NO:58). A representation of plasmid 3433 is presented in FIG. 17F.

K-2X35 S/CPMV 160/xvRMD(opt)/NOS (Construct Number 3434)

An optimized sequence encoding RMD from Xanthomonas vasicola strain NCPPB1326 was cloned into 2X35 S/CPMV 160/NOS expression system using the following PCR-based method. A fragment containing the RMD coding sequence was amplified using primers IF-xvRMD(opt).c (FIG. 18A, SEQ ID NO:59) and IF-xvRMD(opt).r (FIG. 18B, SEQ ID NO:60), using optimized RMD gene sequence (FIG. 18C, SEQ ID NO:61) as template. For sequence optimization, RMD protein sequence (Genbank accession number WP_010371840.1) was backtranslated and optimized for human codon usage, GC content and mRNA structure. The PCR product was cloned in 2X35S/CPMV-160/NOS expression system using In-Fusion cloning system (Clontech, Mountain View, Calif.). Construct number 1190 (FIG. 9B) was digested with SacII and StuI restriction enzyme and the linearized plasmid was used for the In-Fusion assembly reaction. Construct number 1190 is an acceptor plasmid intended for “In Fusion” cloning of genes of interest in a 2X35 S/CPMV 160/NOS expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in FIG. 9C (SEQ ID NO:27). The resulting construct was given number 3434 (FIG. 18D, SEQ ID NO:62). The amino acid sequence of RMD from Xanthomonas vasicola strain NCPPB 1326 is presented in FIG. 18E (SEQ ID NO:63). A representation of plasmid 3434 is presented in FIG. 18F).

### Agrobacterium Transfection

Agrobacterium strain AGL1 was transfected by heat shock transformation with the DNA constructs using the methods described by Höfgen R et Willmitzer L 1988 (Nucleic Acids Research October 25; 16(20):9877). Transfected Agrobacterium were grown in 200 ml BBLselect APS medium (Becton, Dickinson and Company, NJ) supplemented with 10 mM 2-(N-morpholino) ethanesulfonic acid (MES), 50 μg/ml kanamycin and 25 μg/ml of carbenicillin pH5.6 to an OD600 between 3.0 and 4.0. Agrobacterium suspensions were diluted before use in infiltration medium (10 mM MgCl2 and 10 mM MES pH 5.6).

### Preparation of Plant Biomass, Inoculum and Agroinfiltration

The terms “biomass” and “plant matter” as used herein are meant to reflect any material derived from a plant. Biomass or plant matter may comprise an entire plant, tissue, cells, or any fraction thereof. Further, biomass or plant matter may comprise intracellular plant components, extracellular plant components, liquid or solid extracts of plants, or a combination thereof. Further, biomass or plant matter may comprise plants, plant cells, tissue, a liquid extract, or a combination thereof, from plant leaves, stems, fruit, roots or a combination thereof. A portion of a plant may comprise plant matter or biomass.

Nicotiana benthamiana plants were grown from seeds in flats filled with a commercial peat moss substrate. The plants were allowed to grow in a growth chamber under a 16/8 photoperiod and a temperature regime of 26° C. day/24° C. night. Three weeks after seeding, individual plantlets were picked out, transplanted in pots and left to grow in the growth chamber for three additional weeks under the same environmental conditions.

Agrobacteria transfected with each construct were grown in a BBLselect APS medium supplemented with 10 mM 2-(N-morpholino)ethanesulfonic acid (MES), 50 μg/ml kanamycin and 25 μg/ml of carbenicillin pH5.6 until they reached an OD600 between 3.0 and 4.0. Agrobacterium suspensions were stored overnight at 4° C. On the day of infiltration, culture batches were diluted in infiltration medium to reach an appropriate final OD600 and allowed to warm before use. Whole plants of N. benthamiana were placed upside down in the bacterial suspension in an air-tight stainless steel tank under a vacuum of 50 Torr for 1-min. Plants were returned in a growth chamber for a 3-6 day incubation period until harvest, under the same environmental conditions as growth and with a control of the hygrometry of 70%.

### Leaf Harvest and Total Protein Extraction

Following incubation, the aerial part of plants was harvested, frozen at −80° C. and crushed into pieces. Total soluble proteins were extracted by homogenizing (Polytron) each sample of frozen-crushed plant material in 2 volumes of cold 150 mM Tris pH 7.6, 150 mM NaCl, 1 mM phenylmethanesulfonyl fluoride and 4 mg/ml Metabisulfite. After homogenization, the slurries were centrifuged at 20,000 g for 20 min at 4° C. and these clarified crude extracts (supernatant) kept for analyses.

### Protein Analysis and Immunoblotting

The total protein content of clarified crude extracts was determined by the Bradford assay (Bio-Rad, Hercules, Calif.) using γ-globuline as the reference standard. Appropriate quantities of proteins (0.25 to 2 μg total soluble proteins) were separated by SDS-PAGE and electrotransferred onto polyvinylene difluoride (PVDF) membranes (Bio-Rad, Hercules, Calif.) for immunodetection. Prior to immunoblotting, the membranes were blocked with 5% skim milk and 0.1% Tween-20 in Tris-buffered saline (TBS-T) for 16-18h at 4° C.

For the detection of al-3 Fucose, Immunoblotting was performed with a first incubation with a primary antibody Anti-Fucose (Agrisera AS07 268) diluted at 1:10000 in 2% skim milk in TBS-Tween 20 0.1%. Secondary antibody used for chemiluminescence detection was a Goat Anti-Rabbit (Sigma A0545) diluted at 1:80000 in 2% skim milk in TBS-Tween 20 0.1%. Immunoreactive complexes were detected by chemiluminescence using luminol as the substrate (Bio-Rad, Hercules, Calif.). The membrane was dehybridized by incubation in NaOH 1M for 8 minutes and then rinsed three times in wash solution to allow reprobing using anti-IgG antibody.

For the detection of recombinant C2B8 antibodies, a donkey anti-Human IgG/POD (Jackson Immunoreseach 709-035-149) antibody was diluted at 1:7500 in 2% skim milk in TBS-Tween 20 0.1%. Immunoreactive complexes were detected by chemiluminescence using luminol as the substrate (Bio-Rad, Hercules, Calif.).

For detection and quantification of fucosylation of rituximab monoclonal antibody, all immunoblotting images were analyzed using ImageLab software (Bio-Rad, Hercules, Calif.). After band density analysis on images from western blot probed with anti-fucose and reprobed with anti-IgG1after membrane dehybridation, fucosylation of rituximab was measured and compared with a control condition. All values were adjusted using IgG1 quantity in regards to the control condition and reported as % of the control condition.

### Antibody Purification for MS Analysis

Following incubation, the aerial part of plants was harvested, frozen at −80° C. and crushed into pieces. Total soluble proteins were extracted by homogenizing (Polytron) each sample of frozen-crushed plant material in 2 volumes of cold 150 mM Tris pH 7.6, 150 mM NaCl, 1 mM phenylmethanesulfonyl fluoride and 4 mg/ml Metabisulfite. After homogenization, the slurries were first filtered through four layers of Miracloth, then centrifuged at 20,000 g for 20 min at 4° C. and filtered at 0.45 m then at 0.2 m before purify the human antibody C2B8 using the Prosep®-A kit (Merck Millipore). After elution, the purified antibody was conserved at −20° C. for analyses. N-glycopeptides analysis was performed by mass spectrometry (LC ESI MS/MS; as described in Li et. al. (2015, Plt. Biotech. J., pp. 1-10).

### Example 1: Expression of Flag-RMD and RMD in N. benthamiana Plants

Expression of Flag-RMD in N. benthamiana Plant and Co-Expression with Rituximab Monoclonal Antibody

The expression of GDP-4-dehydro-6-deoxy-D-mannose reductase (RMD) from Pseudomonas aeruginosa fused to a Flag-TAG (Flag-RMD) under the control of CPMV 160+(160+/Flag-RMD; construct no 5091) or CPMV 160 (160/Flag-RMD; construct no 5092) expression system in N. benthamiana was tested using agroinfiltration.

FIG. 2 shows the soluble protein content (SDS-PAGE) of crude extract from N. benthamiana plants agroinfiltrated with construct 5091 or 5092 at an OD600 of 0.4 (i.e. the amount of bacterial vector supplied to the plant during agroinfiltration), and expressing only the Flag-RMD. A strong band can be seen at the expected molecular weight of the Flag-RMD (34.9 kDa) which is not present in the negative control (crude extract of agro-infiltrated empty vector). Expression using either the CPMV 160+ or the CPMV160 enhancer element led to high expression of the enzyme.

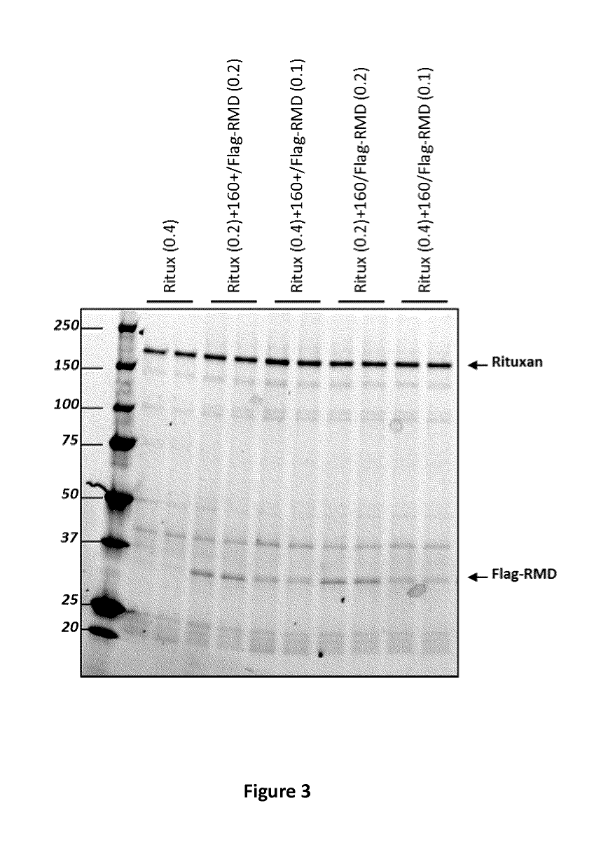

FIG. 3 presents the soluble protein content (SDS-PAGE) of crude extract from N. benthamiana plants agroinfiltrated with rituximab monoclonal antibody (construct 5072) at an OD600 of 0.2 or 0.4 (i.e. a relative indication of the amount of bacterial vector supplied to the plant during agroinfiltration) and co-infiltrated with construct 5091 or 5092 at an OD600 of 0.1 or 0.2 (the amount of bacterial vector supplied to the plant during agroinfiltration).

Co-expression of Flag-RMD with rituximab did not impact rituximab accumulation.

FIG. 3 also shows that the concentration of amount of Flag-RMD construct used during infiltration is related to RMD accumulation within the plant. For example, reduced band intensity was observed when using 0.1 OD600 (the amount of bacterial vector supplied to the plant during agroinfiltration) instead of 0.2 OD600.

Expression of RMD in N. benthamiana Plant and Co-Expression with Rituximab Monoclonal Antibody

The expression of GDP-4-dehydro-6-deoxy-D-mannose reductase from Pseudomonas aeruginosa (RMD) under the control of CPMV 160+(160+/RMD; construct no 5093; also referred to as 160+/paRMD)) or CPMV 160 (160/RMD; construct no 5094; also referred to as 160/paRMD) expression system in N. benthamiana was tested by agroinfiltration.

FIG. 5 presents the soluble protein content (coomassie-stained SDS-PAGE) of crude extract from N. benthamiana plants agroinfiltrated with construct 5093 or 5094 at an OD600 of 0.4 (the amount of construct supplied to the plant during agroinfiltration) and expressing only RMD, or agroinfiltrated with rituximab monoclonal antibody (construct 5072) at an OD600 of 0.4 (the amount of construct supplied to the plant during agroinfiltration) and co-infiltrated with construct 5093 or 5094 at an OD600 of 0.15.

As shown in FIG. 5, a strong band can be seen at the expected molecular weight of the RMD (33.9 kDa) which is not present in the negative control (crude extract of agro-infiltrated empty vector) and that either expression system (using CPMV 160+ or CPMV 160) led to high expression of the enzyme. The use of the CPMV 160+ enhancer resulted in slightly higher paRMD accumulation when paRMD was expressed alone, or when paRMD was co-expressed with rituximab mAb. When paRMD was co-expressed with rituximab, paRMD did not have any impact on rituximab accumulation.

### Example 2: Effect of Flag-RMD or RMD Co-Expression on Rituximab Fucosylation in Wild-Type Plants

The effect of the co-expression of Flag-RMD on rituximab fucosylation on antibody N-glycans was assessed by western blot analysis using anti-fucose. After detection by the anti-fucose, membranes were dehybridized and reprobed with anti-IgG1 for normalization of IgG loads quantity.

FIG. 4 presents the anti-fucose (upper panel) and anti-IgG1 (lower panel) western blot analysis of crude extract from N. benthamiana plants agroinfiltrated with rituximab monoclonal antibody (construct 5072) at an OD600 of 0.2 or 0.4 (the amount of bacterial vector supplied to the plant during agroinfiltration) and co-infiltrated with construct 5091 or 5092 at an OD600 of 0.1 or 0.2. As seen in FIG. 4, no reduction of rituximab fucosylation is observed when Flag-RMD was co-expressed with rituximab. The concentration of Flag-RMD (i.e. OD600 amount of bacterial vector used for agroinfiltration) or expression system (CPMV 160+ or CPMV 160) had no effect on rituximab fucosylation.

The effect of the co-expression of RMD (with no Flag sequence) on rituximab fucosylation on antibody N-glycans was assessed by western blot analysis using the same method as described above.

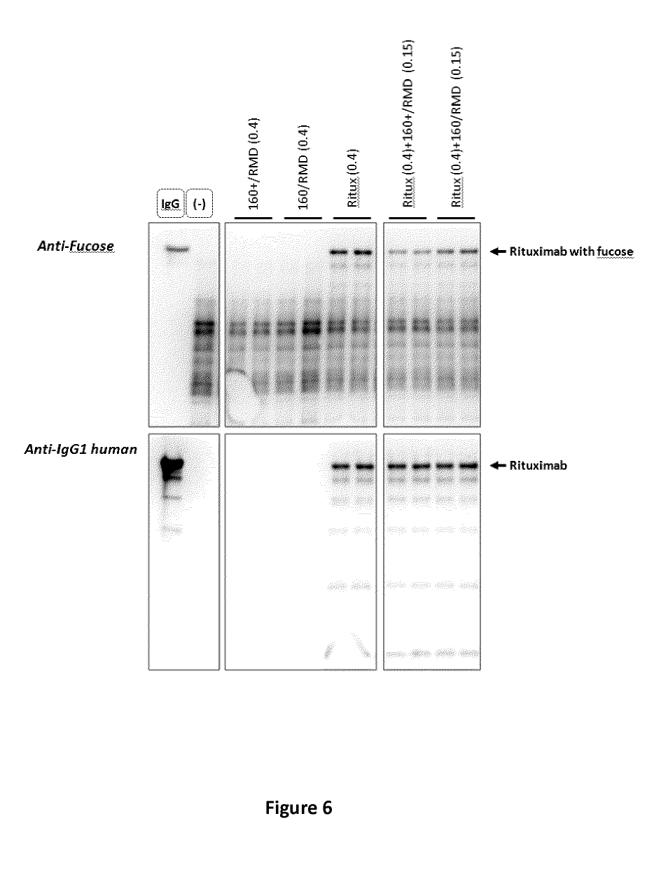

FIG. 6 presents the anti-fucose (upper panel) and anti-IgG1 (lower panel) western blot analysis of crude extract from N. benthamiana plants agroinfiltrated with rituximab monoclonal antibody (construct 5072) at an OD600 of 0.4 (the amount of bacterial vector supplied to the plant during agroinfiltration) alone or co-infiltrated with construct 5093 (160+/paRMD) or 5094 (160/paRMD) at an OD600 of 0.15. As seen in FIG. 6, a reduction of rituximab fucosylation is observed when paRMD was co-expressed with rituximab. paRMD expressed using either CPMV 160+ or CPMV 160 lead to reduced rituximab fucosylation. The paRMD expressed under CPMV 160+ resulted in a greater reduction of fucosylation. Table 3 below summarizes the densitometry analysis rituximab fucosylation from FIG. 6.

TABLE 3
Characterization of rituximab fucosylation co-expressed in the
presence or absence of paRMD (RMD) on IgG N-Glycan by
densitometry analysis using anti-fucose (normalization, set at
100%, with IgG load quantity).

| | % anti IgG1 | % anti | % Fucose |
|---|---|---|---|
| Condition | human | Fucose | normalised |
| Ritux (0.4) | 100% | 100% | 100% |
| Ritux (0.4) + 160+/RMD (0.15) | 97% | 34% | 35% |
| Ritux (0.4) + 160/RMD (0.15) | 99% | 63% | 63% |

As seen in table 3, fucosylation of rituximab antibody co-expressed with 160+/RMD was reduced by 65% (35% of the control level) when compared to rituximab expressed alone. When using 160/RMD, fucosylation was reduced by 37% (63% of the control level).

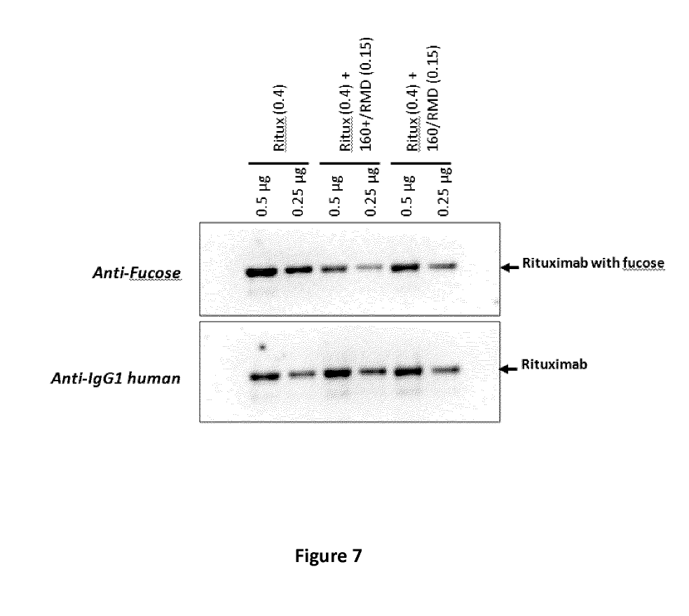

These results were confirmed by reanalysis of new crudes from alternative extracts, loaded onto the gel at two protein-loading quantities (0.5 and 0.25 μg total soluble protein). Western blot analysis is presented in FIG. 7 and densitometry analysis is presented in the table 4 below. Compared to the above analysis, a slightly higher reduction in fucosylation was found for both 160+/RMD (24% of the control level; fucosylation reduced by 76%) and 160/RMD (47% of the control level; fucosylation reduced by 53%). Values for rituximab expressed alone (% anti hIgG1, % anti fucose, % fucose normalized), loaded at 0.5 μg TSP were set at 100% (shaded grey).

TABLE 4
Characterization of rituximab fucosylation co-expressed in the
presence or absence of 160+/paRMD (160+/RMD) or
160/paRMD (160/RMD) on IgG N-Glycan by densitometry
analysis using anti-fucose (normalization, set at 100%,
with IgG 0.5 μg TSP load).

| | TSP | % anti | % anti | % Fucose | Mean % |
|---|---|---|---|---|---|
| Condition | (μg) | hIgG1 | Fucose | normalised | Fucose |
| Ritux (0.4) | 0.5 | 100% | 100% | 100% | 99% |
| | 0.25 | 54% | 53% | 98% | |
| Ritux (0.4) + | 0.5 | 124% | 33% | 26% | 24% |
| 160+/RMD (0.15) | 0.25 | 67% | 15% | 22% | |
| Ritux (0.4) + | 0.5 | 112% | 55% | 49% | 47% |
| 160/RMD (0.15) | 0.25 | 59% | 27% | 45% | |

The results presented in FIGS. 4 and 6, and Tables 3 and 4 suggest that the Flag sequence may interfere with RMD activity. As a result, the RMD sequence, for use as described herein, preferably does not comprise a Flag sequence.

### Example 3: Effect of RMD Co-Expression on Rituximab Glycan Profile (Fucosylation) in Wild-Type Plants and in Fuct/XylT Knockout Plants

### Glycan Profile—Wild-Type Plants

The rituximab antibody (construct 5072) was transiently expressed in wild-type Nicotiana benthamiana plants with and without the co-expression of 160+/RMD (construct 5093; paRMD) or 160/RMD (construct 5094; paRMD) and purified as described above in example 2. N-Glycan profiling and analysis of glycopeptides of the purified antibodies was characterized using MS (LC ESI MS/MS; as described in Li et. al. (2015, Plt. Biotech. J., pp. 1-10). The N-glycosylation profile on a unique site (N301) of purified rituximab antibodies was compared to that of wild-type plants. The results are presented in Table 5, below.

TABLE 5
Comparison of N-glycan profile of the purified rituximab antibody produced in wild-type plants, with and without co-expression of paRMD
under the control of CPMV 160+ or CPMV 160 expression system. Bacterial vector comprising rituximab was infiltrated at an OD600 of 0.5
while the bacterial vector comprising paRMD, when present, was infiltrated at an OD of 0.25. Numbers represent the average percentage
of each glycoform indentified from each condition.

| | 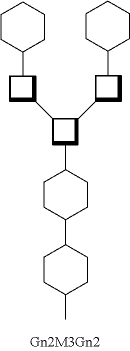 | 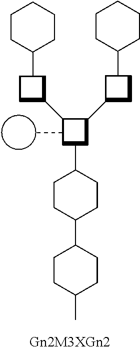 | 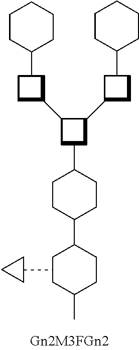 | 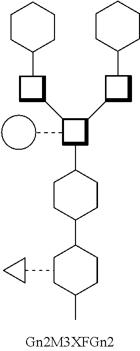 | 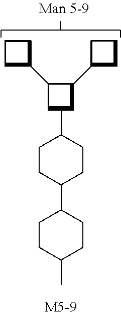 |
|---|---|---|---|---|---|
| Wild-type | 4 | 2 | 4 | 88 | 2 |
| Wild-type + 160+/RMD | 26 | 54 | 0 | 19 | 1 |
| Wild-type + 160/RMD | 20 | 28 | 1 | 47 | 2 |

Hexagon: N-acetylglucosamine; Square: mannose; Circle: xylose; Triangle: Fucose

A reduction in fucosylation was observed in plants co-expressing rituximab and 160+/RMD or 160/RMD consistent with the densitometry analysis presented in FIGS. 4 and 6 and Tables 3 and 4. When produced in wild-type plants, 92% of N-glycans in the antibody had α(1,3)-fucose (the sum of percentages of glycoforms Gn2M3FGn2 and Gn2M3XFGn2) and 8% of the N-glycans did not have α(1,3)-fucose (the sum of percentages of glycoforms Gn2M3Gn2, Gn2M3XGn2 and M5-9). In contrast, glycosylation analysis of plants comprising 160+/RMD revealed that 81% of the N-glycans did not have α(1,3)-fucose, 19% were α(1,3)-fucose-containing N-glycans. For plants expressing 160/RMD 50% of the N-glycans did not have α(1,3)-fucose, 48% of the N-glycans contained α(1,3)-fucose (see Table 5).

### Glycan Profile—FucT/XylT Knockout Plants

The rituximab antibody (construct 5072) was also transiently expressed in wild-type or knocked-out Nicotiana benthamiana plants lines (plant line NB14-29aT2; WO 2014/071039; Li et. al. 2015, Plt. Biotech. J. p 1-10; which are incorporated herein by reference) with and without the co-expression of 160+/RMD (construct 5093) or 160/RMD (construct 5094) and purified as described above. In the Cellectis plants two (1,3)-fucosyltransferase (FucT) genes and two β(1,2)-xylosyltransferase (XylT) genes have been knocked out.

N-Glycan profiling and analysis of glycopeptides of the purified antibodies was characterized using MS (LC ESI MS/MS; as described in Li et. al. (2015, Plt. Biotech. J., pp. 1-10). The results are presented in table 6.

TABLE 6
Comparison of N-glycan profile of the purified rituximab antibody produced in wild-type plants and FucT/XylT knockout (NB14-29aT2)
plants, with and without co-expression of RMD under the control of CPMV 160+ or CPMV 160 expression system. The bacterial vector comprising
rituximab was infiltrated at an OD600 of 0.5 while the bacterial vector comprising RMD, when present, was infiltrated at an OD of 0.25.
Numbers represent the average percentage of each glycoform identified from each plant line.

| | 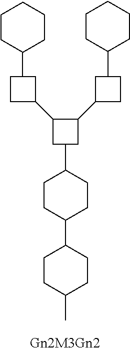 | 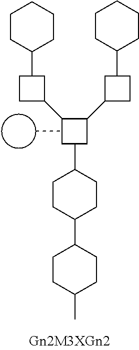 | 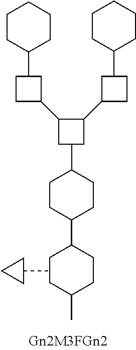 | 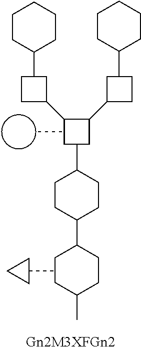 | 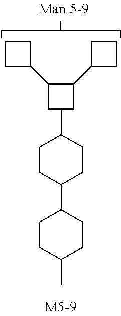 |
|---|---|---|---|---|---|
| Wild-type plants | 6 | 4 | 4 | 76 | 4 |
| Wild-type plants + | 20 | 39 | 0 | 34 | 2 |
| 160+/RMD | | | | | |
| Wild-type plants + | 24 | 40 | 0 | 30 | 2 |
| 160/RMD | | | | | |
| FucT/XylT | 60 | 0 | 37 | 0 | 1 |
| knockout plants | | | | | |
| FucT/XylT knockout | 90 | 0 | 10 | 0 | 0 |
| plants + 160+/RMD | | | | | |
| FucT/XylT knockout | 91 | 0 | 9 | 0 | 0 |
| plants + 160/RMD | | | | | |

Hexagon: N-acetylglucosamine; Square: mannose; Circle: xylose; Triangle: Fucose

Glycosylation analysis of the FucT/XylT knockout plants (plant line NB14-29aT2; WO 2014/071039; Li et. al. 2015, Plt. Biotech. J. p 1-10; which are incorporated herein by reference) showed that 61% of the N-glycans did not have α(1,3)-fucose (the sum of percentages of glycoforms Gn2M3FGn2 and M5-9), 37% were α(1,3)-fucose-containing N-glycans (glycoform Gn2M3FGn2). When 160/RMD was expressed in these plants 91% of the N-glycans did not have α(1,3)-fucose, and only 9% were α(1,3)-fucose-containing N-glycans. Similar results were achieved when 160+/RMD was expressed in FucT/XylT knockout plants. 90% of the N-glycans did not have α(1,3)-fucose, and only 10% were α(1,3)-fucose-containing N-glycans (see Table 6).

To test the effect of RMD quantity on fucosylation reduction efficacy, the rituximab antibody (construct 5072) was transiently expressed in wild-type or FucT/XylT knock-out Nicotiana benthamiana plants lines (NB14-29aT2) with and without the co-expression of 160/RMD (construct 5094) at various concentrations. The rituximab antibody was purified as described above.

N-Glycan profiling and analysis of glycopeptides of the purified antibodies was characterized using LC ESI MS/MS; as described in Li et. al. (2015, Plt. Biotech. J., pp. 1-10). The results are presented in table 7.

TABLE 7
Comparison of N-glycan profile of the rituximab antibody produced in wild-type plants and plants in which the two (1,3)-fucosyltransferase
(FucT) genes and the two β(1,2)-xylosyltransferase (XylT) genes have been knocked out (NB14-29aT2 plants). Rituximab was co-expressed with
various concentrations of 160/RMD construct in either wild-type plans or knock-out plants. Rituximab expressing construct was infiltrated
at an OD600 of 0.5 while the OD600 of RMD expressing construct, when present, is indicated between parentheses. Numbers
represent the average percentage of each glycoform identified from each plant line.

| | 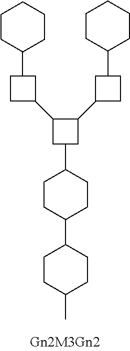 | 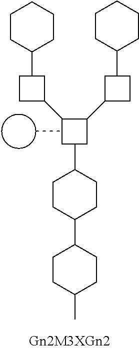 | 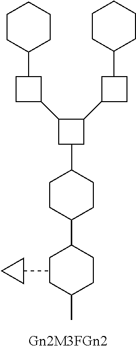 | 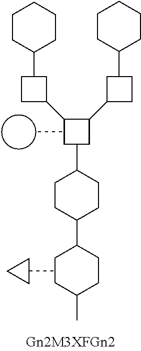 | 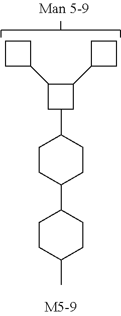 |
|---|---|---|---|---|---|
| Wild-type plants | 5 | 10 | 5 | 70 | 10 |
| Wild-type | 15 | 37 | 2 | 39 | 7 |
| plants + 160/RMD | | | | | |
| (0.25) | | | | | |
| FucT/XylT | 46 | 0 | 39 | 0 | 11 |
| knockout plants | | | | | |
| FucT/XylT | 62 | 0 | 28 | 0 | 9 |
| knockout plants + | | | | | |
| 160+/RMD (0.15) | | | | | |
| FucT/XylT | 69 | 0 | 21 | 0 | 7 |
| knockout plants + | | | | | |
| 160/RMD (0.25) | | | | | |
| FucT/XylT | 81 | 0 | 12 | 0 | 5 |
| knockout plants + | | | | | |
| 160/RMD (0.5) | | | | | |

Hexagon: N-acetylglucosamine; Square: mannose; Circle: xylose; Triangle: Fucose

The reduction in fucosylation (i.e a decrease in Gn2M3FGn2 glycoform, and an increase in the Gn2M3Gn2 glycoform) is observed in FucT/XylT knockout (NB14-29aT2) plants with an increase in the concentration of agroinfiltrated RMD.

Glycosylation analysis of the rituximab produced in FucT/XylT knockout plants indicates that 57% of the N-glycans did not have α(1,3)-fucose (glycoforms Gn2M3Gn2 and M5-9), and 39% were α(1,3)-fucose-containing N-glycans (Gn2M3FGn2). Co-expression of 160/RMD with rituximab in the knock-out plants at an OD600 of 0.15, 71% of the N-glycans did not have α(1,3)-fucose, and 28% were α(1,3)-fucose-containing N-glycans. When 160/RMD was co-expressed with rituximab in these plants at an OD600 of 0.25, 76% of the N-glycans did not have α(1,3)-fucose and 21% were α(1,3)-fucose-containing N-glycans and, when using an OD600 of 0.5, 86% of the N-glycans did not have α(1,3)-fucose, and 12% were α(1,3)-fucose-containing N-glycans.

### Example 4: Expression of atRMD, pbRMD, psRMD and xvRMD in Plants

The expression of GDP-4-dehydro-6-deoxy-D-mannose reductase from Agrobacterium tumefaciens (atRMD) under the control of CPMV 160 (160/atRMD, construct no 3431), from Pseudomonas brassicacearum (pbRMD) under the control of CPMV 160 (160/pbRMD; construct no 3432), from Pseudomonas syringae (psRMD) under the control of CPMV 160 (160/psRMD, construct no 3433) and from Xanthomonas vasicola (xvRMD) under the control of CPMV 160 (160/xvRMD, construct no 3434) in N. benthamiana was first tested by agroinfiltration. FIG. 7 presents the crude extract analysis by coomassie-stained SDS-PAGE of N. benthamiana plants agroinfiltrated with construct 3431, 3432, 3433, 3434 or 5094 at an OD600 of 0.4 and expressing only the atRMD, pbRMD, psRMD, xvRMD or paRMD. As shown in FIG. 7, a band can be seen at the expected molecular weight of the atRMD (35.7 kDa), pbRMD (33.7 kDa) and paRMD (34.9 kDa) but not for psRMD (35.1 kDa) and xvRMD (33.4 kDa). However, the fact that psRMD and xvRMD could not be seen of Coomassie-stained SDS-PAGE analysis does not exclude the possibility that those RMDs are expressed in N. benthamiana but at lower level than atRMD, pbRMD, and paRMD.

Glycan Profile—Wild-Type Plants (atRMD, pbRMD, psRMD and xvRMD)

The rituximab antibody (construct 5072) was transiently expressed in wild-type Nicotiana benthamiana plants with and without the co-expression of 160/atRMD (construct 3431), 160/pbRMD (construct 3432), 160/psRMD (construct 3433) or 160/xvRMD (construct 3434) and purified as described above in example 2. N-Glycan profiling and analysis of glycopeptides of the purified antibodies was characterized using MS (LC ESI MS/MS; as described in Li et. al. (2015, Plt. Biotech. J., pp. 1-10). The N-glycosylation profile on a unique site (N301) of purified rituximab antibodies was compared to that of wild-type plants. The results are presented in Table 8, below.

TABLE 8
Comparison of N-glycan profile of the purified rituximab antibody produced in wild-type plants, with and without co-expression of atRMD,
pbRMD, psRMD, xvRMD under the control of CPMV 160 expression system. Bacterial vector comprising rituximab was infiltrated at an OD600 of
0.5 while the bacterial vector comprising RMD, when present, was infiltrated at an OD600 of 0.25. Numbers represent the average percentage
of each glycoform identified from each condition.

| | 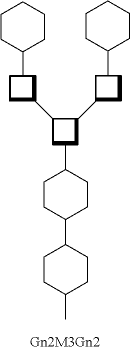 | 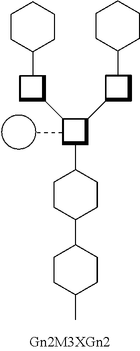 | 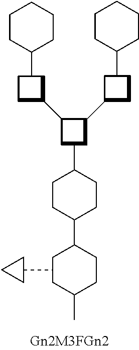 | 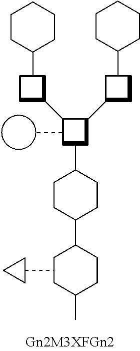 | 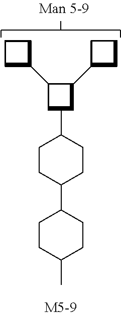 |
|---|---|---|---|---|---|
| Wild-type | 6 | 6 | 7 | 71 | 11 |
| Wild-type + paRMD | 16 | 50 | 2 | 24 | 8 |
| Wild-type + atRMD | 13 | 40 | 3 | 37 | 7 |
| Wild-type + pbRMD | 14 | 42 | 3 | 35 | 7 |
| Wild-type + psRMD | 16 | 51 | 2 | 24 | 6 |
| Wild-type + xvRMD | 14 | 36 | 4 | 40 | 7 |

Hexagon: N-acetylglucosamine; Square: mannose; Circle: xylose; Triangle: Fucose

A reduction in fucosylation was observed in plants co-expressing rituximab and atRMD, pbRMD, psRMD, xvRMD consistent with the reduction in fucosylation observed using paRMD as shown in Table 5. When produced in wild-type plants, the predominant N-glycans in the antibody had α(1,3)-fucose (the sum of percentages of glycoforms Gn2M3FGn2 and Gn2M3XFGn2). In contrast, glycosylation analysis of plants comprising atRMD, pbRMD, psRMD, or xvRMD each under the control of CPMV 160 expression system revealed a reduction of the N-glycans comprising α(1,3)-fucose, similar to that observed with paRMD (see Table 5).

### Glycan Profile—FucT/XylT Knockout Plants

The rituximab antibody (construct 5072) was also transiently expressed in wild-type or knocked-out Nicotiana benthamiana plants lines (plant line NB14-29aT2; WO 2014/071039; Li et. al. 2015, Plt. Biotech. J. p 1-10; which are incorporated herein by reference) with and without the co-expression of 160/atRMD (construct 3431), 160/pbRMD (construct 3432), 160/psRMD (construct 3433) or 160/xvRMD (construct 3434,) and purified as described above. In the Cellectis plants two (1,3)-fucosyltransferase (FucT) genes and two β(1,2)-xylosyltransferase (XylT) genes have been knocked out.

N-Glycan profiling and analysis of glycopeptides of the purified antibodies was characterized using MS (LC ESI MS/MS; as described in Li et. al. (2015, Plt. Biotech. J., pp. 1-10). The results are presented in Table 9.

TABLE 9
Comparison of N-glycan profile of the purified rituximab antibody produced in wild-type plants and FucT/XylT knockout (NB14-29aT2)
plants, with and without co-expression of 160/atRMD (construct 3431), 160/pbRMD (construct 3432), 160/psRMD (construct 3433) or 160/xvRMD
(construct 3434). The bacterial vector comprising rituximab was infiltrated at an OD600 of 0.5 while the bacterial vector comprising each of the
RMDs, when present, was infiltrated at an OD of 0.25. Numbers represent the average percentage of each glycoform identified from each plant line.

| | 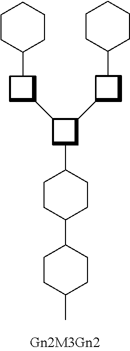 | 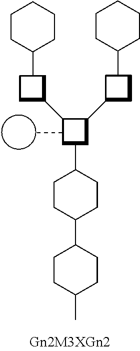 | 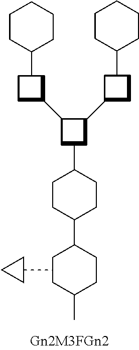 | 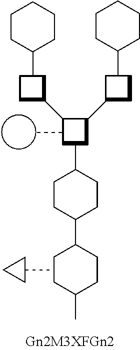 | 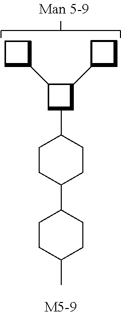 |
|---|---|---|---|---|---|
| FucT/XylT Knockout | 49 | 0 | 42 | 0 | 8 |
| FucT/XylT | 78 (82) | 0 (0) | 11 (12) | 0 (0) | 6 (3) |
| Knockout + paRMD | | | | | |
| FucT/XylT | 74 (73) | 0 (0) | 17 (20) | 0 (0) | 5 (4) |
| Knockout + atRMD | | | | | |
| FucT/XylT | 71 | 0 | 18 | 0 | 7 |
| Knockout + pbRMD | | | | | |
| FucT/XylT | 76 | 0 | 12 | 0 | 7 |
| Knockout + psRMD | | | | | |
| FucT/XylT | 78 (79) | 0 (0) | 10 (15) | 0 (0) | 7 (3) |
| Knockout + xvRMD | | | | | |

Hexagon: N-acetylglucosamine; Square: mannose; Circle: xylose; Triangle: Fucose; numbers in brackets represent data from repeated experiments

Glycosylation analysis of the FucT/XylT knockout plants (plant line NB14-29aT2; WO 2014/071039; Li et. al. 2015, Plt. Biotech. J. p 1-10; which are incorporated herein by reference) showed that 50% of the N-glycans did not have α(1,3)-fucose (the sum of percentages of glycoforms Gn2M3FGn2 and M5-9), 49% were α(1,3)-fucose-containing N-glycans (glycoform Gn2M3FGn2). When 160/atRMD was expressed in these plants 83-85% of the N-glycans did not have α(1,3)-fucose, and only 15-17%% were α(1,3)-fucose-containing N-glycans similar to the results shown in Table 6 for 160/paRMD. Similar results were achieved when atRMD, pbRMD, psRMD, xvRMD were expressed in FucT/XylT knockout plants. (see Table 9).

These results demonstrate that RMD from a variety of bacterial sources, including, but not limited to paRMD, atRMD, pbRMD, psRMD, xvRMD may be used to reduce N-glycans comprising α(1,3)-fucose in a protein of interest, when the protein of interest is co-expressed with the RMD.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A method of producing a protein of interest comprising N-glycans having a reduced fucose content in a plant, a portion of a plant, or a plant cell comprising, co-expressing within the plant, the portion of the plant, or the plant cell, a first nucleotide sequence encoding a GDP-4-dehydro-6-deoxy-D-mannose reductase (RMD), the first nucleotide sequence operatively linked with a first regulatory region that is active in the plant, the portion of a plant, or the plant cell, and a second nucleotide sequence encoding the protein of interest, the second nucleotide sequence operatively linked with a second regulatory region that is active in the plant, the portion of a plant, or the plant cell, and co-expressing the first and second nucleotide sequences to synthesize a protein of interest comprising N-glycans having a reduced fucose content, when compared to the fucose content of the protein of interest expressed in a second plant, a portion of the second plant, or a second plant cell that does not express RMD, wherein the plant, portion of the plant, or the plant cell exhibits reduced fucosylation activity resulting from both
reduced α(1,3)-fucosyltransferase (FucT) activity when compared to the FucT activity of a wildtype plant, and
from expressing the RMD.

2. (canceled)

3. The method of claim 1, wherein the plant, portion of the plant, or the plant cell additionally exhibits reduced β(1,2)-xylosyltransferase (XylT) activity.

4. The method of claim 1, wherein the protein of interest comprises reduced levels of at least one of Gn2M3FGn2 and Gn2M3XFGn2.

5. The method of claim 1, wherein at least one of FucT genes in the plant, the portion of the plant, or the plant cell is knocked out.

6. The method of claim 1, wherein the α(1,3)-fucosyltransferase (FucT) activity is further reduced using RNAi, chemical inhibition, or both.

7. The method of claim 1, wherein the first regulatory region comprises an expression enhancer.

8. The method of claim 6, wherein the expression enhancer is selected from the group consisting of CPMVX, CPMVX+, CPMV-HT+CPMV HT+[WT115] and CPMV HT+[511].

9. The method of claim 1, wherein the RMD is derived from Pseudomonas, Xanthomonas or Agrobacterium.

10. The method of claim 1, wherein the protein of interest is a therapeutic protein, an antibody, a vaccine component or a viral protein.

11. A plant, a portion of a plant, or a plant cell comprising a nucleotide sequence encoding a GDP-4-dehydro-6-deoxy-D-mannose reductase (RMD), the nucleotide sequence operatively linked with a regulatory region that is active in the plant, the portion of the plant, or the plant cell, wherein the plant, the portion of the plant, or the plant cell exhibits reduced α(1,3)-fucosyltransferase (FucT) activity when compared to the FucT activity of a wildtype plant.

12. The plant, the portion of the plant, or the plant cell of claim 10, further comprising a second nucleotide sequence for encoding a protein of interest, the second nucleotide sequence operatively linked with a second regulatory region that is active in the plant.

13. The plant, the portion of the plant, or the plant cell of claim 11, wherein the plant, the portion of the plant, or the plant cell comprise reduced β(1,2)-xylosyltransferase (XylT) activity.

14. A method for producing a protein of interest in a plant, a portion of a plant, or a plant cell, of the Nicotiana spp having at least one of its α(1,3)-fucosyltransferase (FucT) allele knocked out comprising, co-expressing a GDP-4-dehydro-6-deoxy-D-mannose reductase (RMD) and the protein of interest within the plant, the portion of the plant, or the plant cell, to produce the protein of interest having a reduced fucosylation profile when compared to the same protein of interest produced in a second plant, a portion of the second plant, or a second plant cell having at least one of its FucT allele knocked out, and that does not express RMD, wherein the plant, the portion of the plant, or the plant cell exhibits reduced α(1,3)-fucosyltransferase (FucT) activity when compared to the FucT activity of a wildtype plant.

15. (canceled)

16. A method of producing a protein of interest comprising N-glycans having a modified N-glycosylation profile in a plant, a portion of a plant, or a plant cell comprising, co-expressing within the plant, the portion of the plant, or the plant cell, a first nucleotide sequence encoding a GDP-4-dehydro-6-deoxy-D-mannose reductase (RMD) the first nucleotide sequence operatively linked with a first regulatory region that is active in the plant, the portion of the plant, or the plant cell, and a second nucleotide sequence encoding the protein of interest, the second nucleotide sequence operatively linked with a second regulatory region that is active in the plant, the portion of the plant, or the plant cell, and co-expressing the first and second nucleotide sequences to synthesize a protein of interest comprising glycans with the modified N-glycosylation profile, when compared to the N-glycosylation profile of the protein of interest expressed in a second plant, a portion of a second plant, or a second plant cell that does not express RMD, wherein the plant, portion of the plant, or the plant cell exhibits reduced α(1,3)-fucosyltransferase (FucT) activity when compared to the FucT activity of a wildtype plant.

17. The method of claim 14, wherein the N-glycans are at least one of Gn2M3FGn2 and Gn2M3XFGn2.


SEQUENCE LISTING (63 sequences)

SEQ ID NO: 1 - DNA, 160 bp, Artificial Sequence
  Expression enhancer CPMVX
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtaca

SEQ ID NO: 2 - DNA, 5 bp, Artificial Sequence
  Kozak sequence - Plant Kingdom
  misc_feature (4)..(4): A or C
ccana

SEQ ID NO: 3 - DNA, 5 bp, Artificial Sequence
  Plant Kozak sequence - dicots
  misc_feature (4)..(4): A or C
aaana

SEQ ID NO: 4 - DNA, 5 bp, Artificial Sequence
  Plant Kozak Sequence arabidopsis
  misc_feature (3)..(3): A or G
  misc_feature (4)..(4): A or C
aanna

SEQ ID NO: 5 - DNA, 5 bp, Artificial Sequence
  Plant Kozak sequence
agaaa

SEQ ID NO: 6 - DNA, 5 bp, Artificial Sequence
  Plant Kozak Sequence
agaca

SEQ ID NO: 7 - DNA, 5 bp, Artificial Sequence
  Plant Kozak Sequence
aggaa

SEQ ID NO: 8 - DNA, 5 bp, Artificial Sequence
  Plant Kozak Sequence
aaaaa

SEQ ID NO: 9 - DNA, 5 bp, Artificial Sequence
  Plant Kozak Sequence
aaaca

SEQ ID NO: 10 - DNA, 5 bp, Artificial Sequence
  Plant Kozak sequence
aagca

SEQ ID NO: 11 - DNA, 5 bp, Artificial Sequence
  Plant Kozak Sequence
aagaa

SEQ ID NO: 12 - DNA, 6 bp, Artificial Sequence
  Plant Kozak Sequence
aaagaa

SEQ ID NO: 13 - DNA, 6 bp, Artificial Sequence
  Plant Kozak Sequence
aaagaa

SEQ ID NO: 14 - DNA, 6 bp, Artificial Sequence
  Consensus Sequence
  misc_feature (1)..(1): A or absent
  misc_feature (3)..(3): A or G
  misc_feature (4)..(4): A or G
  misc_feature (5)..(5): A or C
nannna

SEQ ID NO: 15 - DNA, 517 bp, Artificial Sequence
  CPMV HT expression enhancer
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggccc

SEQ ID NO: 16 - DNA, 533 bp, Artificial Sequence
  CPMV HT+ with a plant kozak consensus sequence
  misc_feature (529)..(529): A or absent
  misc_feature (531)..(532): A or G
  misc_feature (533)..(533): A or C
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgttcgggcccaataccgcggnanna

SEQ ID NO: 17 - DNA, 511 bp, Artificial Sequence
  CPMV HT+ 511
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgaa

SEQ ID NO: 18 - DNA, 534 bp, Artificial Sequence
  CPMV HT+[WT115]
tattaaaatcttaataggttttgataaaagcgaacgtggggaaacccgaaccaaaccttcttctaaactctctctcatctctcttaaagcaaacttctctcttgtctttcttgcatgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgttcgggcccaataccgcggagaaaa

SEQ ID NO: 19 - DNA, 44 bp, Artificial Sequence
  Primer Flag_Rmd_Fw
gactacaaagacgatgacgacaagacgcagcgcctgtttgtcac

SEQ ID NO: 20 - DNA, 38 bp, Artificial Sequence
  Primer 5091_5092_IF_Rev
actaaagaaaataggccttcattcctccctcacgcggc

SEQ ID NO: 21 - DNA, 915 bp, Pseudomonas aeruginosa
atgacgcagcgcctgtttgtcaccggacttagtgggttcgtgggaaagcacctacaggcatatcttgccgccgcacataccccctgggcgctgctgcctgtgccacaccggtacgatctactcgagccagactccttgggagacctttggcctgagttacccgatgcagtcatccatcttgccggccagacatatgtaccagaggctttccgtgatccagcccgcaccttgcagatcaatctgctgggcactctgaacttgctccaagctttaaaagctcggggctttagcgggacttttctgtatataagctcgggggatgtgtacggccaggtcgccgaggccgcccttccaatacacgaagagctgatacctcaccccagaaatccatacgcagtgtccaaactggccgctgaaagcctttgtctgcagtggggcatcacagagggatggcgggtgctcgtagctaggccattcaatcacatcggtcccggccagaaggatagctttgttattgcctccgctgccagacaaatcgcgaggatgaagcaaggcttgcaggccaatcggttggaagtcggagacatcgacgtgtcgagagacttcctggatgtccaggatgtgctgtccgcctacctaagattactgtctcatggcgaagcgggcgcagtttataatgtgtgcagcggccaggagcagaagattagagagctgatcgagttgctggccgatattgcccaggtggagcttgaaattgttcaggacccggcccgaatgagaagggctgagcaaagacgtgtgaggggcagccacgcccgtctgcacgatacaactggttggaagccagagattacaataaaacagtctcttagagcaattctctcagactgggagagccgcgtgagggaggaatga

SEQ ID NO: 22 - DNA, 47 bp, Artificial Sequence
  Primer 5091_IF_Fw
acagggcccaataccgcggagaaaatggactacaaagacgatgacga

SEQ ID NO: 23 - DNA, 4644 bp, Artificial Sequence
  Construct 2171
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacagggcccaataccgcggagaaaatggcgaaaaacgttgcgattttcggcttattgttttctcttcttgtgttggttccttctcagatcttcgcgacgtcactcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagacgtccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 24 - DNA, 2343 bp, Artificial Sequence
  Expression cassetts 5091
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacagggcccaataccgcggagaaaatggactacaaagacgatgacgacaagacgcagcgcctgtttgtcaccggacttagtgggttcgtgggaaagcacctacaggcatatcttgccgccgcacataccccctgggcgctgctgcctgtgccacaccggtacgatctactcgagccagactccttgggagacctttggcctgagttacccgatgcagtcatccatcttgccggccagacatatgtaccagaggctttccgtgatccagcccgcaccttgcagatcaatctgctgggcactctgaacttgctccaagctttaaaagctcggggctttagcgggacttttctgtatataagctcgggggatgtgtacggccaggtcgccgaggccgcccttccaatacacgaagagctgatacctcaccccagaaatccatacgcagtgtccaaactggccgctgaaagcctttgtctgcagtggggcatcacagagggatggcgggtgctcgtagctaggccattcaatcacatcggtcccggccagaaggatagctttgttattgcctccgctgccagacaaatcgcgaggatgaagcaaggcttgcaggccaatcggttggaagtcggagacatcgacgtgtcgagagacttcctggatgtccaggatgtgctgtccgcctacctaagattactgtctcatggcgaagcgggcgcagtttataatgtgtgcagcggccaggagcagaagattagagagctgatcgagttgctggccgatattgcccaggtggagcttgaaattgttcaggacccggcccgaatgagaagggctgagcaaagacgtgtgaggggcagccacgcccgtctgcacgatacaactggttggaagccagagattacaataaaacagtctcttagagcaattctctcagactgggagagccgcgtgagggaggaatgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 25 - protein, 312 aa, Pseudomonas aeruginosa
MDYKDDDDKTQRLFVTGLSGFVGKHLQAYLAAAHTPWALLPVPHRYDLLEPDSLGDLWPELPDAVIHLAGQTYVPEAFRDPARTLQINLLGTLNLLQALKARGFSGTFLYISSGDVYGQVAEAALPIHEELIPHPRNPYAVSKLAAESLCLQWGITEGWRVLVARPFNHIGPGQKDSFVIASAARQIARMKQGLQANRLEVGDIDVSRDFLDVQDVLSAYLRLLSHGEAGAVYNVCSGQEQKIRELIELLADIAQVELEIVQDPARMRRAEQRRVRGSHARLHDTTGWKPEITIKQSLRAILSDWESRVREE

SEQ ID NO: 26 - DNA, 44 bp, Artificial Sequence
  Primer 5092_IF_Fw
tcgtgcttcggcaccagtacaatggactacaaagacgatgacga

SEQ ID NO: 27 - DNA, 4540 bp, Artificial Sequence
  Construct 1190
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 28 - DNA, 2322 bp, Artificial Sequence
  Expression cassette 5092
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggactacaaagacgatgacgacaagacgcagcgcctgtttgtcaccggacttagtgggttcgtgggaaagcacctacaggcatatcttgccgccgcacataccccctgggcgctgctgcctgtgccacaccggtacgatctactcgagccagactccttgggagacctttggcctgagttacccgatgcagtcatccatcttgccggccagacatatgtaccagaggctttccgtgatccagcccgcaccttgcagatcaatctgctgggcactctgaacttgctccaagctttaaaagctcggggctttagcgggacttttctgtatataagctcgggggatgtgtacggccaggtcgccgaggccgcccttccaatacacgaagagctgatacctcaccccagaaatccatacgcagtgtccaaactggccgctgaaagcctttgtctgcagtggggcatcacagagggatggcgggtgctcgtagctaggccattcaatcacatcggtcccggccagaaggatagctttgttattgcctccgctgccagacaaatcgcgaggatgaagcaaggcttgcaggccaatcggttggaagtcggagacatcgacgtgtcgagagacttcctggatgtccaggatgtgctgtccgcctacctaagattactgtctcatggcgaagcgggcgcagtttataatgtgtgcagcggccaggagcagaagattagagagctgatcgagttgctggccgatattgcccaggtggagcttgaaattgttcaggacccggcccgaatgagaagggctgagcaaagacgtgtgaggggcagccacgcccgtctgcacgatacaactggttggaagccagagattacaataaaacagtctcttagagcaattctctcagactgggagagccgcgtgagggaggaatgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 29 - DNA, 47 bp, Artificial Sequence
  Primer 5093_IF_Fw
acagggcccaataccgcggagaaaatgacgcagcgcctgtttgtcac

SEQ ID NO: 30 - DNA, 2319 bp, Artificial Sequence
  Expression cassette 5093
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacagggcccaataccgcggagaaaatgacgcagcgcctgtttgtcaccggacttagtgggttcgtgggaaagcacctacaggcatatcttgccgccgcacataccccctgggcgctgctgcctgtgccacaccggtacgatctactcgagccagactccttgggagacctttggcctgagttacccgatgcagtcatccatcttgccggccagacatatgtaccagaggctttccgtgatccagcccgcaccttgcagatcaatctgctgggcactctgaacttgctccaagctttaaaagctcggggctttagcgggacttttctgtatataagctcgggggatgtgtacggccaggtcgccgaggccgcccttccaatacacgaagagctgatacctcaccccagaaatccatacgcagtgtccaaactggccgctgaaagcctttgtctgcagtggggcatcacagagggatggcgggtgctcgtagctaggccattcaatcacatcggtcccggccagaaggatagctttgttattgcctccgctgccagacaaatcgcgaggatgaagcaaggcttgcaggccaatcggttggaagtcggagacatcgacgtgtcgagagacttcctggatgtccaggatgtgctgtccgcctacctaagattactgtctcatggcgaagcgggcgcagtttataatgtgtgcagcggccaggagcagaagattagagagctgatcgagttgctggccgatattgcccaggtggagcttgaaattgttcaggacccggcccgaatgagaagggctgagcaaagacgtgtgaggggcagccacgcccgtctgcacgatacaactggttggaagccagagattacaataaaacagtctcttagagcaattctctcagactgggagagccgcgtgagggaggaatgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 31 - protein, 304 aa, Pseudomonas aeruginosa
MTQRLFVTGLSGFVGKHLQAYLAAAHTPWALLPVPHRYDLLEPDSLGDLWPELPDAVIHLAGQTYVPEAFRDPARTLQINLLGTLNLLQALKARGFSGTFLYISSGDVYGQVAEAALPIHEELIPHPRNPYAVSKLAAESLCLQWGITEGWRVLVARPFNHIGPGQKDSFVIASAARQIARMKQGLQANRLEVGDIDVSRDFLDVQDVLSAYLRLLSHGEAGAVYNVCSGQEQKIRELIELLADIAQVELEIVQDPARMRRAEQRRVRGSHARLHDTTGWKPEITIKQSLRAILSDWESRVREE

SEQ ID NO: 32 - DNA, 44 bp, Artificial Sequence
  Primer 5094_IF_Fw
tcgtgcttcggcaccagtacaatgacgcagcgcctgtttgtcac

SEQ ID NO: 33 - DNA, 2298 bp, Artificial Sequence
  Expression cassette 5094
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatgacgcagcgcctgtttgtcaccggacttagtgggttcgtgggaaagcacctacaggcatatcttgccgccgcacataccccctgggcgctgctgcctgtgccacaccggtacgatctactcgagccagactccttgggagacctttggcctgagttacccgatgcagtcatccatcttgccggccagacatatgtaccagaggctttccgtgatccagcccgcaccttgcagatcaatctgctgggcactctgaacttgctccaagctttaaaagctcggggctttagcgggacttttctgtatataagctcgggggatgtgtacggccaggtcgccgaggccgcccttccaatacacgaagagctgatacctcaccccagaaatccatacgcagtgtccaaactggccgctgaaagcctttgtctgcagtggggcatcacagagggatggcgggtgctcgtagctaggccattcaatcacatcggtcccggccagaaggatagctttgttattgcctccgctgccagacaaatcgcgaggatgaagcaaggcttgcaggccaatcggttggaagtcggagacatcgacgtgtcgagagacttcctggatgtccaggatgtgctgtccgcctacctaagattactgtctcatggcgaagcgggcgcagtttataatgtgtgcagcggccaggagcagaagattagagagctgatcgagttgctggccgatattgcccaggtggagcttgaaattgttcaggacccggcccgaatgagaagggctgagcaaagacgtgtgaggggcagccacgcccgtctgcacgatacaactggttggaagccagagattacaataaaacagtctcttagagcaattctctcagactgggagagccgcgtgagggaggaatgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 34 - DNA, 52 bp, Artificial Sequence
  Primer IF**(SacII)-PDI.s1+4c
acagggcccaataccgcggagaaaatggcgaaaaacgttgcgattttcggct

SEQ ID NO: 35 - DNA, 51 bp, Artificial Sequence
  Primer IF**-HC(Ritux).s1-6r
actaaagaaaataggccttcactttccaggagaaagagaaagggacttttg

SEQ ID NO: 36 - DNA, 1428 bp, Artificial Sequence
  Coding sequence of PDISP/HC Rituxan
atggcgaaaaacgttgcgattttcggcttattgttttctcttcttgtgttggttccttctcagatcttcgcgcaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctagggaaccacaagtgtacactcttccaccatctagggatgagcttactaagaaccaagtttctcttacttgtcttgtgaagggattttatccatctgacatcgccgtggaatgggaatccaacggacaaccagagaacaattacaagactactccaccagttcttgattctgatggatccttctttctttattccaagcttactgttgataagtccagatggcagcaaggaaatgtgttctcttgttctgttatgcacgaagctcttcataatcattatactcaaaagtccctttctctttctcctggaaagtga

SEQ ID NO: 37 - DNA, 2832 bp, Artificial Sequence
  Expression cassette 2109
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacagggcccaataccgcggagaaaatggcgaaaaacgttgcgattttcggcttattgttttctcttcttgtgttggttccttctcagatcttcgcgcaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctagggaaccacaagtgtacactcttccaccatctagggatgagcttactaagaaccaagtttctcttacttgtcttgtgaagggattttatccatctgacatcgccgtggaatgggaatccaacggacaaccagagaacaattacaagactactccaccagttcttgattctgatggatccttctttctttattccaagcttactgttgataagtccagatggcagcaaggaaatgtgttctcttgttctgttatgcacgaagctcttcataatcattatactcaaaagtccctttctctttctcctggaaagtgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 38 - protein, 475 aa, Artificial Sequence
  PDISP/HC Rituxan monoclonal antibody
MAKNVAIFGLLFSLLVLVPSQIFAQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 51 bp, Artificial Sequence
  Primer IF**-LC(Ritux).s1-6r
actaaagaaaataggccttcaacactctcccctgttgaagctctttgtgac

SEQ ID NO: 40 - DNA, 714 bp, Artificial Sequence
  Coding sequence of PDISP/HC Rituxan
atggcgaaaaacgttgcgattttcggcttattgttttctcttcttgtgttggttccttctcagatcttcgcgcaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcacaatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 41 - DNA, 2118 bp, Artificial Sequence
  Expression cassette 2129
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacagggcccaataccgcggagaaaatggcgaaaaacgttgcgattttcggcttattgttttctcttcttgtgttggttccttctcagatcttcgcgcaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcacaatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 42 - protein, 237 aa, Artificial Sequence
  PDISP/LC Rituxan monoclonal antibody
MAKNVAIFGLLFSLLVLVPSQIFAQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 5073 bp, Artificial Sequence
  Expression cassette 5072
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacagggcccaataccgcggagaaaatggcgaaaaacgttgcgattttcggcttattgttttctcttcttgtgttggttccttctcagatcttcgcgcaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctagggaaccacaagtgtacactcttccaccatctagggatgagcttactaagaaccaagtttctcttacttgtcttgtgaagggattttatccatctgacatcgccgtggaatgggaatccaacggacaaccagagaacaattacaagactactccaccagttcttgattctgatggatccttctttctttattccaagcttactgttgataagtccagatggcagcaaggaaatgtgttctcttgttctgttatgcacgaagctcttcataatcattatactcaaaagtccctttctctttctcctggaaagtgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacagggcccaataccgcggagaaaatggcgaaaaacgttgcgattttcggcttattgttttctcttcttgtgttggttccttctcagatcttcgcgcaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcacaatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 44 - DNA, 48 bp, Artificial Sequence
  IF-atRMD(opt).c
tcgtgcttcggcaccagtacaatggcccagcagaacagcgtttccaac

SEQ ID NO: 45 - DNA, 48 bp, Artificial Sequence
  IF-atRMD(opt).r
actaaagaaaataggccttcagtcggatgcgcgccaataatccagcac

SEQ ID NO: 46 - DNA, 996 bp, Agrobacterium tumefaciens
atggcccagcagaacagcgtttccaacccccggatcctcatcactggggccaatgggttcgtcggccgatggctccgggaggagctccaaaaggtacttcctccggcagcgcaaatcatctccaccttcaaaggtggagggaacgacgtaaccgctcagtcccaagggacggaggtgagcatcgcactcgacattgaagacgaagaggccgtggcacatgtaatcgaaacttggagacccaccgtggtgatacatctggccgcagtgtcacatgtgcttgaagcatcggcctcacctaaacaaactttcgcagtaaacttgaacggtactattgctctggcctgttcagtgaggaaattctgccctgacgctgtgttcattaacattggatcatccgaagtgtatggaagcagcttcatcggggctacgaggcctctggatgaggaaacccttctggctcctactaacgtgtacgccacatctaaggctgccgctgatctgtttattgggcagatgtcaaagaatggcctcaacgctgtaaggttcaggccattcaatcacacgggccctggccaggcagagaacttcgtggtgtctggctttgctgctcaaattgccagaattgagaagggattgcagccacccattatcaaggtcgggaatctggaggcacagagggatttcacagacgttcgcgatattgtgtcggcatacgtcgcagccgcccttctgccccccgagcgtttcagggacggggtcatacttaacctgagtagcggaaccgcgaggaggatcggggacatcttagactctctgcttgctatgtcccaggttaaaatccaggtactggaggaccctgcacgcatgagagcctcagaggtgagctatgtggcaggaagttcagccaaagccgggcacttgctccagtggagcccccagatcccctgggaaacaaccctgcggtccgtgctggattattggcgcgcatccgactga

SEQ ID NO: 47 - DNA, 2379 bp, Artificial Sequence
  Expression cassette number 3431
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcccagcagaacagcgtttccaacccccggatcctcatcactggggccaatgggttcgtcggccgatggctccgggaggagctccaaaaggtacttcctccggcagcgcaaatcatctccaccttcaaaggtggagggaacgacgtaaccgctcagtcccaagggacggaggtgagcatcgcactcgacattgaagacgaagaggccgtggcacatgtaatcgaaacttggagacccaccgtggtgatacatctggccgcagtgtcacatgtgcttgaagcatcggcctcacctaaacaaactttcgcagtaaacttgaacggtactattgctctggcctgttcagtgaggaaattctgccctgacgctgtgttcattaacattggatcatccgaagtgtatggaagcagcttcatcggggctacgaggcctctggatgaggaaacccttctggctcctactaacgtgtacgccacatctaaggctgccgctgatctgtttattgggcagatgtcaaagaatggcctcaacgctgtaaggttcaggccattcaatcacacgggccctggccaggcagagaacttcgtggtgtctggctttgctgctcaaattgccagaattgagaagggattgcagccacccattatcaaggtcgggaatctggaggcacagagggatttcacagacgttcgcgatattgtgtcggcatacgtcgcagccgcccttctgccccccgagcgtttcagggacggggtcatacttaacctgagtagcggaaccgcgaggaggatcggggacatcttagactctctgcttgctatgtcccaggttaaaatccaggtactggaggaccctgcacgcatgagagcctcagaggtgagctatgtggcaggaagttcagccaaagccgggcacttgctccagtggagcccccagatcccctgggaaacaaccctgcggtccgtgctggattattggcgcgcatccgactgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 48 - protein, 331 aa, Agrobacterium tumefaciens
MAQQNSVSNPRILITGANGFVGRWLREELQKVLPPAAQIISTFKGGGNDVTAQSQGTEVSIALDIEDEEAVAHVIETWRPTVVIHLAAVSHVLEASASPKQTFAVNLNGTIALACSVRKFCPDAVFINIGSSEVYGSSFIGATRPLDEETLLAPTNVYATSKAAADLFIGQMSKNGLNAVRFRPFNHTGPGQAENFVVSGFAAQIARIEKGLQPPIIKVGNLEAQRDFTDVRDIVSAYVAAALLPPERFRDGVILNLSSGTARRIGDILDSLLAMSQVKIQVLEDPARMRASEVSYVAGSSAKAGHLLQWSPQIPWETTLRSVLDYWRASD

SEQ ID NO: 49 - DNA, 48 bp, Artificial Sequence
  IF-pbRMD(opt).c
tcgtgcttcggcaccagtacaatgaagaagagactgttcgtaactggt

SEQ ID NO: 50 - DNA, 52 bp, Artificial Sequence
  IF-pbRMD(opt).r
actaaagaaaataggccttcactcttgctgcacccttatctcccaatcgcta

SEQ ID NO: 51 - DNA, 915 bp, pseudomonas brassicacearum
atgaagaagagactgttcgtaactggtttgtccggatttgtgggaaggcatctgaagtctcgcctgaacggacacgacctcgcctggcaagtgatgccagtagccgcccgttatgatctgatggatgcaaaaacactggaagggctgtggcccgagatcccagatgcggttatccacttggccggtcagaccttcgtccccgaggccttcagggaccccgcacgcacactacatatcaacctcctgggcacactgaatctgctgcaggcactcaagacaagaggcttcactggcacatttctgtatgtgtctagcggggatgtgtacggtcaggtctccgaagaccatctgcctatcaccgaattgcagttgcccagtccaaggaatccctacgctgtgagcaagctctccgcagaacttctcagtctgcagtggggaatgtccgagggttggcccgtgttagttgctcgcccattcaatcacatcggtccaggccagaaagattcgttcgtgatagcatcggcagctcgtcaaatcagccggatacgcctgggcctgcagcctgcaagactgcaggttggggacattgatgtgacccgcgacttcctggatgtcggtgacgtgatttcggcttacgtggcccttttggacaagggagcacccggtcaggtttataatatatgttctggacgcgaacagtcgattaggtctctgatcgagcaacttggcgacatcgctcaaatcgacgtgcaactgatccaggatcctgctcggctgaggagagctgaacagagaagagtttgtgggtccccagctaagctgaggcaggcgactggctggaccccagagactacaacacaacagagtctcagggccatacttagcgattgggagataagggtgcagcaagagtga

SEQ ID NO: 52 - DNA, 2298 bp, Artificial Sequence
  Expression cassette number 3432
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatgaagaagagactgttcgtaactggtttgtccggatttgtgggaaggcatctgaagtctcgcctgaacggacacgacctcgcctggcaagtgatgccagtagccgcccgttatgatctgatggatgcaaaaacactggaagggctgtggcccgagatcccagatgcggttatccacttggccggtcagaccttcgtccccgaggccttcagggaccccgcacgcacactacatatcaacctcctgggcacactgaatctgctgcaggcactcaagacaagaggcttcactggcacatttctgtatgtgtctagcggggatgtgtacggtcaggtctccgaagaccatctgcctatcaccgaattgcagttgcccagtccaaggaatccctacgctgtgagcaagctctccgcagaacttctcagtctgcagtggggaatgtccgagggttggcccgtgttagttgctcgcccattcaatcacatcggtccaggccagaaagattcgttcgtgatagcatcggcagctcgtcaaatcagccggatacgcctgggcctgcagcctgcaagactgcaggttggggacattgatgtgacccgcgacttcctggatgtcggtgacgtgatttcggcttacgtggcccttttggacaagggagcacccggtcaggtttataatatatgttctggacgcgaacagtcgattaggtctctgatcgagcaacttggcgacatcgctcaaatcgacgtgcaactgatccaggatcctgctcggctgaggagagctgaacagagaagagtttgtgggtccccagctaagctgaggcaggcgactggctggaccccagagactacaacacaacagagtctcagggccatacttagcgattgggagataagggtgcagcaagagtgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 53 - protein, 304 aa, Pseudomonas brassicacearum
MKKRLFVTGLSGFVGRHLKSRLNGHDLAWQVMPVAARYDLMDAKTLEGLWPEIPDAVIHLAGQTFVPEAFRDPARTLHINLLGTLNLLQALKTRGFTGTFLYVSSGDVYGQVSEDHLPITELQLPSPRNPYAVSKLSAELLSLQWGMSEGWPVLVARPFNHIGPGQKDSFVIASAARQISRIRLGLQPARLQVGDIDVTRDFLDVGDVISAYVALLDKGAPGQVYNICSGREQSIRSLIEQLGDIAQIDVQLIQDPARLRRAEQRRVCGSPAKLRQATGWTPETTTQQSLRAILSDWEIRVQQE

SEQ ID NO: 54 - DNA, 51 bp, Artificial Sequence
  IF-psRMD(opt).c
tcgtgcttcggcaccagtacaatgagtcggatcctcctgaccggtgctagt

SEQ ID NO: 55 - DNA, 57 bp, Artificial Sequence
  IF-psRMD(opt).r
actaaagaaaataggccttcacttggatggcgaggtttgttcccgccaataattaag

SEQ ID NO: 56 - DNA, 957 bp, Pseudomonas syringae
atgagtcggatcctcctgaccggtgctagtggattcgtgggcaaggcattacataagaggctattggaagagaaccacgaggtttttgggaccgttagaagtagctccgattccctgatgcccgatcagcaatatagtttactggacatttgcaacagggacgaggtagatgaggtggtgcagagggtaaggcccacacatctggtgcatctggcagccatttcctctgtggcaaattcgttcaaggatcctcttctaacgtggaataccaatatcattggtaccttgaatcttatggagagtctgaaaaaaaatgcgccgggatgcttcgtgctgttcgtgtctagctctgaagtctacggggaatccttcaagaccggcgaactgctgaccgaggacgccgtatgtcaccctatgaatccctacgcagcctccaagctagcggccgaaatagccttcaagcagtacctgaggcagggccaacggggcataatcgtcagacccttcaaccatatcggacccggacaatcagtcgatttcgttaccgcctctttcgcccggcagattgcactgatagaggccgggctgcagaggcctgtgttgagagtaggtaacctggaggcatcacgcgacttcctggacgtcaacgacgtgtgtgacgcttatgtgaagatactgggccaagatcagtccacatttgcgcatactgtttacaatatcagctcagggtcaacacgtaaaatacagaccgtgttgaacgagttgctggcccagaccctgcatcccatcgagatacagacagatccagaaagactgagaccctctgacatcccagtcgccgcggggtccaacgcacgtatccacgacgagctgggctggtcccccgccaccttgttttctcagacccttgcttctgtgcttaattattggcgggaacaaacctcgccatccaagtga

SEQ ID NO: 57 - DNA, 2340 bp, Artificial Sequence
  Expression cassette number 3433
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatgagtcggatcctcctgaccggtgctagtggattcgtgggcaaggcattacataagaggctattggaagagaaccacgaggtttttgggaccgttagaagtagctccgattccctgatgcccgatcagcaatatagtttactggacatttgcaacagggacgaggtagatgaggtggtgcagagggtaaggcccacacatctggtgcatctggcagccatttcctctgtggcaaattcgttcaaggatcctcttctaacgtggaataccaatatcattggtaccttgaatcttatggagagtctgaaaaaaaatgcgccgggatgcttcgtgctgttcgtgtctagctctgaagtctacggggaatccttcaagaccggcgaactgctgaccgaggacgccgtatgtcaccctatgaatccctacgcagcctccaagctagcggccgaaatagccttcaagcagtacctgaggcagggccaacggggcataatcgtcagacccttcaaccatatcggacccggacaatcagtcgatttcgttaccgcctctttcgcccggcagattgcactgatagaggccgggctgcagaggcctgtgttgagagtaggtaacctggaggcatcacgcgacttcctggacgtcaacgacgtgtgtgacgcttatgtgaagatactgggccaagatcagtccacatttgcgcatactgtttacaatatcagctcagggtcaacacgtaaaatacagaccgtgttgaacgagttgctggcccagaccctgcatcccatcgagatacagacagatccagaaagactgagaccctctgacatcccagtcgccgcggggtccaacgcacgtatccacgacgagctgggctggtcccccgccaccttgttttctcagacccttgcttctgtgcttaattattggcgggaacaaacctcgccatccaagtgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 58 - protein, 318 aa, PSEUDOMONAS SYRINGAE
MSRILLTGASGFVGKALHKRLLEENHEVFGTVRSSSDSLMPDQQYSLLDICNRDEVDEVVQRVRPTHLVHLAAISSVANSFKDPLLTWNTNIIGTLNLMESLKKNAPGCFVLFVSSSEVYGESFKTGELLTEDAVCHPMNPYAASKLAAEIAFKQYLRQGQRGIIVRPFNHIGPGQSVDFVTASFARQIALIEAGLQRPVLRVGNLEASRDFLDVNDVCDAYVKILGQDQSTFAHTVYNISSGSTRKIQTVLNELLAQTLHPIEIQTDPERLRPSDIPVAAGSNARIHDELGWSPATLFSQTLASVLNYWREQTSPSK

SEQ ID NO: 59 - DNA, 48 bp, Artificial Sequence
  IF-xvRMD(opt).c
tcgtgcttcggcaccagtacaatgaatgatgtgtgtaagaagctactc

SEQ ID NO: 60 - DNA, 49 bp, Artificial Sequence
  IF-xvRMD(opt).r
actaaagaaaataggcctttactgaatcttccgggcatgctccaggatc

SEQ ID NO: 61 - DNA, 921 bp, Xanthomonas vasicola
atgaatgatgtgtgtaagaagctactcgtgacaggggctggaggcttcgtcggtaagcatctactggacgctgtgggtcgaggtcagtttggaaatttcgaagcccttcctctcccagccggcactgatttgcgggaccgggctgccattgagtcagctttgggcgatgtctgtcctgatgctgtagtgcacctggccgcacaatcatttgttccacagagcttcgatgaccctgaagaaactctgcaaatcaatctgatcggaacgctgcacttgcttcaagccctcgcaagaaaaggcttcagtggacggtttctgtacgtgtcctccggagatatatacggacgtgttccagagtccgacctgccagtggatgagacgttactgcctgagccccgctctccttacgctgtaagtaagtgcgcagctgaacagctgtgtatgcaatggcatcgaagcgagaaactggacgtcgttatcgctcgccccttcaaccacgttggcgcaggacagggggatcggttcgtcctcagctccttagcgcggcaggtcgtcgccatcgctgaagggaggcaggctgcggtaatagaagcgggggatatcgacactacaagagacttcagtgatgttcgggacatcgtcagcgcctacgccgctctgcttaccagagggagatccggagagacatatatcgtggctagtggagtcgaacgtcgagtccgcgatctcctgatagagatgtgtagactgacgggggtcgaagctgaagtgcgacaggactctgccaagatgcgccctgctgaacaaaggcgcatggtggcttctcccgcaaaactgcaaagagacaccggctggatgcaagcattcgatatacagtccactctctccgagatcctggagcatgcccggaagattcagtaa

SEQ ID NO: 62 - DNA, 2304 bp, Artificial  Sequence
  Expression cassette number 3434
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatgaatgatgtgtgtaagaagctactcgtgacaggggctggaggcttcgtcggtaagcatctactggacgctgtgggtcgaggtcagtttggaaatttcgaagcccttcctctcccagccggcactgatttgcgggaccgggctgccattgagtcagctttgggcgatgtctgtcctgatgctgtagtgcacctggccgcacaatcatttgttccacagagcttcgatgaccctgaagaaactctgcaaatcaatctgatcggaacgctgcacttgcttcaagccctcgcaagaaaaggcttcagtggacggtttctgtacgtgtcctccggagatatatacggacgtgttccagagtccgacctgccagtggatgagacgttactgcctgagccccgctctccttacgctgtaagtaagtgcgcagctgaacagctgtgtatgcaatggcatcgaagcgagaaactggacgtcgttatcgctcgccccttcaaccacgttggcgcaggacagggggatcggttcgtcctcagctccttagcgcggcaggtcgtcgccatcgctgaagggaggcaggctgcggtaatagaagcgggggatatcgacactacaagagacttcagtgatgttcgggacatcgtcagcgcctacgccgctctgcttaccagagggagatccggagagacatatatcgtggctagtggagtcgaacgtcgagtccgcgatctcctgatagagatgtgtagactgacgggggtcgaagctgaagtgcgacaggactctgccaagatgcgccctgctgaacaaaggcgcatggtggcttctcccgcaaaactgcaaagagacaccggctggatgcaagcattcgatatacagtccactctctccgagatcctggagcatgcccggaagattcagtaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 63 - protein, 306 aa, Xanthomonas vasicola
MNDVCKKLLVTGAGGFVGKHLLDAVGRGQFGNFEALPLPAGTDLRDRAAIESALGDVCPDAVVHLAAQSFVPQSFDDPEETLQINLIGTLHLLQALARKGFSGRFLYVSSGDIYGRVPESDLPVDETLLPEPRSPYAVSKCAAEQLCMQWHRSEKLDVVIARPFNHVGAGQGDRFVLSSLARQVVAIAEGRQAAVIEAGDIDTTRDFSDVRDIVSAYAALLTRGRSGETYIVASGVERRVRDLLIEMCRLTGVEAEVRQDSAKMRPAEQRRMVASPAKLQRDTGWMQAFDIQSTLSEILEHARKIQ